(19) United States
(12) Patent Application Publication
(10) Pub. No.: US 2026/0169011 A1
(43) Pub. Date: Jun. 18, 2026

(54) METHODS AND COMPOSITIONS FOR PROVIDING CUSTOMIZED NUTRITIONAL SUPPLEMENTS

(71) Applicant: Vibrant Holdings LLC, Santa Clara, CA (US)

(72) Inventors: Hari Krishnan Krishnamurthy, San Jose, CA (US); Vasanth Jayaraman, Sunnyvale, CA (US); Kang Bei, San Mateo, CA (US); Tianhao Wang, Fremont, CA (US); John J. Rajasekaran, Hillsborough, CA (US); Karthik Krishna, San Ramon, CA (US)

(21) Appl. No.: 19/532,908

(22) Filed: Feb. 6, 2026

Related U.S. Application Data
(63) Continuation of application No. PCT/US2024/041567, filed on Aug. 8, 2024.
(60) Provisional application No. 63/531,528, filed on Aug. 8, 2023.

(51) Int. Cl. G01N 33/82; A61K 31/047; A61K 31/07; A61K 31/122; A61K 31/197; A61K 31/198; A61K 31/201; A61K 31/202; A61K 31/205; A61K 31/355; A61K 31/375; A61K 31/4415; A61K 31/455; A61K 31/51; A61K 31/519; A61K 31/525; A61K 31/593; A61K 31/714; A61K 33/00; A61K 33/04; A61K 33/06; A61K 33/18; A61K 33/24; A61K 33/26; A61K 33/30; A61K 33/32; A61K 33/34; C12Q 1/6883; G01N 33/68; G01N 33/84; G01N 33/92

(52) U.S. Cl. CPC G01N 33/82; A61K 31/047; A61K 31/07; A61K 31/122; A61K 31/197; A61K 31/198; A61K 31/201; A61K 31/202; A61K 31/205; A61K 31/355; A61K 31/375; A61K 31/4415; A61K 31/455; A61K 31/51; A61K 31/519; A61K 31/525; A61K 31/593; A61K 31/714; A61K 33/00; A61K 33/04; A61K 33/06; A61K 33/18; A61K 33/24; A61K 33/26; A61K 33/30; A61K 33/32; A61K 33/34; C12Q 1/6883; G01N 33/68; G01N 33/84; G01N 33/92; C12Q 2600/106; C12Q 2600/118; C12Q 2600/156; G01N 2800/52

(57) ABSTRACT

Provided herein are methods for providing personalized nutritional supplementation to a subject. Also provided are compositions of personalized nutritional supplementation for subjects in need thereof.

### CROSS REFERENCE TO RELATED APPLICATIONS

This application claims the benefit of and priority to the International Patent Application under the Patent Cooperation Treaty with International Application No. PCT/US2024/041567, filed on Aug. 8, 2024, which claims the benefit of and priority to the U.S. Provisional Patent Application No. 63/531,528, filed on Aug. 8, 2023, the entire content of each of which is incorporated herein by reference in their entireties.

### BACKGROUND

Nutrients such as vitamins, minerals, amino acids, and fatty acids are required in small amounts and they play a role in virtually all metabolic processes. They act as coenzymes and cofactors in various biological processes. They are also known to aid in functions like maintenance of tissue function, activation of genes, and regulation of gene transcription. These nutrients are also important in bringing about antioxidant and inflammatory processes. Given their vital roles in numerous biological functions, even a marginal lack of a particular nutrient could prove detrimental to the body. For example, a vitamin A deficiency can give rise to night blindness and xerophthalmia. Deficiency in the B complex vitamins leads to several clinical conditions ranging from megaloblastic anemia, pellagra, beriberi to even neurological and cognitive impairments. Scurvy, a condition wherein the patient experiences weakness and has sore arms and legs is caused by the lack of vitamin C. Calcium deficiency could result in decreased bone mineralization, rickets, or osteoporosis. Selenium deficiency exposes the individual to the risk of cardiomyopathy and cardiovascular conditions. Increased dental decay and affected bone health could indicate a fluoride deficiency. While iodine deficiency could give rise to conditions like goiter and hypothyroidism, an iron deficiency can give rise to anemia.

Nutrient levels can be optimized via supplementation with nutrient-rich foods and supplements. However, nutrient supplementation can be toxic when taken excessively. While readily available ‘over-the-counter’ vitamins and minerals have been extensively used and have proven to improve health, studies have documented the rise of serious health risks associated with the same. Excess supplementation of vitamin B6 (doses higher than 500 mg/d) was associated with chronic sensory polyneuropathy in elderly patients consuming multivitamin supplements. Similarly, health conditions and lifestyle habits are important aspects that need to be factored in while supplementing nutrients. Excess intake of vitamin A (retinol) during pregnancy may increase the risk of birth defects in infants. Moreover, smokers who consumed beta-carotene supplements were associated with a higher incidence of lung cancer during 5-8 years of follow-up than smokers who did not consume beta-carotene supplements. As a result, various factors need to be taken into consideration during nutrient supplementation.

Thus, for efficacious supplementation of nutrients, an effective diagnosis of an individual's deficiencies is required. Thus, additional methods of diagnosing and supplementing nutritional deficiencies are required.

### SUMMARY

In one aspect, provided herein are methods for providing a nutritional supplement for a subject, the method comprising:
i. obtaining or having obtained levels of a plurality of extracellular nutrients measured from serum obtained from the subject;
ii. obtaining or having obtained levels of a plurality of intracellular nutrients measured from intracellular white blood cells (WBCs) and/or red blood cells (RBCs) obtained from the subject;
iii. determining a personalized dosage of the nutritional supplement for the subject using both of the levels of the plurality of extracellular nutrients and the levels of the plurality of intracellular nutrients.

In some embodiments, the extracellular nutrients comprise one or more of vitamins, minerals, amino acids, and fatty acids.

In some embodiments, the intracellular nutrients comprise one or more of vitamins, minerals, amino acids, and fatty acids.

In some embodiments, both the extracellular nutrients and the intracellular nutrients each comprise one or more of vitamins, minerals, amino acids, and fatty acids.

In some embodiments, the vitamins comprise one or more of Vitamin A (Retinol), Vitamin A (beta-carotene), Vitamin B1 (Thiamine diphosphate), Vitamin B2 (Riboflavin 5-Phosphate), Vitamin B3 (Nicotinic acid), Vitamin B5 (Pantothenic acid), Vitamin B6, Pyridoxal 5-Phosphate, Vitamin B7 (Biotin), Vitamin B12 (Cyanocobalamin), Vitamin C (L-Ascorbic acid), Vitamin D, 25-OH, Vitamin D3 (Cholecalciferol), Vitamin D, 1-25 dihydroxy, Vitamin E (alpha-tocopherol), Vitamin K1 (Phylloquinone), Vitamin K2 (Menaquinone-MK-7), Folate (L-5-methyltetrahydrofolate), and Coenzyme Q10 (Ubiquinone+Ubiquinol).

In some embodiments, the minerals comprise one or more of Selenium, Sodium, Potassium, Calcium, Zinc, Manganese, Iron, Magnesium, Copper, Chromium, Myo-Inositol, Iodine, Molybdenum, Phosphorus, Tetrahydrobiopterin, Fluoride, and Copper/Zinc.

In some embodiments, the amino acids comprise one or more of Glutathione Oxidized, MMA (Methylmalonic acid), Choline, L-Cysteine, L-Asparagine, L-Glutamine, L-Serine, L-Arginine, L-Citrulline, L-Isoleucine, L-Valine, L-Leucine, Free Carnitine, and Phenylalanine.

In some embodiments, the fatty acids comprise one or more of DHA (Docosahexaenoic acid), EPA (Eicosapentaenoic acid), DPA (Docosapentaenoic acid), AA (Arachidonic acid), LA (Linoleic acid), Omega-3 Total, Omega-6 Total, Omega-3 Index, and AA/EPA.

In some embodiments, determining the personalized dosage of the nutritional supplement comprises, for one of an extracellular nutrient or an intracellular nutrient, determining a ratio of a target nutrient value and the obtained level of the extracellular nutrient or intracellular nutrient.

In some embodiments, determining the personalized dosage of the nutritional supplement comprises:
i. for an extracellular nutrient, determining a ratio of a target extracellular nutrient value and the obtained level of the extracellular nutrient; and
ii. for an intracellular nutrient, determining a ratio of a target intracellular nutrient value and the obtained level of the intracellular nutrient.

In some embodiments, the method further comprises obtaining or having obtained genetic information of the subject, the genetic information relating to inhibition or absorption of nutrients.

In some embodiments, the genetic information of the subject comprises genetic statuses of one or more genomic locations of a plurality of genes.

In some embodiments, the genetic statuses comprise presence or absence of a polymorphism.

In some embodiments, the polymorphism is one of rs12934922, rs6564851, rs7501331, rs11645428, rs11645428, rs10766197, rs10741657, rs10877012, rs1801131, rs1801133, rs7946, rs174547, rs17514104, rs492602, rs602662, rs526934, rs33972313, rs4257763, rs6139591, rs6596473, rs2304478, rs889299, rs4516035, rs11126936, rs13107325, rs1799945, rs1800562, rs76151636, rs4074995, rs12785878, rs1799983, rs13078881, rs2108622, rs1050450, rs4680, rs225014, rs594445, rs4284505, rs1695, rs291466, rs121918252, rs2282679, rs12272004, rs3877899, rs4588, rs4820268, rs855791, rs775607037, rs786204770, rs8007267, rs121909307, rs3733890, rs7204044, rs1667255, rs3811647, and rs5030853.

In some embodiments, the plurality of genes comprises one or more of BCMO1, CYP2R1, MTHFR, PEMT, FADS1, SLC35F3, FUT2, TCN1, SLC23A1, SLC23A2, SLC12A3, SCNN1B, VDR, SLC30A3, SLC39A8, HFE, ATP7B, RGS14, NADSYN1, NOS3, BTD, CYP4F2, GPX1, COMT, DI02, MOCOS, ESR1, GSTP1, HICBH, MUT, GC, APOA5, SEPP1, TF, VDR, TMPRSS6, COQ4, PAH, GCH1, GSS, BHMT, and PAH.

In some embodiments, the method further comprises determining a polygenic risk factor according to the genetic statuses of the one or more locations of the plurality of genes.

In some embodiments, determining the personalized dosage further comprises determining the personalized dosage of the nutritional supplement for the subject according to the polygenic risk score.

In some embodiments, determining a polygenic risk factor according to the genetic statuses of the one or more locations of the plurality of genes comprises for each of the one or more locations of a gene, assigning a genotype score according to presence or absence of a polymorphism at the location of the gene; and combining the genotype scores across the locations of the plurality of genes.

In some embodiments, the method further comprises obtaining or having obtained body metrics of the subject.

In some embodiments, the body metrics of the subject comprise one or more of height and weight.

In some embodiments, determining the personalized dosage further comprises determining the personalized dosage of the nutritional supplement according to the body metrics of the subject.

In some embodiments, the method further comprises obtaining or having obtained an absorption factor determined for the subject, the absorption factor reflecting subject's utilization of a plurality of nutrients.

In some embodiments, determining the personalized dosage further comprises determining the personalized dosage of the nutritional supplement for the subject according to the absorption factor.

In some embodiments, the absorption factor for the subject is determined by comparing pre-supplementation blood nutrient values to post-supplementation blood nutrient values.

In some embodiments, comparing pre-supplementation blood nutrient values to post-supplementation blood nutrient values comprises determining a difference between the pre-supplementation and post-supplementation blood nutrient values.

In some embodiments, the pre-supplementation blood nutrient values are determined from a blood sample obtained from the subject prior to providing a supplement.

In some embodiments, the post-supplementation blood nutrient values are determined from a blood sample obtained from the subject subsequent to providing a supplement.

In some embodiments, the supplement comprises one or more of Vitamin A Palmitate, Beta Carotene, Thiamine Mononitrate, Riboflavin 5 Phosphate, Nicotinic Acid, Calcium Pantothenate, Pyridoxine HCl, Biotin, Cyanocobalamin, Ascorbic Acid, Cholecalciferol, d-Alpha Tocopheryl Succinate, Vitamin K1, Vitamin K2 as Menaquinone-7), Folinic Acid (Folate), Ubiquinone, L-Selenomethionine, Sea Salt, Potassium chloride, Calcium carbonate, Zinc Picolinate, Manganese Glycinate, Ferrous sulfate, Magnesium Citrate, Copper Bisglycinate Chelate, Chromium picolinate, Myo-inositol, Potassium Iodide, Molybdenum Glycinate Chelate, Dipotassium phosphate, L-Glutathione (reduced), Cyanocobalamin, Choline bitartrate, N-Acetyl-L-Cysteine (NAC), L-Asparagine, L-Glutamine, L-Serine, L-Arginine, L-Citrulline, L-Isoleucine, L-Valine, L-Leucine, L-carnitine, L-Phenylalanine, DHA (Docosahexaenoic acid), EPA (Eicosapentaenoic acid), Arachidonic Acid, Conjugated Linoleic Acid, and Omega-3 DHA/EPA (High DHA) 3:1.

In some embodiments, the method further comprises obtaining a recommended dietary allowance (RDA) value for the subject, wherein the RDA value is determined according to the subject's age, gender, or pregnancy or lactation status.

In some embodiments, determining the personalized dosage further comprises determining the personalized dosage of the nutritional supplement for the subject according to the RDA value.

In some embodiments, the method further comprises obtaining or having obtained a measure of the subject's gut microbiome.

In some embodiments, determining the personalized dosage further comprises determining the amount of probiotics or prebiotics of the nutritional supplement for the subject according to the subject's gut microbiome.

In some embodiments, the measure of the subject's gut microbiome comprises levels of one or more of viruses, fungi, parasites, and worms.

In some embodiments, the measure of the subject's gut microbiome is obtained by performing a Gut Zoomer™ assay.

In some embodiments, the method further comprises:
i. obtaining or having obtained one or more of:
ii. biomarkers of the subject indicative of cardiovascular health;
iii. biomarkers of the subject indicative of neurological health;
iv. biomarkers of the subject indicative of thyroid health;
v. biomarkers of the subject indicative of kidney health; and
vi. biomarkers of the subject indicative of liver health.

In some embodiments, determining the personalized dosage further comprises determining the personalized dosage of the nutritional supplement for the subject according to the one or more of:
i. levels of nutrients and/or supporting supplements supportive of cardiovascular health;
ii. levels of nutrients and/or supporting supplements supportive of neurological health;
iii. levels of nutrients and/or supporting supplements supportive of thyroid health;
iv. levels of nutrients and/or supporting supplements supportive of kidney health; and
v. levels of nutrients and/or supporting supplements supportive of liver health.

In some embodiments, the levels of nutrients of the subject indicative of cardiovascular health comprise levels of one or more of L-arginine and L-citrulline.

In some embodiments, the levels of nutrients of the subject indicative of neurological health comprise levels of one or more of folate, vitamin E, and omega-3 fatty acids.

In some embodiments, the levels of nutrients of the subject indicative of thyroid health comprise levels of one or more of iodine, selenium, and zinc.

In some embodiments, the levels of nutrients of the subject indicative of kidney health comprise levels of one or more of vitamin B6 and EPA (eicosapentaenoic acid).

In some embodiments, the levels of nutrients of the subject indicative of liver health comprise levels of zinc.

In some embodiments, the method further comprises obtaining or having obtained one or more responses from the subject via a patient questionnaire.

In some embodiments, the patient questionnaire comprises one or more questions related to patient medical history, gender, height, weight, nutrient deficiencies, and health goals.

In some embodiments, the nutritional supplement comprises one or more supplements that are selected based on the one or more responses from the subject via the patient questionnaire.

In some embodiments, the one or more supplements comprise any of Hydroxocobalamine, Methylcobalamin, L-5-Methyltetrahydrofolate, Folic acid, L-Carnitine tartrate, Magnesium L-threonate, L-5-methyltetrahydrofolate, calcium salt, Zinc gluconate, Inositol hexanicotinate, Zinc sulfate, Magnesium taurate, Mixed tocopherols, Ferrous bisglycinate chelate, Magnesium malate, Sodium ascorbate, Zinc carnosine, Potassium citrate, Calcium citrate, Vitamin B12, Citrulline, Vitamin D, L-Isoleucine, L-Valine, L-Leucine, L-Arginine, Taurine, Vitamin C, Vitamin E, Beta-carotene, Selenium, Coenzyme Q10, Manganese, Beta-alanine, Lysine, L-valine, Methionine, Phenylalanine, Threonine, Tryptophan, Histidine, Glycine, Vitamin D3, DHA (Docosahexaenoic acid), EPA (Eicosapentaenoic acid), L-methionine, and L-glutamine.

In some embodiments, the one or more supplements are selected based on one or more responses as shown in Table 6.

In some embodiments, the nutritional supplement further comprises one or more supporting supplements.

In some embodiments, the one or more supporting supplements are selected based on the one or more supplements included in the nutritional supplement.

In some embodiments, the one or more supporting supplements are selected based on one or more supplements as shown in Table 6.

In some embodiments, the one or more supporting supplements are selected based on presence of one or more polymorphisms for the subject at one or more genomic locations of a plurality of genes.

In some embodiments, the one or more supporting supplements are selected based on presence of one or more polymorphisms for the subject as shown in Table 8.

In some embodiments, the one or more supporting supplements comprise any of Micro PQQ, Ginger, Curcumin, Berberine extract, Phosphatidylcholine, Quercetin, Phosphatidylserine, Licorice, Broccoli, Green tea extract, 5-hydroxy-tryptophan, Nitrates, Caffeine, Probiotics, Prebiotics, Epigallocatechin gallate, Ginseng, Rhodiola rosea, β-hydroxy-β-methylbutyrate, α-ketoisocaproic acid, Methylsulfonyl-methane, Betaine, Silymarin, Resveratrol, Lycopene, Catechin, Chitosan, and Glucoraphanin.

In some embodiments, the method further comprises administering or having administered the personalized dosage of the nutritional supplement to the subject.

In some embodiments, the method is repeated at least one, two, three, four, five, six, seven, eight, nine, ten, or more times.

In some embodiments, the method further comprises periodically obtaining or having obtained post-supplementation blood nutrient values after that the personalized dosage of the nutritional supplement has been administered to the subject; and determining whether to modify the personalized dosage of the nutritional supplement based on the post-supplementation blood nutrient values.

In some embodiments, the personalized dosage of the nutritional supplement is changed after the at least one, two, three, four, five, six, seven, eight, nine, ten, or more repeats.

In some embodiments, the nutritional supplement comprises a combination of any of:
i. calcium and vitamin D;
ii. magnesium and vitamin D;
iii. omega 3 and vitamin E;
iv. sodium and potassium;
v. folate and vitamin B12;
vi. vitamin B3 and tryptophan;
vii. vitamin D and omega 3; and
viii. vitamin C and iron.

In some embodiments, the nutritional supplement does not include a combination of any of:
i. iron and zinc;
ii. zinc and magnesium;
iii. copper and zinc;
iv. calcium and iron; and
v. vitamin E and vitamin K.

In some embodiments, the nutritional supplement is provided as a powder, capsule, tablet, or emulsion, or any combination thereof.

In some embodiments, the nutritional supplement further comprises a flavoring agent.

In some embodiments, before obtaining or having obtained levels of a plurality of extracellular nutrients measured from serum and/or intracellular nutrients measure from white blood cells (WBCs) and/or red blood cells (RBCs) obtained from the subject, the method further comprises administering an Absorption Testing Blend (ATB) to the subject for a first predefined period.

In some embodiments, the ATB comprises a multi-nutrient blend comprised of vitamins, minerals, amino acids and fatty acids.

In some embodiments, the vitamins, minerals, amino acids and fatty acids are in dosages close to the RDA values or the safe or generally advised values for nutrients.

In some embodiments, the ATB is the same across various patients

In some embodiments, the ATB is further adjusted with gut microbiome correction before being administered to the subject.

In some embodiments, the method further comprises obtaining or having obtained pre-supplementation blood nutrient values and post-supplementation blood nutrient values, wherein the pre-supplementation blood nutrient values are determined before administering the ATB to subject and the post-supplementation blood nutrient values are determined after administering the ATB for the first predefined period.

In some embodiments, determining a personalized dosage of the nutritional supplement comprises determining a vibrant absorption factor (VAF) based on a difference between pre-supplementation nutrient values and post-supplementation nutrient values, divided by an ATB dosage; determining a target nutrient value (TNV) for each nutrient in both cellular and serum contexts; and determining a personalized dosage for each nutrient based on a difference between the TNV and the post-supplementation nutrient values, divided by the VAF.

In some embodiments, the method further comprises producing the nutritional supplement for the subject, the nutritional supplement personalized for the subject by containing the personalized dosages for each nutrient.

In some embodiments, the method further comprises adjusting the determined personalized dosage of the nutritional supplement by using nutrient pairing.

In some embodiments, the method further comprises dividing nutrient reference range for individuals into a number of zones with different nutrient wellness levels.

In some embodiments, the number of zones comprises 3 or more, 4 or more, 5 or more, 6 or more, 7 or more, 8 or more, 9 or more, or 10 or more, or another number of zones.

In some embodiments, the number of zones comprises five zones.

In some embodiments, determining a personalized dosage of the nutritional supplement for the subject comprises determining a current zone of a nutrient for the subject using both of the levels of the plurality of extracellular nutrients and the levels of the plurality of intracellular nutrients; determining a target nutrient zone for the nutrient; and determining a multiplication factor for the nutrient based on the current zone and the target nutrient zone for the nutrient; and determine the personalized dosage of the nutritional supplement for the subject based on the multiplication factor for the nutrient.

In some embodiments, determining the personalized dosage of the nutritional supplement for the subject based on the multiplication factor comprises multiplying a starting dosage for the nutrient with the multiplication factor.

In some embodiments, the determined current zone for the nutrient is adjusted based on nutrient pairing associated with the nutrient.

In some embodiments, the determined current zone for the nutrient is adjusted based on health comorbidities associated with the nutrient.

In some embodiments, the determined current zone for the nutrient is adjusted based on genetic predispositions of the subject.

In some embodiments, the method further comprises administering the personalized dosage of the nutritional supplement to the subject with one or more predefined supplements.

In some embodiments, the one or more predefined supplements comprise one or more of supporting supplements.

In some embodiments, the one or more predefined supplements comprise one or more probiotic supplements.

In some embodiments, the one or more predefined supplements comprise one or more of folate, calcium, vitamin E, iron, vitamin K, L-carnitine, tartrate, quercetin, phosphatidylserine, vitamin B3, DHA (Docosahexaenoic acid), curcumin, broccoli, or Lactobacillus reuteri.

In some embodiments, the one or more predefined supplements are administered to the subject in separate morning dose and evening dose.

### BRIEF DESCRIPTION OF THE SEVERAL VIEWS OF THE DRAWINGS

These and other features, aspects, and advantages of the present disclosure will become better understood with regard to the following description, and accompanying drawings, where:

FIG. 1 provides a schematic diagram of the process of the NutriPro test from the individual subject to the creation of customized nutritional blends.

FIG. 2 provides a diagram showing the information used to determine the customized nutritional dosage for optimized nutrition.

FIGS. 3A-3C provide flowcharts of various exemplary methods for Absorption Testing Blend-based approach to optimizing nutrient levels.

FIGS. 4A-4C provide flowcharts of various exemplary methods for Zone-based approach to optimizing nutrient levels.

### DETAILED DESCRIPTION

### Definitions

Terms used in the claims and specification are defined as set forth below unless otherwise specified.

The term “ameliorating” refers to any therapeutically beneficial result in the treatment of a disease state, e.g., a nutritional deficiency or disease state associated with a nutritional deficiency, including prophylaxis, lessening in the severity or progression, remission, or cure thereof.

The term “in situ” refers to processes that occur in a living cell growing separate from a living organism, e.g., growing in tissue culture.

The term “in vivo” refers to processes that occur in a living organism.

The term “mammal” as used herein includes both humans and non-humans and include but is not limited to humans, non-human primates, canines, felines, murines, bovines, equines, and porcines.

The term “sufficient amount” means an amount sufficient to produce a desired effect, e.g., an amount sufficient to modulate protein aggregation in a cell.

The term “therapeutically effective amount” is an amount that is effective to ameliorate a symptom of a disease. In some embodiments, a therapeutically effective amount can be a “prophylactically effective amount” as prophylaxis can be considered therapy.

Abbreviations used in this application include the following:

It must be noted that, as used in the specification and the appended claims, the singular forms “a,” “an” and “the” include plural referents unless the context clearly dictates otherwise.

### Methods

The present disclosure relates to the field of nutrition, particularly the effective diagnosis of nutrient deficiencies followed by effective nutritional supplementation via a supplement blend that is customized to meet the needs of an individual's nutrient requirements. The supplement blend also takes into account the genetics, extracellular assessment values (serum nutrient levels), intracellular assessment values (RBC and WBC nutrient levels), microbiome assessment (gut health), absorption factor, body metrics, nutrient metrics, symptoms, conditions, comorbidities, and supporting health factors that will aid in a holistic approach to treating nutrient deficiencies.

Provided herein are methods of analyzing nutrient levels in serum in combination with assessing intracellular nutrient levels in RBCs and WBCs. Such intracellular nutrient levels can be indicative of cellular uptake, utilization, and the presence of nutrients in the body over a period of time. In some aspects, the methods further comprise genetic assessment of any predispositions which may directly affect nutrient levels or processes influencing nutrient levels in the body. The combination of genetic testing, extracellular, and/or intracellular testing provides a fuller assessment of nutrient levels and the processes affecting the same in the body.

The method for providing a nutritional supplement for a subject as disclosed herein is based on a comprehensive panel that assesses intracellular and extracellular levels of at least or up to 41 nutrients. This analysis allows for a more effective diagnosis of a subject's underlying nutrient deficiency. Blood analysis using the method disclosed herein tests for nutrient levels in serum, red blood cells (RBCs), and white blood cells (WBCs). Additional genetic testing can assess patients' predispositions toward nutrient deficiencies. Based on the blood and genetic analysis results, a subsequent supplementation diet comprising supplements and food sources can be designed and implemented. The subject can be a human subject.

In some aspects, provided herein are nutrient detection methods that evaluate a subject's comprehensive nutrient levels. In some embodiments, the detection method assesses nutrients both in extracellular plasma and intracellular in RBC and WBC. In some embodiments, the detection method is mass spectroscopy-based. The extracellular and intracellular nutrient levels provide a comprehensive analysis of a subject's nutrient status, reflecting not only the intake levels but also the absorption levels into the cells.

In some aspects, the extracellular nutrient levels provide a snapshot of the status of nutrient baseline levels at a given time. It is a reflection of a person's diet over a narrow period of time. In contrast, the intracellular levels of nutrients provide information on the absorbed levels of nutrients, thus accounting for factors such as aging, lifestyle, chronic illness, medication, etc. that could interfere with absorption and can change the functional nutritional levels. Additionally, the test panel can also test for nutrient deficiency predispositions at the genetic level. Genetic testing for predispositions provides information about a patient's susceptibility to nutrient deficiencies.

The comprehensive panel can also assess nutrient deficiencies at the genetic level to assess the scope of predispositions that may result in various nutrient deficiencies. Genetic testing can be carried out using reverse transcription polymerase chain reaction (RT-PCR), quantitative PCR (qPCR) or any other nucleic acid detection method known in the art.

In some aspects, the method for providing a nutritional supplement disclosed herein can be used to identify nutrient deficiencies and design personalized supplementation diets comprising supplements and nutrient-rich food sources. The method for providing a nutritional supplement disclosed herein can subsequently establish the effectiveness of the given diet and supplement plan by monitoring the improvement in patients' symptoms. In some embodiments, the subjects can undergo additional testing to optimize the nutritional needs of the subject. Such testing can be performed every 2 months, 3 months, 4 months, 5 months, 6 months, 7 months, 8 months, 9 months, 10 months, 11 months, or yearly to modulate the levels of nutrients needed to restore nutritional health continually in a subject.

### Nutritional Deficiency Testing

In one aspect, provided herein are methods of assessing nutrient levels extracellularly and/or intracellularly in a subject. In some embodiments, the nutrient values (e.g., levels) are blood nutrient values. In some embodiments, the blood nutrient values are extracellular values (e.g., serum levels) and/or intracellular values (e.g., red blood cell (RBC) and/or white blood cell (WBC) values). In some embodiments, a “nutrient level” and “nutrient value” are interchangeable terms.

In one aspect, provided herein are methods for providing a nutritional supplement for a subject, the method comprising: obtaining or having obtained levels of a plurality of extracellular nutrients measured from serum obtained from the subject; obtaining or having obtained levels of a plurality of intracellular nutrients measured from intracellular white blood cells (WBCs) and/or red blood cells (RBCs) obtained from the subject; and determining a personalized dosage of the nutritional supplement for the subject using both of the levels of the plurality of extracellular nutrients and the levels of the plurality of intracellular nutrients.

In some embodiments, the extracellular nutrients comprise one or more of vitamins, minerals, amino acids, and fatty acids. In some embodiments, the intracellular nutrients comprise each of vitamins, minerals, amino acids, and fatty acids. In some embodiments, both the extracellular nutrients and the intracellular nutrients each comprise each of vitamins, minerals, amino acids, and fatty acids.

In some embodiments, the vitamins comprise one or more of Vitamin A (Retinol), Vitamin A (beta-carotene), Vitamin B1 (Thiamine diphosphate), Vitamin B2 (Riboflavin 5-Phosphate), Vitamin B3 (Nicotinic acid), Vitamin B5 (Pantothenic acid), Vitamin B6, Pyridoxal 5-Phosphate, Vitamin B7 (Biotin), Vitamin B12 (Cyanocobalamin), Vitamin C (L-Ascorbic acid), Vitamin D, 25-OH, Vitamin D3 (Cholecalciferol), Vitamin D, 1-25 dihydroxy, Vitamin E (alpha-tocopherol), Vitamin K1 (Phylloquinone), Vitamin K2 (Menaquinone-MK-7), Folate (L-5-methyltetrahydrofolate), and Coenzyme Q10 (Ubiquinone+Ubiquinol).

In some embodiments, the minerals comprise one or more of Selenium, Sodium, Potassium, Calcium, Zinc, Manganese, Iron, Magnesium, Copper, Chromium, Myo-Inositol, Iodine, Molybdenum, Phosphorus, Tetrahydrobiopterin, Fluoride, and Copper/Zinc.

the amino acids comprise one or more of Glutathione Oxidized, MMA (Methylmalonic acid), Choline, L-Cysteine, L-Asparagine, L-Glutamine, L-Serine, L-Arginine, L-Citrulline, L-Isoleucine, L-Valine, L-Leucine, Free Carnitine, and Phenylalanine.

In some embodiments, the fatty acids comprise one or more of DHA (Docosahexaenoic acid), EPA (Eicosapentaenoic acid), DPA (Docosapentaenoic acid), AA (Arachidonic acid), LA (Linoleic acid), Omega-3 Total, Omega-6 Total, Omega-3 Index, and AA/EPA.

In some embodiments, the one or more of vitamins, minerals, amino acids, and fatty acids are selected from Table 1. In some embodiments, the genetic testing for the one or more of vitamins, minerals, amino acids, and fatty acids deficiencies are selected from Table 1.

Table 1 provides an exemplary set of nutrients that can be tested, as well as the tissue source (serum, RBC, or WBC). Table 1 also provides exemplary genetic testing for each vitamin and mineral deficiency.

In some embodiments, the method provided herein assesses the nutrient levels in subject samples via any protein detection method known in the art. In some embodiments, the detection method is mass spectrometry. In some embodiments, the detection method is Liquid chromatography-mass spectrometry (LC-MS), Gas chromatography-mass spectrometry (GC-MS), or Inductively coupled plasma-mass-spectrometry (ICP-MS). In some embodiments, the detection method is Liquid chromatography-mass spectrometry (LC-MS). In some embodiments, the detection method is Gas chromatography-mass spectrometry (GC-MS). In some embodiments, the detection method is Inductively coupled plasma-mass-spectrometry (ICP-MS).

In some embodiments, the method provided herein evaluates nutrient levels, both in extracellular serum and inside (intracellular) red blood cells (RBC) and/or white blood cells (WBC). The extracellular nutrient levels provide a snapshot of the status of nutrient baseline levels at a given time. These extracellular nutrient levels are a reflection of a person's diet over a narrow period of time. In contrast, RBCs have a normal lifespan of 120 days, and thus intracellular assessments for nutrients in RBCs can provide information on nutrients over longer periods. Additionally, the intracellular levels of nutrients provide information on the absorbed levels of nutrients, thus accounting for factors such as, but not limited to, aging, lifestyle, chronic illness, and medication, that can interfere with absorption and can change the functional nutritional levels. Moreover, actual nutrient utilization occurs inside the cell owing to cellular metabolic processes. At times, nutrients could be present in serum, but poor cellular uptake may result in cells being devoid of these nutrients, thus giving rise to nutrient deficiencies owing to affected cellular activities. For example, poor cellular uptake of vitamin B1 via the thiamine transporter protein can give rise to vitamin B1 deficiency irrespective of its prevalence in serum. Similarly, the affected uptake of vitamin C via the vitamin C transporter protein results in the reduced active transport of vitamin C across the cell membrane which can give rise to a vitamin C deficiency as the cells are not able to get appropriate levels of vitamin C for their function. As a result, it is important to assess nutrients intracellularly in addition to extracellularly. Thus, the intracellular assessment provides information on cellular uptake and utilization of nutrients at the most fundamental level, enabling understanding of the root cause of nutrient deficiencies.

In some embodiments, determining the personalized dosage of the nutritional supplement comprises, for one of an extracellular nutrient or an intracellular nutrient, determining a ratio of a target nutrient value and the obtained level of the extracellular nutrient or intracellular nutrient.

In some embodiments, determining the personalized dosage of the nutritional supplement comprises: for an extracellular nutrient, determining a ratio of a target extracellular nutrient value and the obtained level of the extracellular nutrient; and for an intracellular nutrient, determining a ratio of a target intracellular nutrient value and the obtained level of the intracellular nutrient.

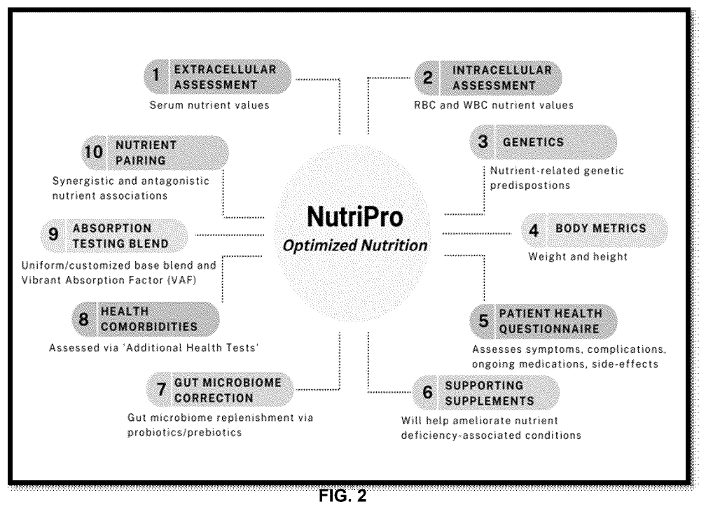

FIG. 2 provides an overview of the various aspects of the nutritional testing and treatments provided by the method and compositions disclosed herein. The nutritional dosage form accounts for extracellular (1) and intracellular (2) nutrient values, the nutrient-related genetic predispositions (3), the subject's body metrics (4), and vibrant absorption factor (9); the patient health questionnaire (PHQ) (5) assess symptoms, complications, ongoing medications, and side-effects in order to ascertain the best form of nutrients, as well as appropriate supporting supplements to help ameliorate nutrient deficiency-associated conditions (6). Additionally, genetics via the PRF (3) are also considered when determining appropriate supporting supplements (6). The ‘Gut Microbiome Correction’ (7) can be carried out via the ‘Gut Zoomer’ test. Similarly, test-based health comorbidities (8) can be used to guide incorporation of nutrients and supporting supplements (6) into the blend. Nutrient pairings (10) based on biological technical considerations, can also be considered while creating the blend. In some embodiments, the ‘NutriPro Dosage Formula’ accounts for all the 10 aspects shown in FIG. 2.

### Nutrient Metrics

In some embodiments, nutrient metrics refer to the ‘target’ and the ‘actual’ nutrient values. Such values can be used in determining the dosage formula. The dosage formula can be used to determine the dosage needed to optimize the subject's nutrients. The “Target Nutrient Value” (TNV) for the extracellular (serum) and intracellular (RBC and WBC) components can be the 90th percentile of the reference range provided in Table 2. Table 2 provides exemplary optimum nutrient ranges and 90th percentiles of the optimum ranges for serum, RBC, and WBC nutrient values of the indicated vitamins and minerals. The “Actual Nutrient Values” (ANV) can be the actual nutrient levels observed after blood analysis of the extracellular (serum) and intracellular components. In some embodiments, the TVN is selected from the nutrient reference range for each indicated nutrient provided in Table 2. In some embodiments, the TVN is selected from the 90^{th} percentile of the nutrient reference range for each indicated nutrient provided in Table 2.

In some embodiments, a physician can modulate the TNV in the dosage formula according to the individual's needs. For example, an athlete's nutrient requirements may be higher, thus increasing the TNV.

In one embodiment, the nutrient forms in the supplement blend are modified according to the individual's nutrient deficiency-associated symptoms. For example, various forms of vitamin B12 are present for different kinds of anemia (pernicious or macrocytic), which is one of the major symptoms associated with vitamin B12 deficiency.

In one embodiment, the nutrient forms in the supplement blend are modified according to the individual's pre-existing conditions. For example, individuals with gastrointestinal (GI) conditions can be given suitable forms of iron and vitamin C that cause less GI discomfort. As another example, individuals suffering from blood pressure, memory, and cognitive issues will be given suitable magnesium forms accordingly.

In one embodiment, the nutrient forms in the supplement blend can be modified according to the individual's other medications. For example, for individuals taking medications such as acid-reducing heartburn medications, the appropriate and compatible form of calcium can be given.

### Genetic Testing

In some aspects, provided herein are methods for determining nutrient deficiency predispositions at the genetic level (e.g., single nucleotide polymorphisms (SNPs), or other genetic mutations). Genetic testing for predispositions can assist in providing information about a subject's susceptibility to nutrient deficiencies. Table 1 and Table 3 provide exemplary nutrient-related polymorphisms that can be assessed. SNPs can be identified in the Single Nucleotide Polymorphism (dbSNP) database managed by the National Library of Medicine at ncbi.nlm.nih.gov/snp/ as of Aug. 8, 2023. The dbSNP database contains the human single nucleotide variations, microsatellites, and small scale insertions and deletions, as well as genomic and RefSeq mapping information for common variations and clinical mutations.

Single nucleotide polymorphisms (SNPs) related to nutrients can either inhibit or promote the absorption or production of the nutrient in question which makes it a key component to be considered while diagnosing and treating nutrient deficiencies. There are various processes governed by specific genes that can affect the nutrient status in the body. Alterations in these genes may affect their normal functioning which can affect nutrient status. These alterations can be assessed through genetic testing. For example, polymorphisms in the gene encoding for the thiamine transporter protein that regulates the absorption of vitamin B1 give rise to a vitamin B1 deficiency. This deficiency can occur due to impaired vitamin B1 uptake into cells irrespective of its presence in serum. Thus, understanding the underlying cause of the deficiency enables the employment of effective measures of treatment for the same.

Polymorphisms related to nutrients can either inhibit or promote the absorption or production of the nutrient in question. Thus, genetic testing for polymorphisms or single nucleotide polymorphisms (SNPs) can be used to diagnose nutrient deficiencies for treatment. Genetic testing can be carried out using any appropriate method known in the art, including but not limited to, Reverse transcription polymerase chain reaction (RT-PCR) or quantitative-PCR (qPCR).

In some aspects, genetic testing can also aid in determining the best form of nutrient to be supplemented based on polymorphisms affecting biological pathways that impact nutrient levels. For example, several vitamin A polymorphisms in the BCMO1 gene can lead to reduced conversion of provitamin A carotenoids to vitamin A (form utilized by the body). This results in low levels of vitamin A in the body thus giving rise to a vitamin A deficiency. In such exemplary cases, subjects are be supplemented with the active form of vitamin A, retinol, which is readily used by the body, to optimize vitamin A levels. Similarly, mutations in the CYP2R1 gene which affects the conversion of vitamin D to its hydroxylated form, 25-hydroxyvitamin D (25(OH)D), and the CYP27B1 gene which further affects the conversion of vitamin D to its active form, 1,25-dihydroxyvitamin (or calcitriol) can lead to an overall vitamin D deficiency. In such exemplary embodiments, subjects with the CYP2R1 gene mutation can be supplemented with the hydroxylated form (25-hydroxyvitamin D) of vitamin D while individuals with the CYP27B gene mutation can be directly supplemented with calcitriol. These forms will not have to undergo the ‘conversion’ reactions and can be readily used by the body, which may actively help in increasing serum vitamin D levels.

In another embodiment, polymorphisms in the MTHFR gene, involved in the conversion of synthetic folic acid and dietary folate into its active form, L-methylfolate, affect how the body processes folate which can lead to its deficiency. Under such conditions, the methylated form of folate is suitable as it bypasses the conversion step mediated by MTHFR, thus being readily utilized by the body and improving serum levels of vitamin B9.

In another embodiment, Vitamin E in the blood is transported by a variety of lipoproteins. A mutation in the APOA5 gene responsible for lipid metabolism can in turn affect vitamin E levels, as they are carried by lipids. Similarly, mutations in the GC gene responsible for binding and transporting vitamin D metabolites to target tissues can result in reduced levels of vitamin D in the body. In these exemplary cases, the supplementation of vitamin E and D can be increased to increase the probability of vitamin E and D being picked up and transported via their respective transport molecules.

In another embodiment, polymorphisms can affect the ability of the thiamine transporter protein (encoded by the SLC35F3 gene) and vitamin C transporter protein (encoded by the SLC23A2 gene) to actively transport vitamin B1 and vitamin C across the cell membrane. This can give rise to deficiency-associated symptoms as the cells are devoid of vitamin B1 and vitamin C respectively. In order to correct the deficiencies, subjects can receive increased supplementation of vitamin B1 and vitamin C to increase the probability of these nutrients being taken up into the cell via their respective uptake proteins.

In another embodiment, mutations in the BTD gene leading to a deficiency in the enzyme whose functions involve the reuse and recycling of vitamin B7 can effectively alter vitamin B7 levels in the body. This polymorphism is associated with low levels of circulating vitamin B7 in the body. In this exemplary case, external supplementation of vitamin B7 can be administered so that the subject's cells can readily use it.

In another embodiment, polymorphisms in the COQ4 gene responsible for the production of coenzyme Q10 can lead to its deficiency. Thus, supplementation of coenzyme Q10 can be increased to optimize its levels in the body.

Therefore, genetic testing can aid in understanding the fundamental cause of altered nutrient values and can enable effective treatment for the same, such as by supplementing the individual with the appropriate form of the nutrient that will improve its levels. A summary of the effect of the exemplary nutrient-related polymorphisms assessed and exemplary supplements and approaches to correct the nutrient deficiencies are provided in Table 3.

Table 3 provides exemplary suitable supplement forms for various nutrient-related genetic polymorphisms.

In some embodiments, the method comprises obtaining or having obtained genetic information of the subject, the genetic information relating to inhibition or absorption of nutrients.

In some embodiments, the genetic information of the subject comprises genetic statuses of one or more genomic locations of a plurality of genes.

In some embodiments, the genetic statuses comprise presence or absence of a polymorphism. In some embodiments, the genetic statuses comprise presence or absence of a polymorphism in a gene. In some embodiments, the genetic statuses comprise presence or absence of one or more polymorphisms. In some embodiments, the genetic statuses comprise presence or absence of one or more polymorphisms in one or more genes. In some embodiments, the genetic statuses comprises presence or absence of two, three, four, five, six, seven, eight, nine, ten, or more polymorphisms. In some embodiments, the one or more polymorphisms and/or genes is selected from Table 3.

In some embodiments, the polymorphism is one or more of rs12934922, rs6564851, rs7501331, rs11645428, rs11645428, rs10766197, rs10741657, rs10877012, rs1801131, rs1801133, rs7946, rs174547, rs17514104, rs492602, rs602662, rs526934, rs33972313, rs4257763, rs6139591, rs6596473, rs2304478, rs889299, rs4516035, rs11126936, rs13107325, rs1799945, rs1800562, rs76151636, rs4074995, rs12785878, rs1799983, rs13078881, rs2108622, rs1050450, rs4680, rs225014, rs594445, rs4284505, rs1695, rs291466, rs121918252, rs2282679, rs12272004, rs3877899, rs4588, rs4820268, rs855791, rs775607037, rs786204770, rs8007267, rs121909307, rs3733890, rs7204044, rs1667255, rs3811647, and rs5030853.

In some embodiments, the plurality of genes comprises one or more of BCMO1, CYP2R1, MTHFR, PEMT, FADS1, SLC35F3, FUT2, TCN1, SLC23A1, SLC23A2, SLC12A3, SCNN1B, VDR, SLC30A3, SLC39A8, HFE, ATP7B, RGS14, NADSYN1, NOS3, BTD, CYP4F2, GPX1, COMT, DIO2, MOCOS, ESR1, GSTP1, HICBH, MUT, GC, APOA5, SEPP1, TF, VDR, TMPRSS6, COQ4, PAH, GCH1, GSS, BHMT, and PAH.

In some embodiments, the nutrient is selected from Table 3. In some embodiments, the general supplement is selected from Table 3. In some embodiments, the supplement is an alternative effective supplement selected from Table 3.

### Polygenic Risk Factors

The effect of nutrient-related genetic predispositions can also be considered and addressed in the method disclosed herein. The effect of exemplary alleles for the respective exemplary nutrient-related genetic polymorphism provided in Table 4 can be determined by calculating a genotype score for each subject. The genotype score can be based on the strength of each exemplary SNP that affects the nutrients levels in a subject. For example, when risk genotypes can be assigned a higher or a lower score depending on the risk associated with the given SNP based on the effect of the SNPs. For example, SNPs that are known to have more pronounced deleterious effects can be given a higher score.

In some aspects, for each nutrient, the cumulative effect of its SNPs can be calculated, resulting in a “Polygenic risk factor” (PRF). Exemplary PRFs for a given nutrient can be multiplied in the dosage formula to determine the dosage for the respective nutrient. Table 4 provides a summary of the genotype scores and the PRFs for the respective exemplary nutrient-related SNPs.

In some embodiments, the PRF can also be used to provide ‘supporting supplements’ based on the risk associated with the conditions caused nutrient-related SNPs (see Table 8).

In one embodiment, the PHQ determines the supporting supplements required for certain conditions and the PRF determines the supporting supplements required for conditions brought about nutrient-related SNPs. In one embodiment, the PRF further and also determines amount of recommended supporting supplements required for conditions brought about nutrient-related SNPs (Table 4).

In one embodiment, the nutrient forms in the supplement blend are modified according to the individual's genetics. For example, if an individual with a polymorphism that affects the function of the gene that converts beta-carotene to the active form of vitamin A, retinol, then the supplement blend can contain retinol which will readily be used by the body.

In some embodiments, the method further comprises determining a polygenic risk factor according to the genetic statuses of the one or more locations of the plurality of genes.

In some embodiments, determining the personalized dosage further comprises determining the personalized dosage of the nutritional supplement for the subject according to the polygenic risk score.

In some embodiments, determining a polygenic risk factor according to the genetic statuses of the one or more locations of the plurality of genes comprises: for each of the one or more locations of a gene, assigning a genotype score according to presence or absence of a polymorphism at the location of the gene; and combining the genotype scores across the locations of the plurality of genes.

In some embodiments, the nutrient is selected from Table 4. In some embodiments, the one or more polymorphism is selected from Table 4. For example, a risk factor can comprise testing for one, two, three, four, five, or more of the polymorphisms provided in Table 4.

### Body Metrics

The effect of body metrics or body composition can also be considered and addressed in the method disclosed herein. Body metrics or body composition can be a component that is calculated during nutrient supplementation. Changes in body metric factors such as height and weight can alter a subject's nutritional needs. Thus, body metrics can be factored during evaluation of the nutritional status of a subject, for determining supplementing nutrients, and for monitoring the progress of the implemented dietary/supplement interventions in the subject.

In some aspects, the individual's height and weight are compared to an ideal height and weight for the subject's given age. Ideal body weight and height can be based on charts for height-weight distribution based on gender. Exemplary charts can be found in Anthropometric Reference Data for Children and Adults: United States, 2015-2018, NIH Vital and Health Statistics Series 3, Number 46, or from the Continuous NHANES datasets provided at wwwn.cdc.gov/nchs/nhanes/ContinuousNhanes/Default.aspx, both with which are hereby incorporated by reference in their entirety. The following formula can be used to determine the Body Metrics factor:

$$Body Metrics = ( H \times W ) individual / ( H \times W ) ideal$$

In one embodiment, the nutrient forms in the supplement blend are modified according to the individual's demographics. For example, the supplement blend can take into account factors such as age, height, weight, and gender and pregnancy or lactating status.

In some embodiments, the method further comprises obtaining or having obtained body metrics of the subject.

In some embodiments, the body metrics of the subject comprise one or more of height and weight.

In some embodiments, determining the personalized dosage further comprises determining the personalized dosage of the nutritional supplement according to the body metrics of the subject.

### Questionnaire

In some aspects, a ‘Patient Health Questionnaire’ (PHQ) can be employed to query additional health data such as, but not limited to, symptoms, complications, comorbidities, ongoing medications, and side effects. This questionnaire can assist with the selection of suitable nutrient supplement forms to ameliorate certain symptoms and overall improve the subject's health. The health form may comprise the following questions: Gender; Height; Weight; Pregnant/Nursing; previous deficiencies in any nutrient; if previous deficiency was B12 then did the subject suffer from any of the following B12 deficiency symptoms: Macrocytic anemia, Leber optic atrophy, or High homocysteine levels; if previous deficiency was B29 then did the subject suffer from any of the following B29 deficiency symptoms: High homocysteine levels, Megaloblastic anemia, Macrocytic anemia; athlete status; poor sleep; difficulties in concentrating; suffer from depression, anxiety, or mood swings; suffer from flu or infections; experiencing pain in your bones or joints; sensitive skin; breakouts or pimples; excessive weight gain; high blood sugar levels; high blood pressure; heart conditions; gastrointestinal discomfort; renal conditions; liver conditions; currently taking any medications for heart conditions, hypertension, or acid reflux; energy slump after eating food; any regular exposure to the sun (10-15 min per day); request support for following fitness: endurance, muscle strength, muscle mass, muscles recovery.

The questionnaire can help determine the presence of complications or symptoms associated with altered nutrient levels, susceptibility to side effects that may be associated with nutrient supplementation, presence of comorbidities and their symptoms, or ongoing medication usage by the subject. The questionnaire can assess these aspects using direct questions which could be answered with either a ‘yes’ or a ‘no.’ For symptoms, a 10-point Likert scale can be provided to assess the severity of the symptoms. The severity can be taken into consideration, with more severe symptoms assigned a higher priority while creating the nutritional blend.

In exemplary cases where the symptom severity scores are tied, the default preference provided in Table 5 can be used to determine the priority condition. The PHQ responses can be used to determine the suitable forms of nutrients and the appropriate supporting supplements that apply to the condition/symptom/health complication in question.

The PHQ responses can determine alternate suitable forms of the nutrient to be administered to the subject. Thus, the PHQ can aid in customization to choose a more appropriate form over the ‘default form’ (default nutritional examples are provided in Table 16). Table 6 provides exemplary PHQ responses and the appropriate nutrient supplementation and supporting supplements.

For example, different forms of vitamin B12 are available to effectively treat different kinds of anemia (pernicious or macrocytic), which is one of the major symptoms associated with vitamin B12 deficiency. As vitamin B12 is an important cofactor used to convert homocysteine to methionine, low vitamin B12 levels result in high homocysteine levels which is a major risk factor for CVD. Methylcobalamin is the form of vitamin B12 that has been able to effectively reduce plasma homocysteine levels, which could be accompanied by a vitamin B12 deficiency. Similarly, folic acid is a good form to treat megaloblastic anemia which can be caused owing to a folate deficiency while L-5-Methyltetrahydrofolate can effectively increase serum folate levels and reduce homocysteine levels caused due to the MTHFR-gene mediated folate deficiency. L-methylfolate calcium is a good form of folate for individuals suffering from neuropsychiatric disorders.

In another example, individuals with gastrointestinal (GI) conditions can be given suitable forms of iron, magnesium, and zinc that cause less GI discomfort. Also, individuals suffering from blood pressure, memory, and cognitive issues can be given suitable magnesium forms accordingly. Various forms of zinc can accordingly be supplemented for immunity, skin, and GI concerns. Lastly, if the subject is taking medications such as acid-reducing heartburn medications, the appropriate and compatible form of calcium will be given.

In instances where a nutritional supplement is already being provided based on the extracellular and intracellular nutritional testing method and that nutrient is also identified via the PHQ, then the higher value among the ‘calculated dosage’ in the dosage formula and the RDA or generally advised value, can be considered while making the blend.

For example, in some embodiments, if an individual selects the option ‘no’ for the sun exposure question in the PHQ, then vitamin D and 5-hydroxy-tryptophan can be supplemented. In this example, since vitamin D is already a nutrient being determined by the method disclosed herein, the higher dosage among the calculated dosage and the RDA value can be selected. In contrast, 5-hydroxy-tryptophan which is not also being assessed will be incorporated based on its starting value as given in Table 7.

Thus, the combination of the nutrient level testing method and the PHQ can enable improving the individual's nutrient levels by supplementing with the best possible form of nutrients for other related or co-occurring conditions, thereby improving the patient's overall health.

Examples of the appropriate nutrient supplementation and supporting supplements for each PHQ response are provided in Table 6.

In some embodiments, the method further comprises obtaining or having obtained one or more responses from the subject via a patient questionnaire.

In some embodiments, the patient questionnaire comprises one or more questions related to patient medical history, gender, height, weight, nutrient deficiencies, and health goals.

In some embodiments, the nutritional supplement comprises one or more supplements that are selected based on the one or more responses from the subject via the patient questionnaire.

In some embodiments, the one or more supplements comprise any of Hydroxocobalamine, Methylcobalamin, L-5-Methyltetrahydrofolate, Folic acid, L-Carnitine tartrate, Magnesium L-threonate, L-5-methyltetrahydrofolate, calcium salt, Zinc gluconate, Inositol hexanicotinate, Zinc sulfate, Magnesium taurate, Mixed tocopherols, Ferrous bisglycinate chelate, Magnesium malate, Sodium ascorbate, Zinc carnosine, Potassium citrate, Calcium citrate, Vitamin B12, Citrulline, Vitamin D, L-Isoleucine, L-Valine, L-Leucine, L-Arginine, Taurine, Vitamin C, Vitamin E, Beta-carotene, Selenium, Coenzyme Q10, Manganese, Beta-alanine, Lysine, L-valine, Methionine, Phenylalanine, Threonine, Tryptophan, Histidine, Glycine, Vitamin D3, DHA, EPA, L-methionine, and L-glutamine.

In some embodiments, the one or more supplements are selected based on one or more responses as shown in Table 6.

### Supporting Supplements

Supporting supplements can help ameliorate the conditions caused by nutrient deficiencies and can be incorporated into the blend. Exemplary supplements can be included in the nutrient blend based on genetic testing via the PRF (Table 4), the PHQ and the PHQ outcome (Table 6), as well as the ‘Additional Health Tests’ (Tables 10, 11, 13, 14). For example, bone health is compromised during a vitamin D and calcium deficiency. Thus, the supporting supplements, ginger and curcumin which help improve bone health can be incorporated into the blend. Similarly, a licorice supplement can be added to prevent the deterioration of tooth health, which is generally associated with a fluoride deficiency. For example, and without wishing to be bound by theory, a fluoride deficiency can lead to tooth decay but excessive ingestion of fluoride is dangerous as it can cause dental fluorosis. As a result, simply providing a nutrient in excess may not be the best approach to address nutrient deficiency-related conditions because there is a limited range up to which the body can tolerate any given nutrient level. Thus, in such cases, supporting supplements can be beneficial by addressing the conditions caused by nutrient deficiencies. Exemplary various supporting supplements that can be incorporated into the blend for different health condition are summarized in Table 7.

In some embodiments, the nutritional supplement further comprises one or more supporting supplements.

In some embodiments, the one or more supporting supplements are selected based on the one or more supplements included in the nutritional supplement.

In some embodiments, the one or more supporting supplements are selected based on one or more supplements as shown in Table 6 or Table 7.

Supporting supplements can be an integral part of the PHQ. Several questions in the PHQ can lead to the incorporation of certain supporting supplements in the blend (Table 6). Additionally, the nutrient-related SNPs resulting in nutrient deficiencies can also ascertain the need for various supporting supplements which can improve the deficiency-related condition.

The score obtained in the PRF (Table 4) can determine the degree to which appropriate supporting supplements need to be recommended for a given SNP. Thus, the PRF will also account for the addition of supporting supplements based on the risk associated with the conditions brought about by the nutrient-related SNPs. However, the cumulative factor or the PRF can be multiplied for each supporting supplement.

Thus, while the PHQ can determine the supporting supplements required for certain conditions described in the questionnaire, the PRF obtained from genetic analysis can also determine the exemplary supporting supplements and the degree of recommendation based on any risk associated with SNPs. For information based on the PHQ which may determine only the appropriate nutrient with which to supplement the subject's diet, the supporting supplements can be supplemented in their general dosages as given in Table 7. The dosages have been determined based on extensive research in the field of nutrient supplementation.

In some embodiments, the one or more supporting supplements are selected based on presence of one or more polymorphisms for the subject at one or more genomic locations of a plurality of genes.

In some embodiments, the one or more supporting supplements are selected based on presence of one or more polymorphisms for the subject as shown in Table 8.

In some embodiments, the one or more supporting supplements comprise any of Micro PQQ, Ginger, Curcumin, Berberine Extract, Phosphatidylcholine, Quercetin, Phosphatidylserine, Licorice, Broccoli, Green tea extract, 5-hydroxy-tryptophan, Nitrates, Caffeine, Probiotics, Prebiotics, Epigallocatechin gallate, Ginseng, Rhodiola rosea, β-hydroxy-β-methylbutyrate, α-ketoisocaproic acid, Methylsulfonyl-methane, Betaine, Silymarin, Resveratrol, Lycopene, Catechin, Chitosan, and Glucoraphanin.

Table 8 summarizes the complete manner in which the supporting supplements can be incorporated into the blend based on conditions brought about by nutrient deficiency-related polymorphisms.

### Gut Microbiome Correction

The gut microbiome contributes to the nutrient pool. Gut bacteria can synthesize water-soluble B vitamins, such as biotin, cobalamin, folates, nicotinic acid, pantothenic acid, pyridoxine, riboflavin, and thiamine. Without wishing to be bound by theory, up to half of the daily vitamin K requirement in the body can be provided by gut bacteria. As a result, a decrease in the gut bacteria that synthesize the nutrient or an increase in the bacteria that restrict the synthesis of the nutrient, can result in the low intestinal synthesis of the given nutrient. Table 9 provides several nutrients along with their nutrient synthesizing-gut bacteria which could affect the respective nutrient levels.

In some aspects, the methods provided herein include an additional assessment of the gut microbiome for changes in gut bacterial species that may be altering nutrient levels. Such measurement thus enables correction of the gut microbiome via the supplementation of appropriate probiotics and prebiotics. For the gut microbiome correction, the ‘Gut zoomer’ test which assesses for various gut micro-organisms and metabolites can be conducted. This test uses microarray technology to assess multiple gut micro-organisms and gut metabolites simultaneously using stool samples. Gut microbiome tests are commercially available from a variety of vendors including, but not limited to, Vibrant Wellness (test code VAREQUISTION279).

The additional gut microbiome test disclosed herein is not limited to just gut commensal bacteria, but also assesses gut viruses, fungi, parasites, worms, and other gut inflammation and insufficiency biomarkers (e.g., gut entities/markers). In some embodiments, the appropriate supplements for altered levels of all gut entities/markers can be incorporated into the blend.

The gut microbiome results provide information for replenishing the altered microbial species via effective probiotic/prebiotic supplementation, thereby improving the nutrient levels associated with the altered level of the given microbial population. Table 9 provides exemplary nutrients, the gut commensals that synthesize these nutrients, and the prospective scope of correcting the gut population of the affected nutrient-synthesizing bacteria, which can improve gut-mediated altered nutrient levels. The gut microbiome assessment disclosed herein is not limited to the nutrients, bacteria, and exemplary corrective measures provided in Table 9.

In some embodiments, the supplementing nutrient is one or more nutrients provided in Table 9. In some embodiments, the bacteria is one or more bacteria provided in Table 9. In some embodiments, two, three, four, five, six, seven, eight, nine, ten or more bacteria as provided in Table 9 are assessed. In some embodiments, the probiotic/prebiotic supplementation is one or more exemplary corrective measure species provided in Table 9.

In some embodiments, the method further comprises obtaining or having obtained a measure of the subject's gut microbiome.

In some embodiments, determining the personalized dosage further comprises determining the amount of probiotics or prebiotics of the nutritional supplement for the subject according to the subject's gut microbiome.

In some embodiments, the measure of the subject's gut microbiome comprises levels of one or more of viruses, fungi, parasites, and worms.

In some embodiments, the measure of the subject's gut microbiome is obtained by performing a Gut Zoomer™ assay.

### Test-Based Health Comorbidities

Individuals may also be suffering from distinct health conditions that are present at the same time (comorbidities). In some aspects, the method described herein includes additional health tests to determine the appropriate nutritional supplementation for the individual. Exemplary health tests include, but are not limited to, cardiovascular health, neurological conditions, thyroid disease, kidney disease, and liver disease. The supplementation method disclosed herein can address these multiple health conditions via nutrient supplementation, thereby not only improving the individual's nutrient levels but also improving the individual's overall well-being by improving their comorbidities.

In some aspects, provided herein are additional health tests assessing one or more organ systems for comorbidities. The results from the tests can guide the inclusion of one or more appropriate nutrients to supplemented in the nutritional blend. Exemplary appropriate supplements for various biomarkers are also provided. Nutrient dosage formulas based on these biomarkers can be dependent on post-testing biomarker information.

In one embodiment, comorbidities such as heart conditions, hypertension, mild cognitive impairment, thyroid disease, etc. are ascertained using the ‘Additional Health Tests.’ In one embodiment, such comorbidities are considered while creating the ATB.

In some embodiments, the method further comprises obtaining or having obtained one or more of: biomarkers of the subject indicative of cardiovascular health; biomarkers of the subject indicative of neurological health; biomarkers of the subject indicative of thyroid health; biomarkers of the subject indicative of kidney health; and biomarkers of the subject indicative of liver health.

In some embodiments, determining the personalized dosage further comprises determining the personalized dosage of the nutritional supplement for the subject according to the one or more of: levels of nutrients and/or supporting supplements supportive of cardiovascular health; levels of nutrients and/or supporting supplements supportive of neurological health; levels of nutrients and/or supporting supplements supportive of thyroid health; levels of nutrients and/or supporting supplements supportive of kidney health; and levels of nutrients and/or supporting supplements supportive of liver health.

In some embodiments, the levels of nutrients of the subject indicative of cardiovascular health comprise levels of one or more of L-arginine and L-citrulline.

In some embodiments, the levels of nutrients of the subject indicative of neurological health comprise levels of one or more of folate, vitamin E, and omega-3 fatty acids.

In some embodiments, the levels of nutrients of the subject indicative of thyroid health comprise levels of one or more of iodine, selenium, and zinc.

In some embodiments, the levels of nutrients of the subject indicative of kidney health comprise levels of one or more of vitamin B6 and EPA.

In some embodiments, the levels of nutrients of the subject indicative of liver health comprise levels of zinc.

### Additional Health Tests:

In some embodiments, when exemplary nutrients (vitamins, minerals, amino acids, and fatty acids) that are being assessed by methods herein are supplemented due to the outcome of the ‘Additional Health Test’, then the higher value among the ‘calculated dosage’ of the i) dosage formula, ii) the RDA, or iii) the generally advised value (as provided in Table 15), can be considered when making the nutrient blend. Similarly, when the supporting supplements need to be supplemented, they can be incorporated based on their starting dosages as given in Table 7.

### Cardiovascular Health

Cardiovascular diseases (CVDs) include a variety of conditions that affect the heart, including but not limited to, infections, genetic defects, and blood vessel diseases. CVDs are highly prevalent among the masses and are crucial to consider when trying to optimize nutrient levels. In some aspects, the method provided herein comprises a further ‘Cardiovascular Health’ test which tests for biomarkers that are indicative of heart health, including, but not limited to, lipids, apolipoprotein, inflammation, myocardial stress, lipoproteins, and fatty acids. Cardiovascular disease tests that assess conditions such as lipid profiles, apolipoproteins, inflammation, myocardial stress, lipoprotein markers, and fatty acids, are commercially available from a variety of vendors including, but not limited to, Vibrant Wellness (vibrant-America.com/cardiovascular-health).

The results from the ‘Cardiovascular Health’ can provide the individual's heart health profile. Based on the individual's heart health profile, nutrients and supporting supplements that improve heart conditions can be incorporated into the blend and can be supplemented in the appropriate amount. Table 10 provides an exemplary list of biomarkers that can be tested in the ‘Cardiovascular Health’ test along with the respective nutrients, exemplary supporting supplements, exemplary additional supplements, and dosages for men and women that can improve the indicated biomarker levels and the associated heart condition.

In some embodiments, the nutritional supplement is administered to improve cardiovascular risks or functions. In some embodiments, the nutritional supplement comprises omega-3 fatty acids, coenzyme Q10, magnesium, or folic acid. In another embodiment, the nutritional supplement is administered to improve hypertension. In some embodiments, the nutritional supplement comprises L-arginine and L-citrulline.

In some embodiments, the cardiovascular biomarker is one or more biomarkers selected from Table 10. In some embodiments, the nutrient is one or more nutrients selected from Table 10. In some embodiments, the supporting supplement is one or more supporting supplements selected from Table 10. In some embodiments, the additional supplement is one or more additional supplements selected from Table 10. In some embodiments, the dosage for men or the dosage for women is the dosage for men or the dosage for women selected from Table 10.

### Neurological Condition

Neurological conditions such as mild cognitive impairment (MCI) and dementia can commonly occur in the geriatric population. Poor cognitive ability, stemming from various causes is also commonly observed. In some aspects, the method provided herein further comprises a further serum test that assesses neural health. The ‘Neural Health’ test assesses a strong set of neurological biomarkers and can help in the early detection of various neurological conditions. A ‘Neural Health’ test is commercially available from a variety of vendors including, but not limited to, Vibrant Wellness (test code VAREQUISTION116).

The results from the ‘Neural Health’ test can determine an individual's neurological status and help determine the appropriate supplementation to be provided. Table 11 provides an exemplary list of biomarkers that can be tested in the ‘Neural Health’ test along with exemplary respective nutrients, any exemplary supporting supplements, and dosages for men and women that can improve the biomarker levels and its associated condition.

In another embodiment, the nutritional supplement is administered to improve mild cognitive impairment. In some embodiments, the nutritional supplement comprises folate, vitamin E, omega-3 fatty acids and/or the supporting supplements, quercetin and curcumin.

In some embodiments, the neural biomarker is one or more biomarkers selected from Table 11. In some embodiments, the nutrient is one or more nutrients selected from Table 11. In some embodiments, the supporting supplement is one or more supporting supplements selected from Table 11. In some embodiments, the dosage for men or the dosage for women is the dosage for men or the dosage for women selected from Table 11.

### Thyroid Disease

Thyroid disease refers to the conditions associated with the thyroid gland, which produces thyroid hormone. Thyroid hormone is associated with various biological activities including metabolism. Women are more likely to suffer from thyroid disease than men. As a result, thyroid disease is commonly prevalent among women and may affect a women's health in various ways. Thyroid disease can impact a women's menstrual cycle, ability to conceive, and pregnancy. Thus, thyroid disease can actively affect the health and well-being of the individual. In some aspects, the method provided herein further comprises a serum test that assesses thyroid health. A ‘Thyroid disease’ test is commercially available from a variety of vendors including, but not limited to, Vibrant Wellness (test code VAREQUISTION106).

The results from the ‘Thyroid Panel’ can indicate the status of thyroid function in the body and can enable the incorporation of nutrients that will help improve the biomarkers of thyroid function and thyroid health. Table 12 provides an exemplary list of biomarkers that can be tested in the ‘Thyroid Panel’ test along with exemplary respective nutrients, any exemplary supporting supplements, and dosages for men and women that can improve the biomarker levels and its associated condition.

In some embodiments, the nutritional supplement comprises iodine, selenium, or zinc.

In some embodiments, the Thyroid biomarker is one or more biomarkers selected from Table 12. In some embodiments, the nutrient is one or more nutrients selected from Table 12. In some embodiments, the dosage for men or the dosage for women is the dosage for men or the dosage for women selected from Table 12.

### Kidney Disease

Kidney or renal disease can refer to conditions associated with affected or reduced kidney function. Exemplary kidney diseases include, but are not limited to, acute kidney injury, kidney cysts, kidney stones, and kidney infections. In some aspects, the method provided herein further comprises a serum test that assesses kidney health. A ‘kidney disease’ test is commercially available from a variety of vendors including, but not limited to, Vibrant Wellness.

The results from the ‘Kidney Health’ can indicate the status of the individual's kidney function. Based on the results, exemplary appropriate supplements can be administered to the individual to improve the same. Table 13 provides an exemplary list of biomarkers that can be tested in the ‘Kidney Health’ test along with exemplary respective nutrients, exemplary supporting supplements, and other supplements, and dosages for men and women that can improve the biomarker levels and its associated condition.

In some embodiments, the nutritional supplement comprises vitamin D. In some embodiments, the nutritional supporting supplement comprises one or more of quercetin, ginger, or chitosan.

In some embodiments, the kidney biomarker is one or more biomarkers selected from Table 13. In some embodiments, the nutrient is one or more nutrients selected from Table 13. In some embodiments, the supporting supplement is one or more supporting supplements selected from Table 13. In some embodiments, the dosage for men or the dosage for women is the dosage for men or the dosage for women selected from Table 13.

### Liver Disease

The liver mainly participates in metabolism, energy storage, and detoxification. As a result, an alteration in its function can have serious health implications. Liver diseases such as, but not limited to, liver infections, non-alcoholic fatty liver disease (NAFLD), non-alcoholic fatty liver (NAFL), non-alcoholic steatohepatitis (NASH), and liver cirrhosis, are commonly prevalent liver conditions. In some aspects, the method provided herein further comprises a serum test that assesses liver function. In some embodiments, the test is a ‘Hepatic Function Panel’ that assesses liver function. A ‘Hepatic Function Panel” test is commercially available from a variety of vendors including, but not limited to, Vibrant Wellness.

The results from the ‘Hepatic Function Panel’ can indicate the status of the individual's liver health and function. Based on the results, exemplary appropriate supplements can be administered to the individual to improve the same. Table 14 provides an exemplary list of biomarkers that can be tested in the ‘Hepatic Function Panel’ test along with the respective exemplary nutrients, exemplary supporting supplements, and other exemplary supplements, and dosages from men and women that can improve the biomarker levels and its associated condition.

In some embodiments, the nutritional supplement comprises iron, zinc, copper, vitamin E, NAC, or vitamin C. In some embodiments, the nutritional supporting supplement comprises one or more of curcumin, quercetin, ginger, or glucoraphanin.

In some embodiments, the hepatic biomarker is one or more biomarkers selected from Table 14. In some embodiments, the nutrient is one or more nutrients selected from Table 14. In some embodiments, the supporting supplement is one or more supporting supplements selected from Table 14. In some embodiments, the dosage for men or the dosage for women is the dosage for men or the dosage for women selected from Table 14.

### Absorption Testing Blend

In some aspects, after the diagnosis of exemplary nutrient deficiencies, the methods provided herein can be used to optimize nutrient levels by creating personalized nutrient supplement blends. Such personalized blends can be customized to fulfil the individual's nutrient requirements. In some embodiments, individuals can be first supplemented with an ‘Absorption Testing Blend’ (ATB) comprising a multi-nutrient blend comprised of vitamins, minerals, amino acids and fatty acids in dosages close to the Recommended Dietary Allowance (RDA) values or the safe or generally advised values for nutrients (Table 15). The upper limit for each dosage can be the 80th percentile of the established upper tolerable limit (UTL) for the given nutrient (Table 15). This can be a uniform supplement blend which can be the ‘base blend’ given to individuals. This blend can be administered for 3-6 months, 3 months, 4 months, 5 months, or 6 months. The changes in one or more of an individuals' serum, WBC, and RBCs can be used to determine the nutrient absorption factor. Table 15 provides nutrient RDA, generally advised values & upper tolerable limit (UTL).

In some embodiments, the method further comprises obtaining or having obtained an absorption factor determined for the subject, the absorption factor reflecting subject's utilization of a plurality of nutrients.

In some embodiments, determining the personalized dosage further comprises determining the personalized dosage of the nutritional supplement for the subject according to the absorption factor.

In some embodiments, the absorption factor for the subject is determined by: comparing pre-supplementation blood nutrient values to post-supplementation blood nutrient values.

In some embodiments, comparing pre-supplementation blood nutrient values to post-supplementation blood nutrient values comprises determining a difference between the pre-supplementation and post-supplementation blood nutrient values.

In some embodiments, the pre-supplementation blood nutrient values are determined from a blood sample obtained from the subject prior to providing a supplement.

In some embodiments, the post-supplementation blood nutrient values are determined from a blood sample obtained from the subject subsequent to providing a supplement.

In some embodiments, the supplement comprises one or more of Vitamin A Palmitate, Beta Carotene, Thiamine Mononitrate, Riboflavin 5 Phosphate, Nicotinic Acid, Calcium Pantothenate, Pyridoxine HCl, Biotin, Cyanocobalamin, Ascorbic Acid, Cholecalciferol, d-Alpha Tocopheryl Succinate, Vitamin K1, Vitamin K2 as Menaquinone-7), Folinic Acid (Folate), Ubiquinone, L-Selenomethionine, Sea Salt, Potassium chloride, Calcium carbonate, Zinc Picolinate, Manganese Glycinate, Ferrous sulfate, Magnesium Citrate, Copper Bisglycinate Chelate, Chromium picolinate, Myo-inositol, Potassium Iodide, Molybdenum Glycinate Chelate, Dipotassium phosphate, L-Glutathione (reduced), Cyanocobalamin, Choline bitartrate, N-Acetyl-L-Cysteine (NAC), L-Asparagine, L-Glutamine, L-Serine, L-Arginine, L-Citrulline, L-Isoleucine, L-Valine, L-Leucine, L-carnitine, L-Phenylalanine, DHA, EPA, Arachidonic Acid, Conjugated Linoleic Acid, and Omega-3 DHA/EPA (High DHA) 3:1.

In some embodiments, the nutrient is one or more nutrients selected from Table 15. In some embodiments, the dosage for men or the dosage for women is the dosage for men or the dosage for women selected from Table 15.

In some embodiments, the method further comprises obtaining an RDA value for the subject, wherein the RDA value is determined according to the subject's age, gender, or pregnancy or lactation status.

In some embodiments, determining the personalized dosage further comprises determining the personalized dosage of the nutritional supplement for the subject according to the RDA value.

In some embodiments, the composition of the ATB can include the ‘generally suitable’ or the ‘default’ nutrient forms in dosages close to the RDA or generally advised values. These default nutrient forms are given in Table 16.

In some embodiments, the supplementing nutrient is one or more nutrients selected from Table 16. In some embodiments, the supplementing nutrient is one or more nutrients default forms selected from Table 16.

In some embodiments, customized blends (ATB) with non-default nutrient forms specific to a subject's need can be determined via genetic testing as described in Table 3 and via the PHQ. Similarly, in some embodiments, if nutrient deficiencies are associated with an altered gut microbiome, as assessed by conducting the ‘Gut Zoomer’ test, then the ‘Gut Microbiome correction’ can guide the addition of the appropriate probiotics or prebiotics to improve the gut microbiome balance. In some embodiments, since the ‘Gut Zoomer’ is a comprehensive test assessing not only gut commensals but also gut viruses, fungi, parasites, worms, and other gut inflammation and insufficiency markers, the appropriate supplements for altered levels of these gut entities or markers can also be incorporated into the blend.

In some embodiments, common comorbidities, including but not limited to, heart conditions, hypertension, mild cognitive impairment (neurological disorders), thyroid disease, liver disease, or kidney disease, can also be accounted for while creating the ATB. Suitable forms of nutrients can be utilized in the ATB based on any test or condition described herein.

In some embodiments, the higher value of the ‘calculated dosage’ (from the dosage formula) and the RDA or generally advised value (Table 15) can be used when determining the composition of a nutrient supplementation blend (such as an ATB) to address the nutritional deficiencies assessed via the nutrient testing method disclosed herein. In some embodiments, supporting supplements can be incorporated into the blend based on their starting dosages as given in Table 7.

### Vibrant Absorption Factor (VAF)

In some embodiments, extracellular and intracellular blood testing for nutrient values in one or more of serum, RBC, and WBCs post-nutritional supplementation, can be carried out after 3, 4, 5, or 6 months. Improvement in nutrition can be determined by comparing the nutrient values in serum, RBC, and WBCs post-nutritional supplementation to the nutrient values in serum, RBC, and WBCs pre-nutritional supplementation. Based on the improvement, the change in the nutrient levels can be attributed to the per milligram dosage of the supplemented nutrients. The assessed nutritional change is the “Vibrant Absorption factor (VAF (see Equation (1) below). The VAF can be used to determine the subject's utilization of the supplemented nutrients, as determined by the difference in extracellular and intracellular nutrient values before and after supplementation. Based on the subject's utility or absorption of the nutrients, the dosage amount of the nutrient needed to be supplemented or increased to effect change of the subject's nutrient values into the optimum nutrient zone can be assessed. In various embodiments, the VAF is determined by administering a pre-determined formulation, referred to herein as the Absorption Testing Blend. In some embodiments, such calculations can be used to determine the appropriate dosages of nutrients required in the Customized Blend (CB) in order to modulate (increase or decrease) an individual's nutrient values to the optimum zone, specific detail of which will be described in next section.

$$VAF = ( post - supplementation blood nutrient values - pre - supplementation blood nutrient values / per milligram dosage of the supplemented nutrients \qquad ( 1 )$$

In one embodiment, the nutritional supplement is administered according to a dosage formula based on at least one factor selected from the group consisting of VAF, age, gender, body metrics, or genetics, or any combination thereof.

In some embodiments, the method further comprises administering or having administered the personalized dosage of the nutritional supplement to the subject.

In some embodiments, the method is repeated at least one, two, three, four, five, six, seven, eight, nine, ten, or more times.

In some embodiments, the personalized dosage of the nutritional supplement is changed after the at least one, two, three, four, five, six, seven, eight, nine, ten, or more repeats.

### Approaches to Optimizing Nutrient Levels

In some embodiments, appropriate dosages of nutrients required in the Customized Blend (CB) can be optimized through various approaches disclosed herein. In a first exemplary approach, the ATB is used to determine the appropriate dosage for the CB, and thus the first exemplary approach is also referred to as the “ATB approach.” A few exemplary methods following this approach are further described hereinafter.

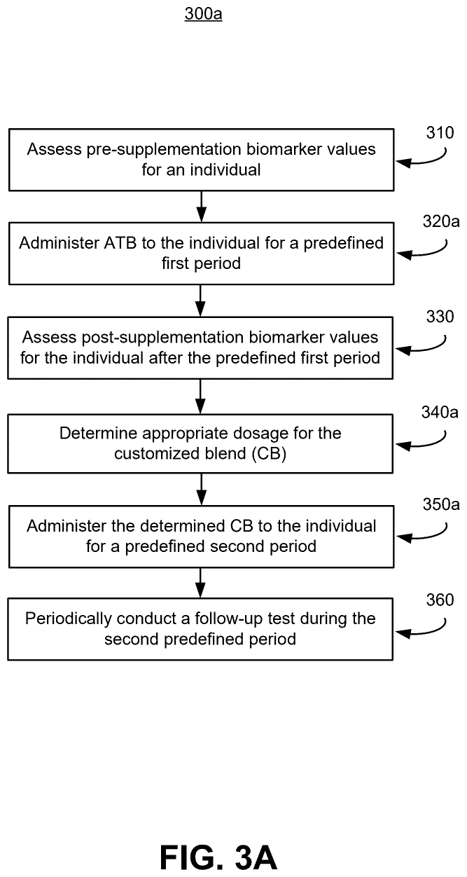

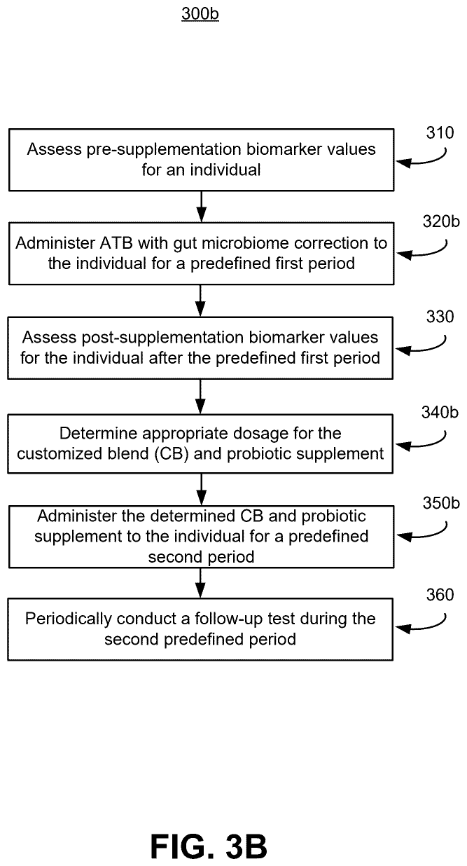

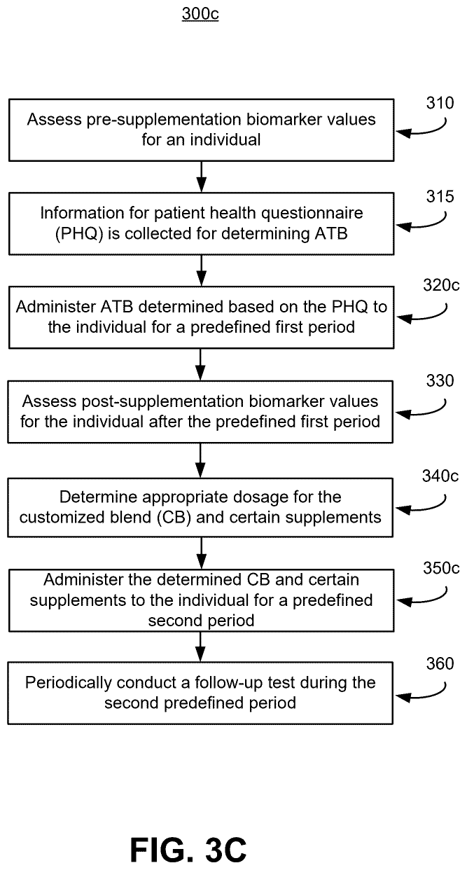

Referring to a first exemplary method 300a in FIG. 3A, initially, pre-supplementation biomarker values for an individual, including the individual's cellular and serum nutrient levels along with the genetic nutrient profile of the individual, are assessed at step 310. Next, the ATB is administered to the individual for a predefined period at step 320a, after which nutrient levels and the genetic nutrient profile are re-evaluated, to obtain the post-supplementation biomarker values at step 330. In some embodiments, the ATB is administered in powder, in capsule, or in other different formats.

In the next, the appropriate dosage for the CB is determined at step 340a. This includes determining the VAF based on the difference between post- and pre-supplementation biomarker values, which is divided by the ATB dosage. Subsequently, the TNV within the optimal nutrient range is established for each nutrient in both cellular and serum contexts, as described earlier in the “Nutrient metrics” section. The dosage of the CB for each nutrient is then determined based on the difference between the TNV and the post-test nutrient values, which is divided by the VAF (see Equation (2) below). After the determination of the CB for each nutrient, the individual is subject to the CB administration for a predefined period at step 350a.

$$Customized blend = ( TNV - Post test biomarker value ) / VAF \qquad ( 2 )$$

In some embodiments, a follow-up test is periodically conducted at step 360, to monitor and ensure improvement in nutrient levels after the CB administration. In some embodiments, based on the results from the follow-up tests, it is further determined whether the dosage of the CB for a nutrient requires adjustment. If the adjustment is necessary, appropriate modification is made to the CB, which is then administered to the individual again. In some embodiments, the personalized dosage of the nutritional supplement in the CB is modified as above after the at least one, two, three, four, five, six, seven, eight, nine, ten, or more repeats.

In some embodiments, various features disclosed elsewhere in the specification can be integrated into the above described method 300a.

Referring to FIG. 3B, in another exemplary method 300b, the gut microbiome correction is applied to the ATB when the ATB is administered to the individual. For example, at step 320b, the ATB with the gut microbiome correction is administrated to the individual instead of ATB. The specific type and amount of gut microbiome applied to the ATB may refer to the “Gut Microbiome Correction” section described earlier.

Still referring to FIG. 3B, in some embodiments, when calculating the appropriate dosage for the CB, the probiotic supplement is further added to the determined CB. For example, when calculating the appropriate dosage for the CB at step 340b, the probiotic supplement is also considered. Example probiotic supplements that can be added to the CB include but are not limited to Bifidobacterium such as Lactobacillus reuteri. At step 350b, the CB with the probiotic supplements is administered to the individual. In some embodiments, other steps in method 300b, including steps 310, 330, and 360, are similarly performed as described in method 300a.

Referring further to FIG. 3C, in another exemplary method 300c, the PHQ is performed at step 315, before administrating the ATB to the individual. Information obtained from the PHQ is used to select the best-suited form of nutrients for the individual. The best-suited forms are incorporated into the ATB before the ATB is administered to the individual at step 320c. The specific details regarding using PHQ to select the best-suited form of nutrients may refer to the “Questionnaire” section described earlier.

Still referring to FIG. 3C, in some embodiments, certain specific supplements and certain supporting supplements are added to the CB when determining the appropriate dosage for the CB at step 340c. Exemplary supplements include but are not limited to L-carnitine tartrate, and exemplary supporting supplements include but are not limited to quercetin and phosphatidylserine. At step 350c, the CB with the supplements is administered to the individual. In some embodiments, other steps in method 300c, including steps 310, 330, and 360, are similarly performed as described in method 300a.

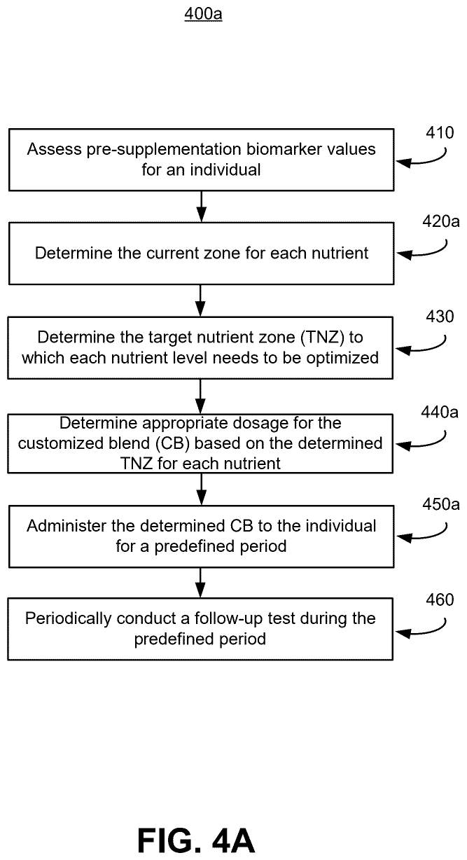

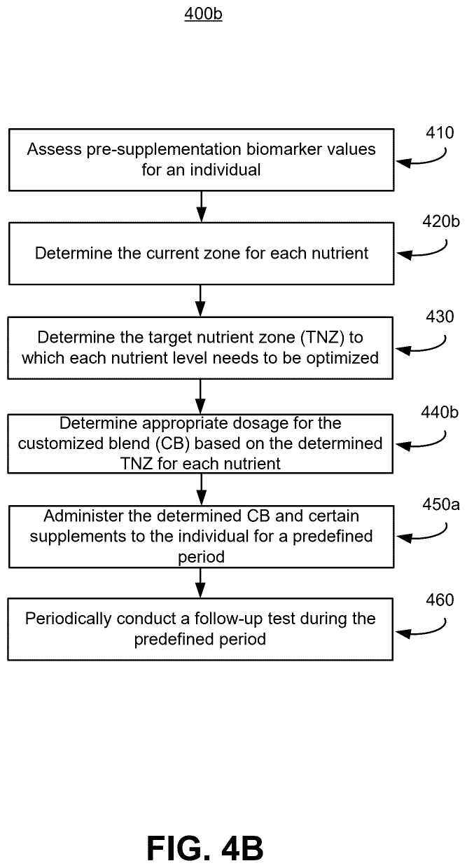

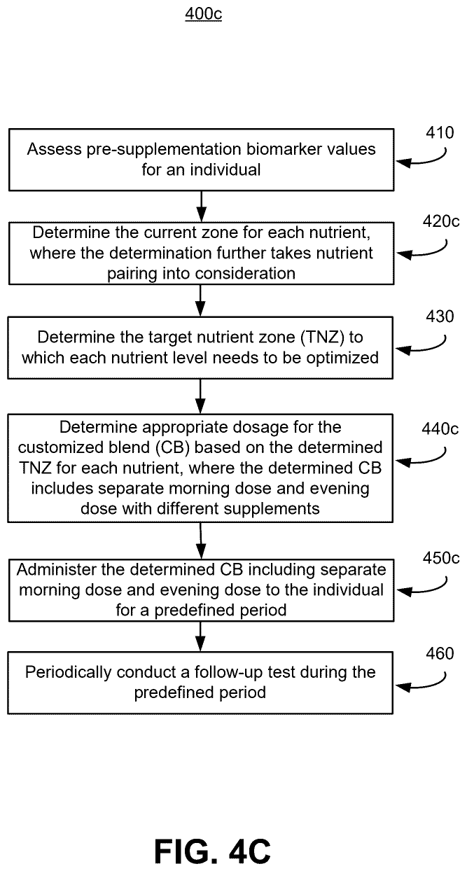

Referring now to FIGS. 4A-4C, a second exemplary approach to optimizing nutrient levels is further described. The second exemplary approach includes determining nutrient zones, and thus is referred to as the “Zone Approach.” According to some embodiments, the nutrient reference range for individuals can be divided into a number of zones, each indicating a nutrient wellness level. The number of zones may include any number of zones, for example, 2, 3, 4, 5, 6, 7, 8, 9, 10, or another number of zones. In one example, the number of zones includes five different zones, such as Zone 1, Zone 2, Zone 3, Zone 4, and Zone 5. Zone 1 represents deficiency, while Zones 4 and 5 represent optimal levels. In some embodiments, for a given nutrient, each zone is associated with a specific multiplication factor, which can be used to determine the appropriate dosage for the CB. A few exemplary methods following this Zone Approach are further described hereinafter.

Referring to a first exemplary method 400a in FIG. 4A, initially, one or more of an individual's cellular and serum nutrient levels, along with their nutrient genetic profile, is assessed at step 410. Based on the assessment results, the current zone for each nutrient is determined at step 420a. In some embodiments, the genetic predispositions and cellular levels may influence the determination of the overall zone for a nutrient. For example, if an individual's vitamin C level is in Zone 3 but the individual has a genetic predisposition for altered vitamin C uptake and poor cellular vitamin C level, the vitamin C level of the individual may be downgraded (e.g., to Zone 2 or to Zone 1 instead). This adjustment accounts for the impact of genetic polymorphisms and suboptimal cellular levels, each reducing the nutrient's zone by one or more zones. Therefore, this approach takes one or more of cellular and serum nutrient levels, along with their nutrient genetic profile into consideration to determine a given nutrient's overall zone.

In the next, the Target Nutrient Zone (TNZ) to which the individual's each nutrient level is to be optimized is determined at step 430. In one example, if a nutrient is already in the optimal zone, the nutrient does not need to be optimized. In some embodiments, the determined TNZ is used to determine the multiplication factor used to adjust the starting dosage at step 440a, resulting in the CB dosage for each nutrient. In various embodiments, the multiplication factor is a fixed constant value, previously determined for a nutrient when moving from a first zone to a second zone. For example, for a particular nutrient vitamin A, the multiplication factor when moving from Zone 1 to Zone 4 is a value of 3. As another example, for a particular nutrient vitamin B1, the multiplication factor when moving from Zone 1 to Zone 4 is a value of 3.5. The multiplication factor may differ for different nutrients, even when moving between the same zones (e.g., Zone 1 to Zone 4).

Generally, the multiplication factor is a larger value when moving between more interim zones as compared to when moving between fewer interim zones. For example, a multiplication factor for a particular nutrient may be a constant value of X when moving from Zone 1 to Zone 3, but the multiplication factor for the particular nutrient may be a larger constant value of Y when moving from Zone 1 to Zone 4 or when moving from Zone 1 to Zone 5. In some scenarios, a first multiplication factor for a first nutrient may be smaller than a second multiplication factor for a second nutrient factor, even when the first multiplication factor represents a movement between more interim zones. For example, a first multiplication factor for a first nutrient may be a constant value of X when moving from Zone 1 to Zone 4, but the multiplication factor for the second nutrient may be a larger constant value of Y when moving from Zone 1 to Zone 3. Thus, nutrients may have different, pre-determined multiplication values that are independent of other nutrients. Pre-determined multiplication values can be determined from analyzing data from multiple patients to determine a multiplication value for each nutrient.

The starting dosage (given in Table 6) for a given nutrient is multiplied by the TNZ-based multiplication factor, resulting in the CB dosage for each nutrient, as shown in Equation (3) below. At step 450a, the CB with the determined nutrient dosages is administered to the individual for a predefined period.

$$Customized Blend = ( Dosage * Multiplication Factor of the TNZ ) \qquad ( 3 )$$

In some embodiments, a follow-up test is periodically conducted at step 460, to monitor the progress towards the target nutrient levels during the predefined period, for example, to check whether a target zone is reached or not for a given nutrient. In some embodiments, based on the results from the follow-up tests, it is further determined whether the dosage of the CB for a nutrient requires adjustment. If the adjustment is necessary, appropriate modification is made to the CB, for example, by increasing the TNZ to increase the nutrient levels. The modified CB is administered to the individual, and a reassessment is conducted again. In some embodiments, the personalized dosage of the nutritional supplement in the CB is modified as above after the at least one, two, three, four, five, six, seven, eight, nine, ten, or more repeats.

In some embodiments, various features disclosed elsewhere in the specification can be integrated into the above described method 400a.

Referring to FIG. 4B, in another exemplary method 400b, the health comorbidities can be taken into consideration when determining the TNZ for a given nutrient. In one example, if the cardiovascular health of individual indicates a high LDL direct, the current zone for a given nutrient may be adjusted if necessary. Accordingly, in the exemplary method 300b, at step 420b for determining the current zone for each nutrient includes further adjusting the current zone for a given nutrient based on the health comorbidities.

Still referring to FIG. 4B, in some embodiments, certain specific supplements and certain supporting supplements are added to the CB when determining the appropriate dosage for the CB at step 440b. Exemplary supplements include but are not limited to Vitamin B2, and DHA, and exemplary supporting supplements include but are not limited to curcumin and broccoli. At step 450b, the CB with the supplements is administered to the individual.

In some embodiments, other steps in method 400b, including steps 410, 430, and 460, are similarly performed as described in method 400a.

Referring further to FIG. 4C, in another exemplary method 400c, nutrient paring is considered when determining the current zone for a nutrient at step 420c. As described elsewhere herein, nutrients can interact with each other to effectuate an overall impact on health. Several such interactions can be synergistic (positive) while some can be antagonistic (negative). For example, vitamin D helps the body to effectively absorb calcium, and both these nutrients together help in improving bone health. Accordingly, when determining the current zone for vitamin D, the current zone for calcium needs to be taken into consideration due to their synergistic effect.

Still referring to FIG. 4C, in some embodiments, certain supplements and/or certain supporting supplements are added to the CB when determining the appropriate dosage for the CB at step 440c. Exemplary supplements/supporting supplements include but are not limited to folate, vitamin B12, calcium, vitamin E, iron, and vitamin K. In some embodiments, not all of these supplements are administered in a same dose. Instead, these supplements may be separated by the time of intake. For example, in order to avoid competitive nutrient uptake, the CB may be separated by time of intake as morning dose and evening dose, with the competitive nutrients being supplemented in separate doses during the day. For example, folate, vitamin B12, calcium, and vitamin E can be included in the morning dose, while iron and vitamin K is included in the evening dose. At step 450c, the CB with the respective supplements is administered to the individual in separate morning dose and evening dose.

In some embodiments, other steps in method 400c, including steps 410, 430, and 460, are similarly performed as described in method 400a.

In some embodiments, in the above described methods, when determining the dosage of the CB, the CB dosage may not exceed the nutrient's UTL. For example, high dosages may be maintained at the 10th percentile below the nutrient's UTL to ensure safety.

In some embodiments, for nutrients tested at both cellular and serum levels, two TNV's or TNZ's (based on the specific approach, respectively) may lead to the generation of two CB dosages. In this case, the higher dosage may be selected as the final dosage for the CB. In some embodiments, for additional nutrient optimization based on the questionnaire responses or additional health tests, especially for those whose nutrient levels are not being tested, nutrients may be recommended based on RDA values.

In some embodiments, in the various optimization approaches described above, nutrient genetic polymorphisms will be addressed via the recommendation of the CB.

In some embodiments, additional approaches not described above may be used to optimize the nutrients. In one example, the CB is formulated by using proprietary blends. For example, a number of proprietary blends (e.g., 16 or another different number of blends) may be developed to address nutrient deficiencies. The number, composition, and purpose of these proprietary blends are subject to change under reasonable scope consistent with the descriptions in the disclosure. To optimize the nutrients of an individual, nutrients at one or more of the cellular, serum, and genetic levels are first assessed (e.g., by using NutriLite Test). Based on the test results, the CB may be created using the standard proprietary blends. The results will determine the combination and dosage of these blends that will be used to formulate the CB for the individual. In the next, the CB is administered to the individual for a specified period, with periodic serological assessments conducted to monitor and ensure improvement in nutrient levels. If nutrient levels are not optimized, the formulation and dosage of the proprietary blends can be adjusted to provide a new CB for the individual.

### Nutrient Pairing

Nutrients can interact with each other to effectuate an overall impact on health. Several such interactions can be synergistic (positive) while some can be antagonistic (negative). For example, vitamin D helps the body to effectively absorb calcium, and both these nutrients together help in improving bone health. Similarly, as an example, magnesium helps in the activation of vitamin D, and all of the enzymes that metabolize vitamin D require magnesium as a cofactor in the enzymatic reactions in the liver and kidneys. Thus, magnesium and vitamin D work well together to alter a subject's Vitamin D levels. Similarly, as an example, supplementing potassium when sodium is being supplemented is helpful, since potassium helps keep sodium levels in check avoiding the risk of hypertension. In some embodiments, nutrients can be co-supplemented to improve the effect of nutrient supplementation or to keep the nutrient balance in check.

In other cases, some nutrients can have antagonistic (negative) effects on each other. For example, intestinal absorption of iron and zinc occurs via the same protein, with zinc being predominantly absorbed. Supplementation of zinc and iron together will result in only zinc being taken up and not iron, which will not improve iron levels in the body. In such exemplary antagonistic cases, the blends can be separated by time of intake as morning dose and evening dose, with zinc and iron being supplemented in separate doses during the day. Also for example, high doses of vitamin E can counter-effect the action of vitamin K. Thus, considerations such as competitive uptake and the antagonistic effect of nutrients can be considered while creating the blend.

In some embodiments, a nutritional blend has a daily temporal component to administration. In some embodiments, antagonistic supplements are administered at different times. In some embodiments, antagonistic supplements are administered in the morning and the afternoon. In some embodiments, antagonistic supplements are administered in the morning and the evening. In some embodiments, antagonistic supplements are administered in the afternoon and the evening. Such times can be minutes to hours apart. In some embodiments, antagonistic supplements are administered 1, 2, 3, 4, 5, 6, 7, 8, 9, 10, 11, or 12 hours apart.

In some embodiments, the synergistic nutrient pairs can be supplemented together to improve the overall action of the paired nutrients. In some embodiments, the antagonistic nutrient pairs can be supplemented separately (e.g., separated by the time of intake as morning dose and evening dose).

Table 17 provides an exemplary list of synergistic nutrient pairings that can be included and antagonistic nutrient parings that can be avoided.

In some embodiments, the nutritional supplement comprises a combination of any of: calcium and vitamin D; magnesium and vitamin D; omega 3 and vitamin E; sodium and potassium; folate and vitamin B12; vitamin B3 and tryptophan; vitamin D and omega 3; and vitamin C and iron.

In some embodiments, the nutritional supplement does not include a combination of any of: iron and zinc; zinc and magnesium; copper and zinc; calcium and iron; and vitamin E and vitamin K.

### Dosage Formula

Disclosed herein are methods for providing nutritional supplements to subjects. In various embodiments, nutritional supplements are provided to subjects at personalized dosages to ensure that subjects are able to benefit from the nutritional supplements. For example, a customized blend of personalized dosages of nutrients within a nutritional supplement will fulfill the subject's individualized nutrient requirements. In various embodiments, the upper limit for a dosage will be the Nth percentile of the established UTL for the given nutrient. In various embodiments, the Nth percentile is one of the 20^{th} percentile, 30^{th} percentile, 40^{th} percentile, 50^{th} percentile, 60^{th} percentile, 70^{th} percentile, 80^{th} percentile, or 90^{th} percentile. In particular embodiments, the Nth percentile is the 80^{th} percentile.

In various embodiments, the personalized dosage of the nutritional supplement is determined for a subject according to various values, examples of which are described in further detail herein, e.g., in FIG. 2. For example, the personalized dosage of the nutritional supplement is determined for a subject according to one or more of extracellular (1) and intracellular (2) nutrient values, nutrient-related genetic predispositions (3), subject's body metrics (4), PHQ (5), appropriate supporting supplements to help ameliorate nutrient deficiency-associated conditions (6), gut microbiome correction (8), subject's VAF (9), and nutrient pairings (10). In various embodiments, the personalized dosage of the nutritional supplement is determined for a subject according to two or more, three or more, four or more, five or more, six or more, seven or more, eight or more, nine or more, or each of extracellular (1) and intracellular (2) nutrient values, nutrient-related genetic predispositions (3), subject's body metrics (4), PHQ (5), appropriate supporting supplements to help ameliorate nutrient deficiency-associated conditions (6), gut microbiome correction (7), health comorbidities (8), subject's VAF (9), and nutrient pairings (10).

In various embodiments, the personalized dosage of the nutritional supplement is determined for a subject according to extracellular nutrient values. For example, methods for providing a nutritional supplement for the subject can include obtaining levels of a plurality of extracellular nutrients measured from serum obtained from the subject. Thus, the personalized dosage of the nutritional supplement can be determined according to extracellular nutrients measured from serum obtained from the subject. Examples of extracellular nutrients are described herein (e.g., Table 2).

In various embodiments, the personalized dosage of the nutritional supplement is determined for a subject according to extracellular nutrient values and the subject's body metrics. An example of such a personalized dosage is shown below in Equation (4):

$$Dosage ( i ) = {( {TNV}_{( s e r u m )} / AN {V}_{( s e r u m )} )}_{( i )} * Body Metrics \qquad ( 4 )$$

where TNV represents the target nutrient value, ANV represents the actual nutrient value, and i represents the ith nutrient.

In various embodiments, the personalized dosage of the nutritional supplement is determined for a subject according to extracellular nutrient values, the subject's body metrics, and an RDA value. An example of such a personalized dosage is shown below in Equation (5):

$$Dosage ( i ) = {( {TNV}_{( s e r u m )} / AN {V}_{( s e r u m )} )}_{( i )} * Body Metrics * RD {A}_{( i )} \qquad ( 5 )$$

In various embodiments, the personalized dosage of the nutritional supplement is determined for a subject according to extracellular nutrient values and nutrient-related genetic predispositions. An example of such a personalized dosage is shown below in Equation (6) (where nutrient-related genetic predispositions is referred to as “polygenic factor” in Equation (6)):

$$Dosage ( i ) = polygenic {factor}_{( i )} * {( {TNV}_{( s e r u m )} / {ANV}_{( s e r u m )} )}_{( i )} \qquad ( 6 )$$

In various embodiments, the personalized dosage of the nutritional supplement is determined for a subject according to extracellular nutrient values, nutrient-related genetic predispositions, subject's body metrics, and an RDA value. An example of such a personalized dosage is shown below in Equation (7) (where nutrient-related genetic predispositions is referred to as “polygenic factor” in Equation (7)):

$$Dosage ( i ) = polygenic {factor}_{( i )} * {( {TNV}_{( s e r u m )} / {ANV}_{( s e r u m )} )}_{( i )} * Body Metrics * RD {A}_{( i} \qquad ( 6 )$$

In various embodiments, the personalized dosage of the nutritional supplement is determined for a subject according to intracellular nutrient values. For example, methods for providing a nutritional supplement for the subject can include obtaining levels of a plurality of intracellular nutrients from the subject. In various embodiments, the intracellular nutrients are measured from circulating blood cells. For example, the intracellular nutrients are measured from either red blood cells (RBCs) or white blood cells (WBCs). In such embodiments, the personalized dosage of the nutritional supplement can be determined according to intracellular nutrients measured from intracellular white blood cells (WBCs) or red blood cells (RBCs) obtained from the subject. Examples of intracellular nutrients are described herein (e.g., Table 2).

In various embodiments, the personalized dosage of the nutritional supplement is determined for a subject according to intracellular nutrient values and subject's body metrics. An example of such a personalized dosage is shown below in Equation (8):

$$Dosage ( i ) = {( {TNV}_{( cellular )} / {ANV}_{( cellular )} )}_{( i )} * Body Metrics \qquad ( 8 )$$

In various embodiments, the personalized dosage of the nutritional supplement is determined for a subject according to intracellular nutrient values, subject's body metrics, and RDA value. An example of such a personalized dosage is shown below in Equation (9):

$$Dosage ( i ) = {( {TNV}_{( cellular )} / {ANV}_{( cellular )} )}_{( i )} * Body Metrics * {RDA}_{( i )} \qquad ( 9 )$$

In various embodiments, the personalized dosage of the nutritional supplement is determined for a subject according to intracellular nutrient values and nutrient-related genetic predispositions. An example of such a personalized dosage is shown below in Equation (10) (where nutrient-related genetic predispositions is referred to as “polygenic factor” in Equation (10)):

$$Dosage ( i ) = polygenic {factor}_{( i )} * {( {TNV}_{( cellular )} / {ANV}_{( cellular )} )}_{( i )} \qquad ( 10 )$$

In various embodiments, the personalized dosage of the nutritional supplement is determined for a subject according to intracellular nutrient values, nutrient-related genetic predispositions, subject's body metrics, and an RDA value. An example of such a personalized dosage is shown below in Equation (11) (where nutrient-related genetic predispositions is referred to as “polygenic factor” in Equation (11)):

$$Dosage ( i ) = polygenic {factor}_{( i )} * {( {TNV}_{( cellular )} / {ANV}_{( cellular )} )}_{( i )} * Body Metrics * {RDA}_{( i} \qquad ( 11 )$$

In various embodiments, the personalized dosage of the nutritional supplement is determined for a subject according to both of extracellular (1) and intracellular (2) nutrient values. For example, methods for providing a nutritional supplement for the subject can include obtaining levels of a plurality of extracellular nutrients measured from serum obtained from the subject, and obtaining levels of a plurality of intracellular nutrients from the subject. In various embodiments, the intracellular nutrients are measured from circulating blood cells. For example, the intracellular nutrients are measured from either red blood cells (RBCs) or white blood cells (WBCs). In such embodiments, the personalized dosage of the nutritional supplement can be determined according to extracellular nutrients measured from serum obtained from the subject and intracellular nutrients measured from intracellular white blood cells (WBCs) or red blood cells (RBCs) obtained from the subject. Examples of extracellular and intracellular nutrients are described herein (e.g., Table 2).

In various embodiments, the personalized dosage of the nutritional supplement is determined for a subject according to extracellular nutrient values, intracellular nutrient values, and the subject's body metrics. An example of such a personalized dosage is shown below in Equation (12):

$${Dosage}_{( i )} = {( {TNV}_{( serum )} / {ANV}_{( serum )} )}_{( i )} * {( {TNV}_{( cellular )} / {ANV}_{( cellular )} )}_{( i ) )} * Body Metrics \qquad ( 12 )$$

In various embodiments, the personalized dosage of the nutritional supplement is determined for a subject according to extracellular nutrient values, intracellular nutrient values, the subject's body metrics, and an RDA value. An example of such a personalized dosage is shown below in Equation (13):

$${Dosage}_{( i )} = {( {TNV}_{( serum )} / {ANV}_{( serum )} )}_{( i )} * {( {TNV}_{( cellular )} / {ANV}_{( cellular )} )}_{( i ) )} * Body Metrics * {RDA}_{( i )} \qquad ( 13 )$$

In various embodiments, the personalized dosage of the nutritional supplement is determined for a subject according to extracellular nutrient values, intracellular nutrient values, and nutrient-related genetic predispositions. An example of such a personalized dosage is shown below in Equation (14) (where nutrient-related genetic predispositions is referred to as “polygenic factor” in Equation (14)):

$$Dosage ( i ) = polygenic {factor}_{( i )} * {( {TNV}_{( serum )} / {ANV}_{( serum )} )}_{( i )} * {( {TNV}_{( cellular )} / {ANV}_{( cellular )} )}_{( i )} \qquad ( 13 )$$

In various embodiments, the personalized dosage of the nutritional supplement is determined for a subject according to extracellular nutrient values, intracellular nutrient values, nutrient-related genetic predispositions, subject's body metrics, and an RDA value. An example of such a personalized dosage is shown below in Equation (15) (where nutrient-related genetic predispositions is referred to as “polygenic factor” in Equation (15)):

$$Dosage ( i ) = polygenic {factor}_{( i )} * {( {TNV}_{( serum )} / {ANV}_{( serum )} )}_{( i )} * {( {TNV}_{( cellular )} / {ANV}_{( cellular )} )}_{( i )} * Body Metrics * {RDA}_{( i )} \qquad ( 15 )$$

In various embodiments, the personalized dosage of the nutritional supplement is determined for a subject according to gut microbiome correction. In various embodiments, the gut microbiome correction value modifies the appropriate probiotics/prebiotics in dosage formula. Further details of the gut microbiome correction are described herein. In various embodiments, the the personalized dosage of the nutritional supplement is determined for a subject according to gut microbiome correction in addition to one or more of extracellular nutrient values, intracellular nutrient values, nutrient-related genetic predispositions, subject's body metrics, and an RDA value. An example of such a personalized dosage is shown below in Equation (16)

$$Dosage ( i ) = polygenic {factor}_{( i )} * {( {TNV}_{( serum )} / {ANV}_{( serum )} )}_{( i )} * {( {TNV}_{( cellular )} / {ANV}_{( cellular )} )}_{( i )} * {VAF}_{( i )} * Body Metrics * {RDA}_{( i )} * Gut Microbiome correction ( probiotics / prebiotics ) \qquad ( 16 )$$

In various embodiments, the personalized dosage of the nutritional supplement is determined for a subject according to an absorption factor (referred to herein as the “Vibrant Absorption Factor” or “VAF”). Further details of the VAF are described herein. In various embodiments, the the personalized dosage of the nutritional supplement is determined for a subject according to VAF in addition to one or more of extracellular nutrient values, intracellular nutrient values, nutrient-related genetic predispositions, subject's body metrics, and an RDA value. An example of such a personalized dosage is shown below in Equation (17):

$$Dosage ( i ) = polygenic {factor}_{( i )} * {( {TNV}_{( serum )} / {ANV}_{( serum )} )}_{( i )} * {( {TNV}_{( cellular )} / {ANV}_{( cellular )} )}_{( i )} * {VAF}_{( i )} * Body Metrics * {RDA}_{( i )} \qquad ( 17 )$$

In various embodiments, the personalized dosage of the nutritional supplement is determined for a subject according to one or more test-based health comorbidities, examples of which include cardiovascular health, neurological conditions, thyroid disease, kidney disease, and liver disease.

In various embodiments, the the personalized dosage of the nutritional supplement is determined for a subject according to one or more test-based health comorbidities in addition to one or more of extracellular nutrient values, intracellular nutrient values, nutrient-related genetic predispositions, subject's body metrics, and an RDA value. An example of such a personalized dosage involving a cardiovascular health comorbidity is shown below in Equation (18):

$$Dosage ( i ) = polygenic {factor}_{( i )} * {( {TNV}_{( serum )} / {ANV}_{( serum )} )}_{( i )} * {( {TNV}_{( cellular )} / {ANV}_{( cellular )} )}_{( i )} * {VAF}_{( i )} * Body Metrics * {RDA}_{( i )} * Cardiovascular Health - specific nutrients \qquad ( 18 )$$

Example cardiovascular health-specific nutrients include L-arginine and L-citrulline for hypertension.

Another example of such a personalized dosage involving a neurological health comorbidity is shown below in Equation (19):

$$Dosage ( i ) = polygenic {factor}_{( i )} * {( {TNV}_{( serum )} / {ANV}_{( serum )} )}_{( i )} * {( {TNV}_{( cellular )} / {ANV}_{( cellular )} )}_{( i )} * {VAF}_{( i )} * Body Metrics * {RDA}_{( i )} * Neurological Health - specific nutrients \qquad ( 19 )$$

Example neurological health-specific nutrients include folate, vitamin E, and omega-3 fatty acids and the supporting supplement, curcumin are supportive of neural health.

Another example of such a personalized dosage involving a thyroid health comorbidity is shown below in Equation (20):

$$Dosage ( i ) = polygenic {factor}_{( i )} * {( {TNV}_{( serum )} / {ANV}_{( serum )} )}_{( i )} * {( {TNV}_{( cellular )} / {ANV}_{( cellular )} )}_{( i )} * {VAF}_{( i )} * Body Metrics * {RDA}_{( i )} * Thyroid Health - specific nutrients \qquad ( 20 )$$

Example neurological health-specific nutrients include iodine, selenium, and zinc that aid in maintaining healthy thyroid function.

Another example of such a personalized dosage involving a kidney health comorbidity is shown below in Equation (21):

$$Dosage ( i ) = polygenic {factor}_{( i )} * {( {TNV}_{( serum )} / {ANV}_{( serum )} )}_{( i )} * {( {TNV}_{( cellular )} / {ANV}_{( cellular )} )}_{( i )} * {VAF}_{( i )} * Body Metrics * {RDA}_{( i )} * Kidney Health - specific nutrients \qquad ( 21 )$$

Example kidney health-specific nutrients include vitamin B6 and EPA, which help improve kidney stones.

Another example of such a personalized dosage involving a liver health comorbidity is shown below in Equation (22):

$$Dosage ( i ) = polygenic {factor}_{( i )} * {( {TNV}_{( serum )} / {ANV}_{( serum )} )}_{( i )} * {( {TNV}_{( cellular )} / {ANV}_{( cellular )} )}_{( i )} * {VAF}_{( i )} * Body Metrics * {RDA}_{( i )} * Kidney Health - specific nutrients \qquad ( 22 )$$

Example liver health-specific nutrients include zinc and supporting supplements such as licorice and ginger which boost liver functions.

### Compositions

The nutritional blends provided herein can be provided to the individual or subject as a powder, capsule, tablet, or emulsion. In some embodiments, the blend is provided to the individual or subject as a powder. In some embodiments, the blend is provided to the individual or subject as a capsule. In some embodiments, the blend is provided to the individual or subject as a tablet. In some embodiments, the blend is provided to the individual or subject as an emulsion. In some embodiments, the blend is provided to the individual or subject vi intravenous (IV) injection. The nutritional supplement blend can also be altered based on annual testing to customize nutrients according to the subject's changing nutritional needs.

In some embodiments, the nutritional supplement blend is modified based on genetic testing of the individual. In some embodiments, the nutritional supplement blend is modified based on the individuals experienced symptoms. In some embodiments, the nutritional supplement blend further comprises supporting supplements based on the conditions accompanied with nutrient deficiencies. In some embodiments, the nutritional supplement blend is customized based on age and gender as determined by the Recommended Dietary Allowance values. In some embodiments, the nutritional supplement blend is customized based on height and weight. In some embodiments, the nutritional supplement blend is customized based on alteration in gut microbiome. In some embodiments, the nutritional supplement blend is customized based on comorbidities via the assessment using the ‘additional health tests’ as disclosed herein. In some embodiments, the nutritional supplement blend is customized based on the time of intake to prevent competitive uptake of nutrients (e.g., a morning and an evening dose). In some embodiments, the nutritional supplement blend is customized based on the taste as per the individual's preference (e.g., multiple flavors such as grape, apple, watermelon, banana, cherry, raspberry, mango, etc).

In some embodiments, the nutritional supplement is provided as a powder, capsule, tablet, or emulsion, or any combination thereof.

In some embodiments, the nutritional supplement further comprises a flavouring agent.

In some embodiments, the supplements (e.g., either the ATB or the customized blend) can be provided in a powdered form. In such cases, the required intake per day for the individual can be indicated and the individual can scoop up the required amount using a spoon or similar other devices, dissolve it in an aqueous fluid (such as water, a shakes, a smoothie). The individual can then immediately consume it.

In one embodiment, the supplements can be supplemented in a capsule. The individual can orally consume the capsule as directed.

In one embodiment, the supplements can be supplemented in the form of a tablet which the individual can orally consume the tablet as directed.

In one embodiment, the supplements can be put together into an emulsion which can be ingested as a liquid drink. The emulsion can enable the dissolution of water- and fat-soluble nutrients.

In one embodiment, the supplements can be supplemented as a combination of at least two or more of a powder, a capsule, and a tablet.

In one embodiment, the supplements will be separated by the time of intake. In order to avoid competitive nutrient uptake, the blends will be separated by time of intake as morning dose and evening dose, with the competitive nutrients being supplemented in separate doses during the day. The intake of these separate doses will also be directed.

In one embodiment, the water-soluble supplements can together be supplemented as a flavourful blend while the insoluble components will be supplemented as capsules.

### Absorption Testing Blend (ATB)

In some aspects, provided herein are Absorption Testing Blends (ATB). An ATB can be a multi-nutrient supplement blend comprising the exemplary vitamins, minerals, amino acids, and fatty acids as described here. Exemplary dosages of the nutrients in the ATB can be similar to the RDA or generally advised values, as given in Table 15. In some embodiments, the dosages of the vitamins, minerals, amino acids, and fatty acids as described here in the ATB are within 0.25%, 0.5%, 0.75%, 1%, 2%, 3%, 4%, 5%, 6%, 7%, 8%, 9%, or 10%, or between about 0.25-10% of the RDA or generally advised values as described herein.

In one embodiment, the ATB comprises the ‘general’ or ‘default form’ of nutrients including vitamins, minerals, amino acids, and fatty acids in dosages close to the RDA or generally advised values, as given in Table 15.

In one embodiment, the ATB comprises the ‘suitable form’ of nutrients including vitamins, minerals, amino acids, and fatty acids, as determined from the genetic testing results and the PHQ, in dosages close to the RDA or generally advised values, as given in Table 15.

In another embodiment, the ATB comprises nutrients including vitamins, minerals, amino acids, and fatty acids in the calculated dosages for nutrients as required to improve the individual's nutrient status. In another embodiment, the ‘customized blend” comprises an altered ATB according to a specific individual.

### Customized Blend

In some aspects, provided herein are ‘customized blends’ comprising nutrients in dosages calculated based on the amount required optimize an individual's nutrient values (e.g., bring the individual's nutrient levels into the optimum nutrient zone), as determined by the ‘Dosage’ formula. Thus, based on the deficient nutrients as measured, the ‘customized blend” dosage can take into account the RDA or generally advised values along with additional nutrient values required to fulfil the individual's nutrient needs.

### EXAMPLES

Below are examples of specific embodiments for carrying out the present invention. The examples are offered for illustrative purposes only, and are not intended to limit the scope of the present invention in any way. Efforts have been made to ensure accuracy with respect to numbers used (e.g., amounts, temperatures, etc.), but some experimental error and deviation should, of course, be allowed.

The practice of the present invention will employ, unless otherwise indicated, conventional methods of protein chemistry, biochemistry, recombinant DNA techniques and pharmacology, within the skill of the art. Such techniques are explained fully in the literature. See, e.g., T. E. Creighton, Proteins: Structures and Molecular Properties (W.H. Freeman and Company, 1993); A. L. Lehninger, Biochemistry (Worth Publishers, Inc., current addition); Sambrook, et al., Molecular Cloning: A Laboratory Manual (2nd Edition, 1989); Methods In Enzymology (S. Colowick and N. Kaplan eds., Academic Press, Inc.); Remington's Pharmaceutical Sciences, 18th Edition (Easton, Pennsylvania: Mack Publishing Company, 1990); Carey and Sundberg Advanced Organic Chemistry 3^{rd} Ed. (Plenum Press) Vols A and B(1992).

### Example 1: NutriPro Test

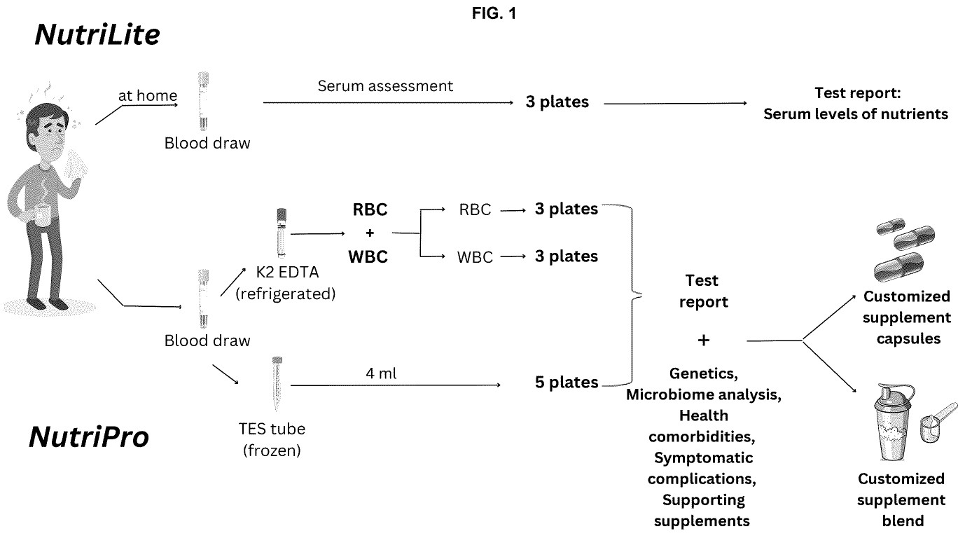

The ‘NutriPro’ test is used to assess serum (extracellular), RBC, and WBC (intracellular) nutrient values along with genetic testing for any predispositions with could be affecting nutrient values in the body. If required, additional tests such as the gut zoomer, cardiovascular health, thyroid test, kidney function test, liver function test, etc, are carried out. FIG. 1 provides a schematic diagram of the process of the NutriPro test from the individual subject to the creation of customized nutritional blends.

The subject individual fills out the health questionnaire (PHQ) which helps establishes factors such as symptoms, complications, comorbidities, and ongoing medications. The subject's blood is drawn and the extracellular serum and intracellular blood cells are tested for nutritional deficiencies via mass spectroscopy (e.g., Liquid chromatography-mass spectrometry (LC-MS), Gas chromatography-mass spectrometry (GC-MS), or Inductively coupled plasma-mass-spectrometry (ICP-MS)). FIG. 1 provides a diagram of the NutriPro test steps.

The NutriPro test results indicate any altered levels of nutrients in the subject. An ATB consisting of the default nutrient forms in dosages close to the RDA values or generally advised values for nutrients will be supplemented to the individual.

In addition, the subject's blood is subjected to genetic testing. Information obtained from genetic testing and the PHQ is used to select the best-suited form of nutrients for the individual. The best-suited forms are incorporated into the ATB if the individual chooses to customize the ATB. Similarly, supporting supplements to ameliorate nutrient deficiency-associated conditions are incorporated into the ATB. If the deficiency is associated with an alteration in the gut microbiome, then probiotics or prebiotics are also added to the blend to replenish the microbial species, as determined by the gut microbiome correction. Lastly, the presence of comorbidities such as heart conditions, hypertension, mild cognitive impairment, thyroid disease, etc. are taken into consideration, for those who request personalization of the ATB.

The ATB is given to the individual for nutritional supplementation, and blood testing for nutrients in serum, RBC, and WBC is carried out after 3 or 6 months.

Based on the improvement (in comparison with values prior to supplementation), the change in the nutrient levels are attributed to the per milligram dosage of the supplemented nutrients. The assessed change is called the VAF. The VAF is used to understand how the individual's body is utilizing these nutrients, depending on the difference in extracellular and intracellular nutrient values before and after supplementation. Based on the body's utility or absorption of the nutrients, the amount of nutrients needed to be supplemented to alter the individual's nutrient values into the optimum nutrient zone is understood. The VAF calculations help in determining the appropriate dosages of nutrients required in the ‘customized blend.’

The ‘Dosage’ formula is used to calculate the customized dosage to be used in the ‘customized blend.’ The supplement dosage is individualized for each individual and is determined using the ‘Dosage’ formula that takes into account factors like age, polygenic factor, target nutrient value (TNV), actual nutrient value (ANV) of the individual for both extracellular (serum) and intracellular (WBC and RBC) nutrient values, calculated VAF, and body metrics (weight, height) and RDA (based on gender and age).

After testing, the individual takes the ATB or the customized blend for the amount of time indicated. An improvement in the individual's nutritional health is observed. An improvement in the individual's comorbidities and overall health is also observed.

While the invention has been particularly shown and described with reference to a preferred embodiment and various alternate embodiments, it will be understood by persons skilled in the relevant art that various changes in form and details can be made therein without departing from the spirit and scope of the invention.

### Examples 2-3: ATB Approach to Optimizing Nutrient Levels

Below are examples demonstrating how nutrient levels can be optimized using the ATB approach described above. The tables provide the selected ATB dosages for each nutrient. While the ATB dosages may vary, the underlying formula used to achieve the CB remained consistent.

### Example 2: 18-Year-Old Male

An 18-year-old male athlete's serological profile indicated that he was deficient in several vitamins, minerals and amino acids. Cellular levels of the essential fatty acid, eicosapentaenoic acid (EPA), were also low. A genetic polymorphism in the folate gene, which affected the conversion of folate to its bioactive form, methyl folate, was leading to low circulating folate levels in the body. Taking his cellular nutrient levels and nutrient genetic profile into consideration, his cellular vitamin C and zinc levels were low due to poor cellular absorption.

The ATB was administered for 1 month after which nutrient values were retested. The VAF was calculated by establishing the difference between his pre- and post-supplementation nutrient values divided by the ATB. Following this, the TNV was determined which gave the CB by calculating the difference between the TNV and the post-supplementation biomarker values divided by the VAF. Additionally, the questionnaire established that the individual would like to improve his muscle soreness, as he is an athlete. Therefore, although not deficient, arginine was supplemented to improve its levels to the optimum range, which could support muscle health and recovery. The CB was taken for three months, after which nutrient levels were again retested. The follow-up tests indicated that the CB was able to improve the individual's nutrient levels, with their levels crossing the TNV into the optimum range.

Table 18 provides an exemplary optimization of an 18-year-old male's nutrient profile via the ATB approach.

### Example 3: 50-Year-Old Female

This 50-year-old female is menopausal. Her serological profile indicated deficiencies in several vitamins, minerals, and amino acids. Her cellular levels of manganese and docosahexaenoic acid (DHA) were also low. Genetic assessment revealed a polymorphism that leads to poor conversion of folate to its active form, L-methylfolate, which could have contributed to low circulating folate levels. Similarly, a polymorphism affecting selenium's transport resulted in deficient folate levels in the serum. Poor cellular uptake of vitamin C led to inadequate cellular vitamin C levels. Considering her serum and cellular nutrient levels, as well as her nutrient genetic profile, a genetic polymorphism causing poor conversion of vitamin A to its active form, retinol, resulted in low circulating and cellular levels of vitamin A. Additionally, a polymorphism affecting vitamin D3 transport led to low serum and cellular levels of vitamin D3.

The ATB was administered for 3 months after which nutrient values were retested. The VAF was calculated by establishing the difference between her pre- and post-supplementation nutrient values divided by the ATB. Following this, the TNV was determined which gave the CB by calculating the difference between the TNV and the post-supplementation biomarker values divided by the VAF. Additional health tests revealed that she was suffering from hypothyroidism. Therefore, although not deficient, zinc was supplemented to elevate zinc levels to the optimum range to support thyroid health. Furthermore, using RDA values, iodine was also supplemented to enhance thyroid function. Selenium, which supports thyroid function, was already being optimized due to existing deficiencies. The CB was taken for three months, after which nutrient levels were retested. The follow-up tests indicated that the CB successfully improved the individual's nutrient levels, with these levels crossing the TNV into the optimum range.

Table 19 provides an exemplary optimization of a 50-year-old female's nutrient profile via the ATB approach.

### Examples 4-5: Zone Approach to Optimizing Nutrient Levels

Below are examples demonstrating how nutrient levels can be optimized using the Zone Approach. The tables provide the selected starting dosages for each nutrient. While the starting dosages and the multiplication factor may vary, the underlying formula used to achieve the CB remained consistent.

### Example 4: 35-Year-Old Male

The 35-year-old male is a bodybuilder. His serological profile indicated deficiencies in several vitamins, minerals, and amino acids. All of these nutrients were falling under Zone 1 due to their levels. Although his cellular levels of the fatty acids, linoleic acid (LA) and docosapentaenoic acid (DPA), were falling under Zone 2, they were worked on to improve their levels to the optimum zone. Despite being in Zone 2, vitamin B1 was downgraded to Zone 1 due to its low cellular levels caused by poor cellular uptake from a genetic polymorphism in the concerned gene. Similarly, selenium had been declared to be in Zone 1 due to affected transport caused by a polymorphism.

On identifying the zones, the TNZ was determined, which gave the multiplication factor by which the starting dosage was to be multiplied to reach optimum nutrient levels. This gave the dosage of the CB for each nutrient, which was followed for three months. Additionally, the questionnaire established that the individual would like to improve his muscle mass as he is a bodybuilder. Therefore, carnitine was supplemented to improve its levels to the TNZ, which could support muscle health and mass. Follow-up testing indicated that most nutrient levels reached their TNZs, except for vitamin B1 which was supposed to reach Zone 4, and DPA and LA, which were supposed to reach the higher Zone 5, respectively. The CB dosage will be continued for several months until nutrients are optimized to the TNZ.

Table 20 provides an exemplary optimization of a 35-year-old male's nutrient profile via the Zone Approach.

### Example 5: 70-Year-Old Female

The 70-year-old female's serological profile indicated deficiencies in most vitamins, minerals, and amino acids. Cellular vitamin B1, EPA, and DHA were also low. All of these nutrients were categorized as Zone 1 due to their low levels, except L-glutamine which was in zone 2. Although not in a deficient zone, L-glutamine levels were optimized to reach the optimum zone.

Upon identifying the zones, the TNZ was determined, which provided the multiplication factor to adjust the starting dosage to reach optimal nutrient levels. This calculation gave the dosage of the CB for each nutrient, which was followed for three months. Follow-up testing indicated that only vitamin B1 and vitamin B6 moderately increased and managed to reach Zone 2. L-glutamine levels saw a marginal increase but remained in Zone 2. The remaining nutrients did not show substantial improvements, as they were still in Zone 1. The CB dosage is recommended for a period until nutrient zones have been optimized, and updates to the CB is considered if no improvement in nutrient levels is observed.

Table 21 provides an exemplary optimization of a 70-year-old female's nutrient profile via the Zone Approach.

All references, issued patents and patent applications cited within the body of the instant specification are hereby incorporated by reference in their entirety, for all purposes.

TABLE 1

| | | Analysis | | | |
|---|---|---|---|---|---|
| | Analyte | Serum | RBC | WBC | Genetics |
| Vitamins | Vitamin A (Retinol) | Yes | | Yes | rs12934922, |
| and | | | | | rs1667255, |
| minerals | | | | | rs6564851, |
| | | | | | rs7501331, |
| | | | | | rs11645428 |
| | Vitamin A (beta-carotene) | Yes | | | rs11645428 |
| | Vitamin B1 (Thiamine diphosphate) | Yes | Yes | Yes | rs17514104 |
| | Vitamin B2 (Riboflavin 5-Phosphate) | Yes | Yes | Yes | rs1799983 |
| | Vitamin B3 (Nicotinic acid) | Yes | Yes | Yes | |
| | Vitamin B5 (Pantothenic acid) | Yes | | Yes | |
| | Vitamin B6, Pyridoxal 5-Phosphate | Yes | Yes | Yes | |
| | Vitamin B7 (Biotin) | Yes | | | rs13078881 |
| | Vitamin B12 (Cyanocobalamin) | Yes | Yes | Yes | rs492602, |
| | | | | | rs526934, |
| | | | | | rs602662 |
| | Vitamin C (L-Ascorbic acid) | Yes | Yes | Yes | rs33972313, |
| | | | | | rs4257763, |
| | | | | | rs6139591, |
| | | | | | rs6596473 |
| | Vitamin D, 25-OH | Yes | | | rs10741657, |
| | | | | | rs12785878, |
| | | | | | rs2282679, |
| | | | | | rs10766197 |
| | Vitamin D3 (Cholecalciferol) | Yes | | Yes | rs10877012 |
| | Vitamin D, 1-25 dihydroxy | Yes | | | rs4588 |
| | Vitamin E (alpha-tocopherol) | Yes | Yes | Yes | rs12272004 |
| | Vitamin K1 (Phylloquinone) | Yes | | Yes | rs2108622 |
| | Vitamin K2 (Menaquinone-MK-7) | Yes | | Yes | |
| | Folate (L-5-methyltetrahydrofolate) | Yes | Yes | | rs1801131, |
| | | | | | rs1801133 |
| | Coenzyme Q10 (Ubiquinone + | Yes | Yes | Yes | rs775607037, |
| | Ubiquinol), Total | | | | rs786204770 |
| | Selenium, Se 76 | Yes | Yes | Yes | rs1050450, |
| | | | | | rs3877899 |
| | Sodium | Yes | Yes | | rs2304478, |
| | | | | | rs7204044 |
| | Potassium | Yes | Yes | | rs889299 |
| | Calcium, Ca 44 | Yes | Yes | Yes | rs4516035 |
| | Zinc, Zn 67 | Yes | Yes | Yes | rs11126936 |
| | Manganese, Mn 55 | Yes | Yes | Yes | rs13107325 |
| | Iron, Fe 56 | Yes | Yes | | rs1799945, |
| | | | | | rs1800562, |
| | | | | | rs3811647, |
| | | | | | rs4820268, |
| | | | | | rs855791 |
| | Magnesium, Mg 24 | Yes | Yes | | rs4680 |
| | Copper, Cu 63 | Yes | Yes | Yes | rs76151636 |
| | Chromium, Cr 53 | Yes | Yes | | |
| | Myo-Inositol | Yes | | | |
| | Iodine | Yes | Yes | | rs225014 |
| | Molybdenum | Yes | Yes | | rs594445 |
| | Phosphorus | Yes | Yes | | rs4074995 |
| | Tetrahydrobiopterin | Yes | Yes | | rs5030853, |
| | | | | | rs8007267 |
| | Fluoride | Yes | | | rs4284505 |
| | Copper/Zinc | Yes | | | |
| Amino | Glutathione Oxidized | Yes | Yes | Yes | rs121909307, |
| Acids | | | | | rs1695 |
| | MMA (Methylmalonic acid) | Yes | | | rs291466, |
| | | | | | rs121918252 |
| | Choline | Yes | | Yes | rs3733890, |
| | | | | | rs7946 |
| | L-Cysteine | Yes | | Yes | |
| | L-Asparagine | Yes | | Yes | |
| | L-Glutamine | Yes | | Yes | |
| | L-Serine | Yes | | Yes | |
| | L-Arginine | Yes | | | |
| | L-Citrulline | Yes | | | |
| | L-Isoleucine | Yes | | | |
| | L-Valine | Yes | | | |
| | L-Leucine | Yes | | | |
| | Free Carnitine | Yes | | Yes | |
| | Phenylalanine | | | | rs5030853 |
| Fatty | DHA (Docosahexaenoic acid) | Yes | Yes | | |
| Acids | EPA (Eicosapentaenoic acid) | Yes | Yes | | |
| | DPA (Docosapentaenoic acid) | Yes | Yes | | |
| | AA (Arachidonic acid) | Yes | Yes | | |
| | LA (Linoleic acid) | Yes | Yes | | rs174547 |
| | Omega-3 Total | | Yes | | |
| | Omega-6 Total | | Yes | | |
| | Omega-3 Index | | Yes | | |
| | AA/EPA | | Yes | | |

TABLE 2

| | | Serum | | RBC | | WBC | |
|---|---|---|---|---|---|---|---|
| | | | 90^{th} | | 90^{th} | | 90^{th} |
| | | | percentile | | percentile | | percentile |
| | | | of the | | of the | | of the |
| | | Nutrient | nutrient | Nutrient | nutrient | Nutrient | nutrient |
| | | reference | reference | reference | reference | reference | reference |
| | Nutrient | range | range | range | range | range | range |
| Vitamins and | Vitamin A (Retinol) | 116.48-135.71 | 133.787 | | | 0.9-17.3 | 16.5 |
| minerals | | (mcg/dL) | (mcg/dL) | | | (pg/MM WBC) | (pg/MM WBC) |
| | Vitamin A (beta-carotene) | 50-300 | 275 | | | | |
| | | (mcg/dL) | (mcg/dL) | | | | |
| | Vitamin B1 (Thiamine | 72.28-107.31 | 103.80 | | | 0.1-7.0 | 6.8 |
| | diphosphate) | (nmol/L) | (nmol/L) | | | (pg/MM WBC) | (pg/MM WBC) |
| | Vitamin B2 (Riboflavin 5- | 177.78-261.71 | 253.317 | | | 0.2-3.6 | 3.4 |
| | Phosphate) | (mcg/L) | (mcg/L) | | | (pg/MM WBC) | (pg/MM WBC) |
| | Vitamin B3 (Nicotinic acid) | 52.08-76.81 | 74.337 | | | 39.6-303.5 | 300.5 |
| | | (ng/mL) | (ng/mL) | | | (pg/MM WBC) | (pg/MM WBC) |
| | Vitamin B5 (Pantothenic | 237.9-346.5 | 335.64 | | | 2.5-32.8 | 30.2 |
| | acid) | (mcg/L) | (mcg/L) | | | (pg/MM WBC) | (pg/MM WBC) |
| | Vitamin B6, Pyridoxal 5- | 298.64-447.6 | 432.704 | | | 0.5-9.7 | 8.2 |
| | Phosphate | (ng/mL) | (ng/mL) | | | (pg/MM WBC) | (pg/MM WBC) |
| | Vitamin B7 (Biotin) | 133-329 | 309.4 | | | | |
| | | (pmol/L) | (pmol/L) | | | | |
| | Vitamin B12 | 907.4-1245.1 | 1211.63 | | | 2.00-11.99 | 10.85 |
| | (Cyanocobalamin) | (ng/L) | (ng/L) | | | | |
| | Vitamin C (L-Ascorbic acid) | 1.62-2.22 | 2.16 | | | 0.5-9.7 | 9.2 |
| | | (mg/dL) | (mg/dL) | | | (ng/MM WBC) | (ng/MM WBC) |
| | Vitamin D, 25-OH | 42.68-49.01 | 48.877 | | | | |
| | | (ng/mL) | (ng/mL) | | | | |
| | Vitamin D3 (Cholecalciferol) | 6.78-9.51 | 9.237 | | | 25.9-246.6 | 243.1 |
| | | (ng/mL) | (ng/mL) | | | (pg/MM WBC) | (pg/MM WBC) |
| | Vitamin D, 1-25 dihydroxy | 25-80 | 74.5 | | | | |
| | | (ng/mL). | (ng/mL) | | | | |
| | Vitamin E (alpha-tocopherol) | 31.12-37.12 | 36.52 | | | 18.4-1031.1 | 1025.2 |
| | | (mg/L) | (mg/L) | | | (pg/MM WBC) | (pg/MM WBC) |
| | Vitamin K1 (Phylloquinone) | 8.94-13.3 | 12.864 | | | 0.10-0.71 | 0.68 |
| | | (ng/mL) | (ng/mL) | | | (pg/MM WBC) | (pg/MM WBC) |
| | Vitamin K2 (Menaquinone- | 0.0968-0.10648 | 0.105512 | | | 0.10-0.89 | 0.72 |
| | MK-7) | (ng/ml) | (ng/mL) | | | (pg/MM WBC) | (pg/MM WBC) |
| | Folate (L-5- | 14.24-16.6 | 16.5785 | ≥95.5 | | | |
| | methyltetrahydrofolate) | (ng/mL) | (ng/mL) | (ng/ml) | | | |
| | Coenzyme Q10 (Ubiquinone + | 1.578-2.071 | 2.0217 | | | 39.6-225.3 | 222.1 |
| | Ubiquinol), Total | (mcg/mL) | (mcg/mL) | | | (pg/MM WBC) | (pg/MM WBC) |
| | Selenium, Se 76 | 240.22-276.32 | 272.71 | | | 234-1050 | 1044 |
| | | (ng/mL) | (ng/mL) | | | (pg/MM WBC) | (pg/MM WBC) |
| | Sodium | 142.2-145.2 | 144.9 | | | | |
| | | (mmol/L) | (mmol/L) | | | | |
| | Potassium | 4.84-5.5 | 5.434 | | | | |
| | | (mmol/L) | (mmol/L) | | | | |
| | Calcium, Ca 44 | 10.04-10.6 | 10.544 | | | 15-120 | 118.2 |
| | | (mg/dL) | (mg/dL) | | | (ng/MM WBC) | (ng/MM WBC) |
| | Zinc, Zn 67 | 1.32-1.62 | 1.59 | | | 4-15 | 13.5 |
| | | (mcg/mL) | (mcg/mL) | | | (ng/MM WBC) | (ng/MM WBC) |
| | Manganese, Mn 55 | 1.814-2.2 | 2.1614 | | | 2-75 | 72.1 |
| | | (ng/mL) | (ng/mL) | | | (pg/MM WBC) | (pg/MM WBC) |
| | Iron, Fe 56 | 111.68-150.01 | 146.177 | 88.9-117.0 | 98.2 | | |
| | | (mcg/dL) | (mcg/dL) | (mg/dL) | (mg/dL) | | |
| | Magnesium, Mg 24 | 2.28-2.61 | 2.577 | 3.6-7.7 | 7.2 | | |
| | | (mg/dL) | (mg/dL) | (mg/dL) | (mg/dL) | | |
| | Copper, Cu 63 | 1.44-1.8 | 1.764 | | | 2-15 | 12.2 |
| | | (mcg/mL) | (mcg/mL) | | | (ng/MM WBC) | (ng/MM WBC) |
| | Chromium, Cr 53 | 0.242-0.302 | 0.296 | | | | |
| | | (ng/mL) | (ng/mL) | | | | |
| | Myo-Inositol | 40.52-50.62 | 49.61 | | | 0.10-2.50 | 2.12 |
| | | (nmol/mL) | (nmol/mL) | | | (ng/MM WBC) | (ng/MM WBC) |
| | Iodine | 40-92 | 86.8 | | | | |
| | | (mcg/L) | (mcg/L) | | | | |
| | Molybdenum | 0.28-1.17 | 1.081 | | | | |
| | | (ng/mL) | (ng/mL) | | | | |
| | Phosphorus | 2.8-4.5 | 4.33 | | | | |
| | | (mg/dL) | (mg/dl) | | | | |
| | Tetrahydrobiopterin | | | | | | |
| | Fluoride | 0.21-2.11 | 1.49 | | | | |
| | | (μmol/l) | (μmol/l) | | | | |
| | Copper/Zinc | 0.54-1.68 | 1.566 | | | | |
| Amino Acids | Glutathione Oxidized | 176-323 | 308.3 | | | 98.7-1163.0 | 1158.4 |
| | | (ug/ml) | (ug/ml) | | | (pg/MM WBC) | (pg/MM WBC) |
| | MMA (Methylmalonic acid) | 0.07-0.27 | 0.25 | | | | |
| | | (mmol/L) | (mmol/L) | | | | |
| | Choline | 18.94-24.1 | 23.584 | | | 0.2-1.5 | 1.28 |
| | | (nmol/mL) | (nmol/mL) | | | (ng/MM WBC) | (ng/MM WBC) |
| | L-Cysteine | 27.74-36.1 | 35.264 | | | 60.0-565.0 | 554.7 |
| | | (nmol/mL) | (nmol/mL) | | | (pg/MM WBC) | (pg/MM WBC) |
| | L-Asparagine | 100.48-131.81 | 128.677 | | | 0.5-2.8 | 2.25 |
| | | (nmol/mL) | (nmol/mL) | | | (ng/MM WBC) | (ng/MM WBC) |
| | L-Glutamine | 579.7-721.8 | 707.59 | | | 1.4-7.0 | 6.4 |
| | | (nmol/mL) | (nmol/mL) | | | (ng/MM WBC) | (ng/MM WBC) |
| | L-Serine | 112.42-139.62 | 136.9 | | | 1.8-19.8 | 18.1 |
| | | (nmol/mL) | (nmol/mL) | | | (ng/MM WBC) | (ng/MM WBC) |
| | L-Arginine | 200.1-249 | 244.11 | | | | |
| | L-Citrulline | 38.7-47.5 | 46.62 | | | | |
| | L-Isoleucine | 105.6-158.9 | 153.57 | | | | |
| | L-Valine | 256.1-368.0 | 356.81 | | | | |
| | L-Leucine | 180.1-249.3 | 242.38 | | | | |
| | Free Carnitine | 36.94-47.1 | 46.084 | | | | |
| | | (nmol/mL) | (nmol/mL) | | | | |
| | Phenylalanine | 1-10 | 9.1 | | | | |
| | | (mg/dL) | (mg/dL) | | | | |
| Fatty Acids | DHA (Docosahexaenoic acid) | | | 2.42-10.52 | 9.82% | | |
| | | | | (%) | | | |
| | EPA (Eicosapentaenoic acid) | | | 0.15-2.26 | 1.78% | | |
| | | | | (%) | | | |
| | DPA (Docosapentaenoic | | | 0.45-1.80 | 1.56% | | |
| | acid) | | | (%) | | | |
| | AA (Arachidonic acid) | | | 5.50-19.01 | 17.50% | | |
| | | | | (%) | | | |
| | LA (Linoleic acid) | | | 3.22-10.49 | 8.25% | | |
| | | | | (%) | | | |
| | Omega-3 Total | | | 3.25-13.99 | 11.25% | | |
| | | | | (%) | | | |
| | Omega-6 Total | | | 11.03-34.96 | 31.84% | | |
| | | | | (%) | | | |
| | Omega-3 Index | | | 8.00-12.65 | 10.24% | | |
| | | | | (%) | | | |
| | AA/EPA | | | 2.5-10.9 | 7.2 | | |

TABLE 3

| Polymorphism | | Polymorphism | | General | Alternate effective |
|---|---|---|---|---|---|
| Category | Nutrient | (gene) | Outcome | supplement | supplement |
| Conversion | Vitamin A | BCMO1 | Poor conversion to the active | Beta-carotene | Retinol |
| | (Retinol) | (rs12934922, rs6564851, | form of vitamin A | (Precursor form) | (Active form) |
| | | rs7501331, rs11645428) | | | |
| | Vitamin A | BCMO1 | Poor conversion of beta- | Beta-carotene | Retinol |
| | (Beta-Carotene) | (rs11645428) | carotene to retinol | (Precursor form) | (Active form) |
| | Vitamin D, 25 - OH | CYP2R1 | Poor vitamin D conversion to | Cholecalciferol | 25-hydroxyvitamin D |
| | | (rs10766197, | its hydroxylated form, 25- | (Precursor form) | (Hydroxylated form) |
| | | rs10741657 | hydroxyvitamin D | | |
| | | | (25(OH)D) | | |
| | Vitamin D3 | CYP27B1 | Poor conversion of vitamin D | Cholecalciferol | Calcitriol |
| | (Cholecalciferol) | (rs10877012) | to its active form, 1,25- | (Precursor form) | (Active form) |
| | | | dihydroxyvitamin (or | | |
| | | | calcitriol) | | |
| | Folate | MTHFR | Poor conversion of folate to | Folic acid | L-methylfolate |
| | (Vitamin B9) | (rs1801131, rs1801133) | its active form, L- | (Precursor form) | (Active form) |
| | | | methylfolate | | |
| | Choline | PEMT | Impaired conversion of | Supplementation | Phosphati-dylcholine |
| | | (rs7946) | phosphatidyl-ethanolamine | of choline | |
| | | | (PE) into phosphatidyl | | |
| | | | choline (PC) | | |
| | Linoleic Acid | FADS1 | Impaired conversion of | Linoleic acid | Linoleic acid |
| | | (rs174547) | linoleic acid leading to its | supplementation | supplementation is reduced |
| | | | accumulation | is reduced | |
| Absorption | Vitamin B1 | SLC35F3 | Impaired cellular uptake of | Vitamin B1 | Increased vitamin B1 |
| | (Thiamine | (rs17514104) | vitamin B1 | supplementation | supplementation to increase |
| | diphosphate) | | | | cellular uptake |
| | Vitamin B12 | FUT2, TCN1 | Impaired cellular uptake of | Vitamin B12 | Increased vitamin B12 |
| | (Cyanocobalamin) | (rs492602, rs602662, | vitamin B12 | supplementation | supplementation to increase |
| | | rs526934) | | | cellular uptake |
| | Vitamin C (L- | SLC23A1, SLC23A2 | Impaired cellular uptake of | Vitamin C | Increased vitamin C |
| | Ascorbic acid) | (rs33972313, rs4257763, | vitamin C | supplementation | supplementation to increase |
| | | rs6139591, rs6596473) | | | cellular uptake |
| | Sodium | SLC12A3 | Excess sodium reabsorption | Sodium | Sodium supplementation is |
| | | (rs2304478, rs7204044) | | supplementation | reduced |
| | | | | is reduced | |
| | Potassium | SCNN1B | Impaired absorption of | Potassium | Increased potassium |
| | | (rs889299) | potassium leading to loss of | supplementation | supplementation to make up |
| | | | potassium from the body | | for the loss of potassium |
| | | | | | from the body |
| | Calcium, Ca 44 | VDR | Affected intestinal calcium | Calcium | Increased calcium |
| | | (rs4516035) | absorption | supplementation | supplementation to increase |
| | | | | | cellular uptake |
| | Zinc, Zn 67 | SLC30A3 | Affected absorption of zinc | Zinc | Increased zinc |
| | | (rs11126936) | | supplementation | supplementation to increase |
| | | | | | cellular uptake |
| | Manganese, Mn 55 | SLC39A8 | Impaired absorption of | Manganese | Increased manganese |
| | | (rs13107325) | manganese | supplementation | supplementation to increase |
| | | | | | cellular uptake |
| | Iron, Fe 56 | HFE | Excess retention of iron in | Iron | Iron supplementation is |
| | | (rs1799945, rs1800562) | the body. | supplementation | reduced |
| | | | | is reduced | |
| | Copper, Cu 63 | ATP7B | Excess retention of copper in | Iron | Iron supplementation is |
| | | (rs76151636) | the body. | supplementation | reduced |
| | | | | is reduced | |
| | Phosphorus | RGS14 | Poor phosphorus re- | Phosphorus | Increased phosphorus |
| | | (rs4074995) | absorption | supplementation | supplementation to make up |
| | | | | | for the loss of phosphorus |
| Activity/ | Vitamin D, 25-OH | NADSYN1 | Affected activity of the gene | Vitamin D | Vitamin D supplementation |
| Function | | (rs12785878) | which is linked to serum | supplementation | |
| | | | vitamin D concentrations | | |
| | Vitamin B2 | NOS3 | Poor gene function elevates | Vitamin B2 | Increased supplementation |
| | (Riboflavin 5- | (rs1799983) | the risk of blood pressure | supplementation | of the cofactor, |
| | Phosphate) | | (BP) | | vitamin B2 which promotes |
| | | | | | NOS3 function along with |
| | | | | | L-arginine and L-citrulline |
| | | | | | supplementation which can |
| | | | | | lower high BP |
| | Vitamin B7 (Biotin) | BTD | Poor enzyme activity | Vitamin B7 | Increased vitamin B7 |
| | | (rs13078881) | involved in the reuse and | supplementation | supplementation which can |
| | | | recycling of vitamin B7 | | be readily utilized |
| | Vitamin K1 | CYP4F2 | Affected gene activity | Vitamin K | Vitamin K supplementation |
| | (Phylloquinone) | (rs2108622) | associated with vitamin K | supplementation | is reduced |
| | | | function which can affect the | is reduced | |
| | | | effectiveness of warfarin | | |
| | Selenium, Se 76 | GPX1 | Affected gene associated | Selenium | Supplementation of |
| | | (rs1050450) | with selenium status | supplementation | selenium along with other |
| | | | | | antioxidants to combat the |
| | | | | | oxidative stress associated |
| | | | | | with poor selenium status |
| | Magnesium, Mg 24 | COMT | Alteration in a gene that is | Magnesium | Supplementation of |
| | | (rs4680) | regulated/governed by | supplementation | magnesium along with other |
| | | | magnesium | | B vitamins (B2, B6, B9, and |
| | | | | | B12) that will enhance |
| | | | | | COMT activity |
| | Iodine | DIO2 | Affected iodine-associated | Iodine | Iodine supplementation |
| | | (rs225014) | gene which affects thyroid | supplementation | along with selenium and |
| | | | hormones | | zinc supplementation which |
| | | | | | will aid in maintaining |
| | | | | | healthy thyroid function |
| | Molybdenum | MOCOS | Affected molybdenum | Molybdenum | Increased molybdenum |
| | | (rs594445) | cofactor enzyme | supplementation | supplementation |
| | Fluoride | ESR1 | Affected enamel | Reduced fluoride | Reduced fluoride |
| | | (rs4284505) | mineralization in the | supplementation | supplementation; instead, |
| | | | presence of fluoride leading | | supplementation of calcium |
| | | | to fluorosis | | and magnesium which will |
| | | | | | reduce fluoride absorption |
| | Glutathione | GSTP1 | Affected modification of | Glutathione | Supplementation of |
| | Oxidized | (rs1695) | toxic compounds to | supplementation | glutathione along with N- |
| | | | glutathione | | acetyl cysteine which |
| | | | | | promotes glutathione levels |
| | Methylmalonic | HICBH | Variation in MMA levels | Vitamin B12 is | Vitamin B12 is |
| | acid (MMA) | (rs291466) | which can be associated with | supplemented | supplemented |
| | | | vitamin B12 deficiency | | |
| | | MUT | Affected activity of the | Vitamin B12 is | Vitamin B12 is |
| | | (rs121918252) | enzyme that metabolizes fat | supplemented | supplemented along with L- |
| | | | and protein leading to the | | carnitine which will |
| | | | accumulation of MMA. | | increase the elimination of |
| | | | | | MMA from the body |
| Transport | Vitamin D, 25 - OH | GC | Poor vitamin D transport | Vitamin D | Increased vitamin D |
| | | (rs2282679) | | supplementation | supplementation to increase |
| | | | | | the chances of being |
| | | | | | transported |
| | Vitamin E (alpha- | APOA5 | Affected vitamin E transport | Vitamin E | Increased vitamin D |
| | tocopherol) | (rs12272004) | | supplementation | supplementation to increase |
| | | | | | the chances of being |
| | | | | | transported |
| | Selenium, Se 76 | SEPP1 | Affected transport of | Selenium | Increased selenium |
| | | (rs3877899) | selenium | supplementation | supplementation to increase |
| | | | | | the chances of being |
| | | | | | transported |
| | Iron, Fe 56 | TF | Impaired iron transportation | Iron | Increased iron |
| | | | | supplementation | supplementation to increase |
| | | | | | the chances of being |
| | | | | | transported |
| Regulation | Vitamin D, 1-25 | VDR | Affected function of the | Vitamin D | Increased vitamin D |
| | dihydroxy | (rs4588) | factor that regulates vitamin | supplementation | supplementation along with |
| | | | D in the body | | calcium whose homeostasis |
| | | | | | is also affected due to the |
| | | | | | SNP |
| | Iron, Fe 56 | TMPRSS6 | Impaired iron content | Iron | Increased iron |
| | | (rs4820268, rs855791) | regulation due to low | supplementation | supplementation to increase |
| | | | hepcidin levels | | hepcidin levels |
| Metabolism | Coenzyme Q10 | COQ4 | Impaired production of | Coenzyme Q10 | Increased coenzyme Q10 |
| | (Ubiquinone + | (rs775607037, | coenzyme Q10 | supplementation | supplementation |
| | Ubiquinol), Total | rs786204770) | | | |
| | Tetrahydrobiopterin | PAH, GCH1 | Affected production of | Tetrahydro-biopterin | Increased tetrahydro- |
| | | (rs5030853, rs8007267) | tetrahydrobiopterin | supplementation | biopterin supplementation |
| | Glutathione | GSS | Impaired production of | Glutathione | Increased glutathione |
| | Oxidized | (rs121909307) | glutathione | supplementation | supplementation |
| | Choline | BHMT | Affected choline metabolism | Choline | Increased choline |
| | | (rs3733890) | | supplementation | supplementation |
| | Phenylalanine | PAH | Failure of phenylalanine | Phenylalanine | Phenylalanine |
| | | (rs5030853) | metabolism | supplementation | supplementation is reduced |
| | | | | is reduced | |

TABLE 4

| | | Genotype score | | | |
|---|---|---|---|---|---|
| Nutrient | rs ID | Wild | Het | Mutant | Polygenic risk factor (PRF) |
| Vitamin A (Retinol) | rs12934922 rs1667255 rs6564851 | 1.0 1.0 1.0 | 1.3 1.1 1.3 | 1.6 1.2 1.6 | $\frac{\begin{matrix}( rs 12934922 + rs 1667255 + rs 6564851 + \\ rs 7501331 + rs 11645428 )\end{matrix}}{5}$ |
| | rs7501331 | 1.0 | 1.3 | 1.6 | |
| | rs11645428 | 1.0 | 1.3 | 1.6 | |
| Vitamin A (beta-carotene) | rs11645428 | 1.0 | 1.3 | 1.6 | rs11645428 |
| Vitamin B1 (Thiamine diphosphate) | rs17514104 | 1.0 | 1.1 | 1.2 | rs17514104 |
| Vitamin B2 (Riboflavin 5-Phosphate) | rs1799983 | 1.0 | 1.1 | 1.2 | rs1799983 |
| Vitamin B7 (Biotin) | rs13078881 | 1.0 | 1.1 | 1.2 | rs13078881 |
| Vitamin B12 (Cyanocobalamin) | rs492602 rs526934 | 1.0 1.0 | 1.1 1.1 | 1.2 1.2 | $\frac{( rs 492602 + rs 526934 + rs 602662 )}{3}$ |
| | rs602662 | 1.0 | 1.1 | 1.2 | |
| Vitamin C (L-Ascorbic acid) | rs33972313 rs4257763 rs6139591 | 1.0 1.0 1.0 | 1.3 1.3 1.3 | 1.6 1.6 1.6 | $\frac{\begin{matrix}( rs 33972313 + rs 4257763 + \\ rs 6139591 + rs 6596473 )\end{matrix}}{4}$ |
| | rs6596473 | 1.0 | 1.3 | 1.6 | |
| Vitamin D, 25-OH | rs10741657 rs12785878 rs2282679 | 1.0 1.0 1.0 | 1.3 1.1 1.3 | 1.6 1.2 1.6 | $\frac{\begin{matrix}( rs 10741657 + rs 12785878 + \\ rs 2282679 + rs 10766197 )\end{matrix}}{4}$ |
| | rs10766197 | 1.0 | 1.3 | 1.6 | |
| Vitamin D3 (Cholecalciferol) | rs10877012 | 1.0 | 1.3 | 1.6 | rs10877012 |
| Vitamin D, 1-25 dihydroxy | rs4588 | 1.0 | 1.3 | 1.6 | rs4588 |
| Vitamin E (alpha-tocopherol) | rs12272004 | 1.0 | 1.3 | 1.6 | rs12272004 |
| Vitamin K1 (Phylloquinone) | rs2108622 | 1.0 | 0.9 | 0.8 | rs2108622 |
| Folate (L-5-methyltetrahydrofolate) | rs1801131 rs1801133 | 1.0 1.0 | 1.3 1.3 | 1.6 1.6 | $\frac{( rs 1801131 + rs 1801133 )}{2}$ |
| Coenzyme Q10 (Ubiquinone + Ubiquinol), Total | rs775607037 rs786204770 | 1.0 1.0 | 1.1 1.1 | 1.2 1.2 | $\frac{( rs 775607037 + rs 786204770 )}{2}$ |
| Selenium, Se 76 | rs1050450 rs3877899 | 1.0 1.0 | 1.1 1.1 | 1.2 1.2 | $\frac{( rs 1050450 + rs 3877899 )}{2}$ |
| Sodium | rs2304478 rs7204044 | 1.0 1.0 | 0.9 0.9 | 0.8 0.8 | $\frac{( rs 2304478 + rs 7204044 )}{2}$ |
| Potassium | rs889299 | 1.0 | 1.1 | 1.2 | rs889299 |
| Calcium, Ca 44 | rs4516035 | 1.0 | 1.3 | 1.6 | rs4516035 |
| Zinc, Zn 67 | rs11126936 | 1.0 | 1.3 | 1.6 | rs11126936 |
| Manganese, Mn 55 | rs13107325 | 1.0 | 1.1 | 1.2 | rs13107325 |
| Iron, Fe 56 | rs1799945 rs1800562 rs3811647 | 1.0 1.0 1.0 | 0.9 0.9 1.1 | 0.8 0.8 1.2 | $\frac{\begin{matrix}( rs 1799945 + rs 1800562 + rs 3811647 + \\ rs 4820268 + rs 855791 )\end{matrix}}{5}$ |
| | rs4820268 | 1.0 | 1.1 | 1.2 | |
| | rs855791 | 1.0 | 1.1 | 1.2 | |
| Magnesium, Mg 24 | rs4680 | 1.0 | 1.1 | 1.2 | rs4680 |
| Copper, Cu 63 | rs76151636 | 1.0 | 0.9 | 0.8 | rs76151636 |
| Iodine | rs225014 | 1.0 | 1.1 | 1.2 | rs225014 |
| Molybdenum | rs594445 | 1.0 | 1.1 | 1.2 | rs594445 |
| Phosphorus | rs4074995 | 1.0 | 1.1 | 1.2 | rs4074995 |
| Tetrahydrobiopterin | rs5030853 rs8007267 | 1.0 1.0 | 1.3 1.1 | 1.6 1.2 | $\frac{( rs 5030853 + rs 8007267 )}{2}$ |
| Fluoride | rs4284505 | 1.0 | 0.9 | 0.8 | rs4284505 |
| Glutathione Oxidized | rs121909307 rs1695 | 1.0 1.0 | 1.1 1.1 | 1.2 1.2 | $\frac{( rs 121909307 + rs 1695 )}{2}$ |
| MMA (Methylmalonic acid) | rs291466 rs121918252 | 1.0 1.0 | 1.3 1.1 | 1.6 1.2 | $\frac{( rs 291466 + rs 121918252 )}{2}$ |
| Choline | rs3733890 rs7946 | 1.0 1.0 | 1.3 1.3 | 1.6 1.6 | $\frac{( rs 3733890 + rs 7946 )}{2}$ |
| Phenylalanine | rs5030853 | 1.0 | 0.9 | 0.8 | rs5030853 |
| LA (Linoleic acid) | rs174547 | 1.0 | 0.9 | 0.8 | rs174547 |

TABLE 5
Default preference for conditions gauged by the PHQ

| | |
|---|---|
| 1 | Heart conditions |
| 2 | Blood pressure |
| 3 | Bone conditions |
| 4 | High blood sugar |
| 5 | GI discomfort |
| 6 | Renal conditions |
| 7 | Liver conditions |
| 8 | Weight gain |
| 9 | Infections |
| 10 | Skin issues |
| 11 | Pimple susceptibility |
| 12 | Depression |
| 13 | Poor sleep |
| 14 | Poor concentration |

TABLE 6

| | | OUTCOME | |
|---|---|---|---|
| Sr. | | | Supporting |
| No | QUESTIONS FROM THE PHQ | Appropriate Nutrient Supplementation | Supplements |
| 1 | Gender | Can be factored in using the RDA values determined for men and | |
| | | women, respectively (Table 14) | |
| 2 | Height | Can be factored in using the ‘Body Metrics’ factor | |
| 3 | Weight | Can be factored in using the ‘Body Metrics’ factor | |
| 4 | Pregnant/Nursing | If yes, then can be factored in using the RDA values determined for | |
| | | pregnant/nursing women (Table 14) | |
| 5 | Were you previously deficient in any | | |
| | nutrient? | | |
| | (5.1) If B12 then . . . | | |
| | Do you suffer from any of the | | |
| | following B12 deficiency symptoms? | | |
| | (a) Macrocytic anemia | Hydroxocobalamine | |
| | (b) Leber optic atrophy | Hydroxocobalamine | |
| | (c) High homocysteine levels | Methylcobalamin | |
| | (5.2) If B9 then . . . | | |
| | Do you suffer from any of the | | |
| | following B9 deficiency symptoms? | | |
| | (a) High homocysteine levels | L-5-Methyltetrahydrofolate | |
| | (b) Megaloblastic anemia | Folic acid | |
| | (c) Macrocytic anemia | Folic acid | |
| 6 | Are you an athlete? | L-Carnitine Tartrate | Quercetin |
| | | | Phosphatidylserine |
| 7 | Do you suffer from poor sleep? | | Micro PQQ |
| 8 | Do you have difficulties in | Magnesium L-threonate | Micro PQQ |
| | concentrating? | | |
| 9 | Do you suffer from depression, | L-5-methyltetrahydrofolate, calcium salt | Curcumin |
| | anxiety, or mood swings? | | |
| | | | Micro PQQ |
| | | | Berberine Extract |
| | | | Phosphatidylcholine |
| | | | Phosphatidylserine |
| 10 | Do you frequently suffer from flu or | Zinc gluconate | Ginger |
| | infections? | | |
| | | | Quercetin |
| | | | Berberine Extract |
| 11 | Are you experiencing pain in your | | Ginger |
| | bones or joints? | | |
| | | | Curcumin |
| 12 | Do you have sensitive skin? | Inositol Hexanicotinate | |
| 13 | Do you often notice breakouts or | Zinc sulfate | Licorice |
| | pimples on your skin? | | |
| 14 | Are you experiencing excessive | | Berberine Extract |
| | weight gain? | | |
| 15 | Do you suffer from high blood sugar | Magnesium taurate | Micro PQQ |
| | levels? | | |
| | | | Berberine Extract |
| | | | Broccoli |
| 16 | Do you have high blood pressure? | Magnesium taurate | Quercetin |
| 17 | Do you suffer from heart conditions? | Mixed Tocopherols | Ginger |
| | | | Curcumin |
| | | | Berberine Extract |
| | | | Broccoli |
| 18 | Do you frequently experience | Ferrous Bisglycinate Chelate | Ginger |
| | gastrointestinal discomfort? | | |
| | | Magnesium malate | Licorice |
| | | Sodium ascorbate | |
| | | Zinc Carnosine | |
| 19 | Do you suffer from renal conditions? | Potassium citrate | |
| 20 | Do you suffer from liver conditions? | | |
| | | | Phosphatidylcholine |
| 21 | Are you on any medications for heart | Calcium citrate | |
| | conditions, hypertension, or acid | | |
| | reflux? If yes, then please | | |
| | mention . . . | | |
| 22 | Do you experience an energy slump | Vitamin B12 | Green tea extract |
| | after eating food? | | |
| | | Citrulline | |
| 23 | Do you get regular exposure to the | Vitamin D | 5-hydroxy-tryptophan |
| | sun (face, arms and hands for at least | | |
| | 10-15 mins per day)? | | |
| 24 | How often do you exercise? | | |
| 25 | In terms of fitness, are you looking | | |
| | for support for the following: | | |
| | (a) Endurance | L-Isoleucine | Nitrates |
| | | L-Valine | Caffeine |
| | | L-Leucine | Probiotics |
| | | L-Arginine | |
| | | Taurine | |
| | | Vitamin C | |
| | | Vitamin E | |
| | | Beta-carotene | |
| | | Selenium | |
| | | Coenzyme Q10 | |
| | | Manganese | |
| | | Beta-alanine | |
| | (b) Muscle strength | Histidine | |
| | | L-isoleucine | |
| | | L-leucine | |
| | | Lysine | |
| | | L - valine | |
| | | Methionine | |
| | | Phenylalanine | |
| | | Threonine | |
| | | Tryptophan | |
| | (c) Muscle mass | Histidine | |
| | | L-isoleucine | |
| | | L-leucine | |
| | | Lysine | |
| | | L- valine | |
| | | Methionine | |
| | | Phenylalanine | |
| | | Threonine | |
| | | Tryptophan | |
| | | L-Arginine | |
| | | Glycine | |
| | | Vitamin D3 | |
| | | DHA | |
| | | EPA | |
| | (d) Muscle recovery | Vitamin D3 | Curcumin |
| | | Coenzyme Q10 | Ginger |
| | | L-Isoleucine | Quercetin |
| | | L-leucine | Epigallocatechin gallate |
| | | L-Valine | Ginseng |
| | | L-arginine | Rhodiola rosea |
| | | Glycine | β-hydroxy-β- |
| | | | methylbutyrate |
| | | L-methionine | α-ketoisocaproic acid |
| | | Taurine | Caffeine |
| | | Citrulline | Methylsulfony1- |
| | | | methane |
| | | L-glutamine | |
| | | DHA | |
| | | EPA | |

TABLE 7

| Supporting supplement | Condition | Dosage | |
|---|---|---|---|
| Micro PQQ | Poor sleep/Sugar levels/Mood disorders/Memory and cognition | 20 | mg/day |
| Ginger | Bone health/Cardiac conditions/Immune health/GI symptoms/Kidney conditions/ | 500 | mg/day |
| | Muscle recovery/Hepatic conditions | | |
| Curcumin | Bone health/Cardiac conditions/Mood disorders/Muscle recovery/Hepatic conditions | 1.5 | g/day |
| Berberine Extract | Weight/Cardiac conditions/Sugar levels/Immune health/Mood disorders | 400 | mg/day |
| Phosphatidylcholine | Liver conditions/Mood disorders/Cardiac conditions | 840 | mg/day |
| Quercetin | High BP/Immune health/Muscle soreness/Kidney conditions/Muscle recovery | 250 | mg/day |
| | Cardiac Health/Hepatic conditions | | |
| Phosphatidylserine | Mood disorders/Muscle soreness/Cardiac Health | 200 | mg/day |
| Licorice | Acne/GI symptoms/Cardiac Health | 100 | mg/day |
| Broccoli | Cardiac conditions/Sugar levels/Cardiac Health | 50 | mg/day |
| Green tea extract | Cardiac conditions/Energy metabolism/ | 300 | mg/day |
| 5-hydroxy-tryptophan | Poor sun exposure | 100 | mg/day |
| Nitrates | Endurance | 300 | mg/day |
| Caffeine | Endurance/Muscle recovery | 100 | mg/day |
| Probiotics | Gut dysbiosis/Endurance | 10 billion | CFU/day |
| Prebiotics | Gut dysbiosis | 15 | g/day |
| Epigallocatechin gallate | Muscle recovery | 1800 | mg/day |
| Ginseng | Muscle recovery | 1.6 | g/day |
| Rhodiola rosea | Muscle recovery | 60 | mg/day |
| β-hydroxy-β-methylbutyrate | Muscle recovery | 3 | g/day |
| α-ketoisocaproic acid | Muscle recovery | 0.3 | g/day |
| Methylsulfonyl-methane | Muscle recovery | 3 | g/day |
| Betaine | Cardiac conditions | 350 | mg/day |
| Silymarin | Cardiac conditions | 200 | mg/day |
| Resveratrol | Cardiac conditions | 75 | mg/day |
| Lycopene | Cardiac conditions | 6.5 | mg/day |
| Catechin | Cardiac conditions | 500 | mg/day |
| Chitosan | Kidney conditions | 1 | g/day |
| Glucoraphanin | Hepatic conditions | 100 | mg/day |

TABLE 8

| | Genetic | | | | | | |
|---|---|---|---|---|---|---|---|
| | polymorphism | Genotype Score | | | Cumulative | Supporting | |
| Nutrient | (rs ID) | Wild | Het | Mutant | Effect | Supplement | Reason |
| Vitamin B2 | rs1799983 | 1.0 | 1.1 | 1.2 | rs1799983 | Quercetin | The SNP is associated with an increased |
| (Riboflavin | | | | | | | risk of ischemic heart disease |
| 5-Phosphate) | | | | | | | |
| Vitamin B7 | rs13078881 | 1.0 | 1.1 | 1.2 | rs13078881 | Licorice | A vitamin B7 deficiency can cause |
| (Biotin) | | | | | | | skin rashes |
| Vitamin D, 25-OH | rs10741657 rs12785878 rs2282679 | 1.0 1.0 1.0 | 1.3 1.1 1.3 | 1.6 1.2 1.6 | $\frac{\begin{matrix}( rs 10741657 + rs 12785878 + \\ rs 2282679 + rs 10766197 )\end{matrix}}{4}$ | Curcumin, Ginger | These vitamin D SNPs are associated with compromised bone health |
| | rs10766197 | 1.0 | 1.3 | 1.6 | | | |
| Vitamin D3 | rs10877012 | 1.0 | 1.3 | 1.6 | rs10877012 | Curcumin, Ginger | A vitamin D deficiency is associated |
| (Cholecalciferol) | | | | | | | with compromised bone health |
| Vitamin D, 1-25 | rs4588 | 1.0 | 1.3 | 1.6 | rs4588 | Curcumin, Ginger | A vitamin D deficiency is associated |
| dihydroxy | | | | | | | with compromised bone health |
| Folate (L-5- methyl- | rs1801131, rs1801133 | 1.0 1.0 | 1.3 1.3 | 1.6 1.6 | $\frac{( rs 1801131 + rs 1801133 )}{2}$ | Quercetin, Berberine Extract, Broccoli, | A folate deficiency increases the risk of hypertension and cardiovascular |
| tetrahydrofolate) | | | | | | Curcumin, Ginger | diseases |
| Coenzyme Q10 (Ubiquinone + | rs775607037 rs786204770 | 1.0 1.0 | 1.1 1.1 | 1.2 1.2 | $\frac{( rs 775607037 + rs 786204770 )}{2}$ | Ginger, Quercetin, Curcumin, Micro | Coenzyme Q10 is an important antioxidant and its deficiency will |
| Ubiquinol), | | | | | | PQQ, Berberine | reduce the antioxidant pool in the body |
| Total | | | | | | Extract, Broccoli | |
| Selenium, Se 76 | rs1050450 rs3877899 | 1.0 1.0 | 1.1 1.1 | 1.2 1.2 | $\frac{( rs 1050450 + rs 3877899 )}{2}$ | Ginger, Quercetin, Curcumin, Micro | A selenium deficiency can lead to oxidative stress in the body |
| | | | | | | PQQ, Berberine | |
| | | | | | | Extract, Broccoli | |
| Sodium | rs2304478 rs7204044 | 1.0 1.0 | 0.9 0.9 | 0.8 0.8 | $\frac{( rs 2304478 + rs 7204044 )}{2}$ | Quercetin | Sodium mutations causing increased sodium reabsorption can lead to the |
| | | | | | | | risk of hypertension |
| Calcium, Ca 44 | rs4516035 | 1.0 | 1.3 | 1.6 | rs4516035 | Curcumin, Ginger | A calcium deficiency can lower bone |
| | | | | | | | mineral density |
| Zinc, Zn 67 | rs11126936 | 1.0 | 1.3 | 1.6 | rs11126936 | Micro PQQ, | The SNP is associated with impaired |
| | | | | | | Broccoli | transport of zinc to pancreatic B-cells |
| | | | | | | | affecting insulin secretion. Anti- |
| | | | | | | | diabetics can modulate the risk |
| | | | | | | | associated with altered insulin levels |
| Iron, Fe 56 | rs4820268 | 1.0 | 1.1 | 1.2 | rs4820268 | Ginger, Licorice, | The SNP results in impaired iron content |
| | | | | | | Quercetin. Micro | regulation which can lead to iron |
| | | | | | | PQQ, Berberine | deficiency (anaemia). Anaemic patients |
| | | | | | | Extract | may have gastrointestinal disturbances, |
| | | | | | | | impaired cognitive function, immune |
| | | | | | | | function, exercise or work performance, |
| | | | | | | | and body temperature regulation |
| Magnesium, | rs4680 | 1.0 | 1.1 | 1.2 | rs4680 | Micro PQQ, | This SNP is associated with lowered |
| Mg 24 | | | | | | Curcumin, | COMT gene activity which is regulated |
| | | | | | | Phosphatidylcholine, | by magnesium. The outcome of the is |
| | | | | | | Phosphatidylserine | increased dopamine levels in the brain, |
| | | | | | | | that can affect cognitive functions |
| Fluoride | rs4284505 | 1.0 | 0.9 | 0.8 | rs4284505 | Licorice | The SNP is associated with affected |
| | | | | | | | enamel mineralization in the presence |
| | | | | | | | of fluoride leading to fluorosis |
| Iodine | rs225014 | 1.0 | 1.1 | 1.2 | rs225014 | Curcumin, Ginger | The SNP results in affected iodine- |
| | | | | | | | associated gene which is responsible |
| | | | | | | | for maintaining T3. This alteration can |
| | | | | | | | lead to diseases of the bone such as |
| | | | | | | | osteoarthritis |
| Molybdenum | rs594445 | 1.0 | 1.1 | 1.2 | rs594445 | Micro PQQ, | The SNP affects the molybdenum |
| | | | | | | Curcumin | cofactor enzyme resulting in the loss of |
| | | | | | | | function of MOCOS gene that leads to |
| | | | | | | | build up of certain toxic chemicals, |
| | | | | | | | especially in the brain which can affect |
| | | | | | | | brain health |
| Glutathione | rs121909307 | 1.0 | 1.1 | 1.2 | $\frac{( rs 121909307 + rs 1695 )}{2}$ | Ginger, Quercetin, Curcumin, Micro | Glutathione is an important antioxidant and alterations in its levels/functions |
| | | | | | | PQQ, Berberine | will reduce the antioxidant pool in |
| | | | | | | Extract, Broccoli | the body |

TABLE 9

| Nutrient | Nutrient-synthesizing gut bacteria | Exemplary Corrective measure |
|---|---|---|
| Vitamin B1 | Streptococcus thermophilus ST5 | S. thermophilus, L. helveticus, and B. longum are excellent examples of probiotics that |
| (Thiamine) | Lactobacillus helveticus R0052, | can help regain bacterial population in gut. These bacteria may improve Vitamin B1 |
| | B. longum R0175 | deficiency in the gut. |
| Vitamin B2 | Bacillus subtilis | This vitamin B2 synthesizing bacteria can be supplemented as probiotics. They can |
| (Riboflavin) | | help regain the bacterial population and may improve gut-mediated vitamin B2 |
| | | deficiency. |
| Vitamin B3 | Bacteroides fragilis | B. fragilis and R. lactaris are good probiotics. P. copri is recently being used as a |
| (Nicotinic acid) | Prevotella copri, | probiotic. These bacteria can help regain the bacterial population and may improve |
| | Ruminococcus lactaris | gut-mediated vitamin B3 deficiency. |
| Vitamin B5 | Corynebacterium glutamicum | This vitamin B5 synthesizing bacteria can be supplemented as probiotics. They can |
| (Pantothenic acid | | help regain the bacterial population and may improve gut-mediated vitamin B5 |
| | | deficiency. |
| Vitamin B6 | Streptococcus thermophilus ST5 | S. thermophilus, L. helveticus, and B. longum are excellent examples of probiotics |
| (Pyridoxine) | Lactobacillu shelveticus R0052, | that can help regain bacterial population in gut. These bacteria may improve |
| | B. longum R0175 | Vitamin B6 deficiency in the gut. |
| Vitamin B7 | Bacteroides fragilis | B. fragilis and F. varium are good probiotics. P. copri is recently being used as a |
| (Biotin) | Prevotella copri | probiotic. These bacteria can help regain the bacterial population and may improve |
| | Fusobacterium varium | gut-mediated vitamin B7 deficiency. |
| Vitamin B9 | Bifidobacterium breve, | Bifidobacterium are part of the normal gut flora and can be consumed as probiotic |
| (Folate) | Bifidobacterium longum subsp. longum | supplements. They can help regain the bacterial population and may improve gut- |
| | Bifidobacterium adolescentis | mediated vitamin B9 deficiency. |
| Vitamin B12 | P. freudenreichii | P. freudenreichii and L. reuteri are well-known and well-studied probiotics that can |
| (Cobalamin) | Lactobacillus reuteri | be safely consumed as supplements. They can help regain the bacterial population |
| | | and may improve gut-mediated vitamin B12 deficiency. |
| Vitamin D, | Lactobacillus reuteri | L. reuteri are well-known and well-studied probiotics that can be safely consumed as |
| 1-25 dihydroxy | | supplements. They can help regain the bacterial population and may improve gut- |
| | | mediated vitamin D deficiency. |
| Vitamin K2 | Eubacterium lentum | These vitamin K2 synthesizing bacteria can be supplemented as probiotics. They can |
| (Menaquinone- | Veillonella | help regain the bacterial population and may improve gut-mediated vitamin K2 |
| MK-7) | | deficiency. |
| L-Glutamine | Bacteroides, Bifidobacterium, Eubacterium, | Bacteroides and Bifidobacterium are well studied probiotics. Lachnospiraceae is a |
| | Lachnospiraceae, Ruminococcaceae. | next generation probiotic. These bacteria can help regain the bacterial population and |
| | | may improve gut-mediated L-Glutamine deficiency. |
| L-Serine | Clostridium acetobutylicum | Clostridium species are excellent probiotics and can help regain the bacterial |
| | | population and may improve gut-mediated L-serine deficiency. |
| L-Arginine | Bacteroidetes | These L-arginine synthesizing bacteria can be supplemented as probiotics. They can |
| | Firmicutes | help regain the bacterial population and may improve gut-mediated L-Arginine |
| | | deficiency. |
| L-Isoleucine | Corynebacterium glutamicum | These L-Isoleucine synthesizing bacteria can be supplemented as probiotics. They |
| | | can help regain the bacterial population and may improve gut-mediated L- |
| | | Isoleucine deficiency |
| Phenylalanine | Clostridium sporogenes | Clostridium species are excellent probiotics and can help regain the bacterial |
| | | population and may improve gut-mediated phenylalanine deficiency. |

TABLE 10
Cardiovascular Health

| | | Dosage | | | | | Dosage | | | |
|---|---|---|---|---|---|---|---|---|---|---|
| Biomarker | Nutrient | Men | | Women | | Supporting Supplements | Men | | Women | |
| Cholesterol | Vitamin B3 | 16 | mg/day | 14 | mg/day | Ginger | 500 | mg/day | 500 | mg/day |
| | Vitamin C | 90 | mg/day | 75 | mg/day | Curcumin | 1.5 | g/day | 1.5 | g/day |
| | | | | | | Berberine | 400 | mg/day | 400 | mg/day |
| | | | | | | Phosphatidylcholine (PC) | 840 | mg/day | 840 | mg/day |
| | | | | | | Licorice | 100 | mg/day | 100 | mg/day |
| | | | | | | Broccoli | 50 | mg/day | 50 | mg/day |
| HDL Direct | Vitamin B3 | 16 | mg/day | 14 | mg/day | Curcumin | 1.5 | g/day | 1.5 | g/day |
| | DHA | 1 | g/day | 1 | g/day | Berberine | 400 | mg/day | 400 | mg/day |
| | | | | | | Phosphatidylcholine (PC) | 840 | mg/day | 840 | mg/day |
| | | | | | | Phosphatidylserine (PS) | 200 | mg/day | 200 | mg/day |
| LDL Direct | Vitamin B3 | 16 | mg/day | 14 | mg/day | Curcumin | 1.5 | g/day | 1.5 | g/day |
| | DHA | 1 | g/day | 1 | g/day | Broccoli | 50 | mg/day | 50 | mg/day |
| Triglycerides | Vitamin C | 90 | mg/day | 75 | mg/day | Curcumin | 1.5 | g/day | 1.5 | g/day |
| | Vitamin B3 | 16 | mg/day | 14 | mg/day | Broccoli | 50 | mg/day | 50 | mg/day |
| | DHA | 1 | g/day | 1 | g/day | Quercetin | 250 | mg/day | 250 | mg/day |
| | EPA | 1 | g/day | 1 | g/day | | | | | |
| Apolipoprotein | Vitamin D | 600 | IU/day | 600 | IU/day | Curcumin | 1.5 | g/day | 1.5 | g/day |
| A-1 | | | | | | | | | | |
| | Vitamin B3 | 16 | mg/day | 14 | mg/day | Berberine | 400 | mg/day | 400 | mg/day |
| | | | | | | Phosphatidylcholine (PC) | 840 | mg/day | 840 | mg/day |
| | | | | | | Phosphatidylserine (PS) | 200 | mg/day | 200 | mg/day |
| Apolipoprotein | Vitamin B3 | 16 | mg/day | 14 | mg/day | Curcumin | 1.5 | g/day | 1.5 | g/day |
| B | | | | | | | | | | |
| | Vitamin C | 90 | mg/day | 75 | mg/day | | | | | |
| PLAC | Vitamin B3 | 16 | mg/day | 14 | mg/day | | | | | |
| | DHA | 1 | g/day | 1 | g/day | | | | | |
| | EPA | 1 | g/day | 1 | g/day | | | | | |
| | Alpha | 50 | mg/day | 50 | mg/day | | | | | |
| | Lipoic Acid | | | | | | | | | |
| Homocysteine | Folic Acid | 400 | mcg/day | 400 | mcg/day | Curcumin | 1.5 | g/day | 1.5 | g/day |
| | Vitamin B2 | 1.3 | mg/day | 1.1 | mg/day | Quercetin | 250 | mg/day | 250 | mg/day |
| | Vitamin B6 | 1.3 | mg/day | 1.3 | mg/day | Phosphatidylcholine (PC) | 840 | mg/day | 840 | mg/day |
| | Vitamin | 2.4 | mcg/day | 2.4 | mcg/day | Berberine | 400 | mg/day | 400 | mg/day |
| | B12 | | | | | | | | | |
| | | | | | | Betaine | 350 | mg/day | 350 | mg/day |
| hs-CRP | DHA | 1 | g/day | 1 | g/day | Curcumin | 1.5 | g/day | 1.5 | g/day |
| | EPA | 1 | g/day | 1 | g/day | Silymarin | 200 | mg/day | 200 | mg/day |
| | Magnesium | 400 | mg/day | 310 | mg/day | | | | | |
| | Vitamin C | 90 | mg/day | 75 | mg/day | | | | | |
| | Vitamin D | 600 | IU/day | 600 | IU/day | | | | | |
| | Vitamin E | 22 | IU/day | 22 | IU/day | | | | | |
| Oxidised LDL | Vitamin B3 | 16 | mg/day | 14 | mg/day | Quercetin | 250 | mg/day | 250 | mg/day |
| | Tocotrienols | 22 | IU/day | 22 | IU/day | Curcumin | 1.5 | g/day | 1.5 | g/day |
| | Glutathione | 500 | mg/day | 500 | mg/day | Resveratrol | 75 | mg/day | 75 | mg/day |
| | | | | | | Green tea extract | 300 | mg/day | 300 | mg/day |
| | | | | | | Lycopene | 6.5 | mg/day | 6.5 | mg/day |
| | | | | | | Catechin | 500 | mg/day | 500 | mg/day |
| MPO | Taurine | 500 | mg/day | 500 | mg/day | | | | | |

TABLE 11
Neural Health Test

| | | Dosage | | | | Supporting | Dosage | |
|---|---|---|---|---|---|---|---|---|
| Biomarker | Nutrient | Men | | Women | | Supplements | Men | Women |
| Amyloid | Vitamin A | 3,000 | IU/day | 2,333 | IU/day | | | |
| beta | | | | | | | | |
| | Beta- | 6 | mg/day | 6 | mg/day | | | |
| | carotene | | | | | | | |
| | Vitamin B6 | 1.3 | mg/day | 1.3 | mg/day | | | |
| | Vitamin B9 | 400 | mcg/day | 400 | mcg/day | | | |
| | Vitamin C | 90 | mg/day | 75 | mg/day | | | |
| | Vitamin E | 22 | IU/day | 22 | IU/day | | | |
| Tau | Vitamin | 2.4 | mcg/day | 2.4 | mcg/day | | | |
| | B12 | | | | | | | |
| BDNF | Zinc | 11 | mg/day | 8 | mg/day | Curcumin | 1.5 g/day | 1.5 g/day |
| | Iron | 8 | mg/day | 18 | mg/day | | | |

TABLE 12
Thyroid Panel

| | | Dosage | | | |
|---|---|---|---|---|---|
| Biomarker | Nutrient | Men | | Women | |
| T4 | Zinc | 11 | mg/day | 8 | mg/day |
| | Copper | 900 | mcg/day | 900 | mcg/day |
| | Calcium | 1,000 | mg/day | 1,000 | mg/day |
| | Iron | 8 | mg/day | 18 | mg/day |
| | Valine | 24 | mg/kg/day | 24 | mg/kg/day |
| | Leucine | 42 | mg/kg/day | 42 | mg/kg/day |
| | Arginine | 1000 | mg/day | 1000 | mg/day |
| | Iodine | 150 | mcg/day | 150 | mcg/day |
| | Selenium | 55 | mcg/day | 55 | mcg/day |
| | Citrulline | 100 | mg/kg/day | 100 | mg/kg/day |
| | Choline | 550 | mg/day | 425 | mg/day |
| fT4 | Zinc | 11 | mg/day | 8 | mg/day |
| | Copper | 900 | mcg/day | 900 | mcg/day |
| | Calcium | 1,000 | mg/day | 1,000 | mg/day |
| | Iron | 8 | mg/day | 18 | mg/day |
| | Valine | 24 | mg/kg/day | 24 | mg/kg/day |
| | Leucine | 42 | mg/kg/day | 42 | mg/kg/day |
| | Arginine | 1000 | mg/day | 1000 | mg/day |
| | Choline | 550 | mg/day | 425 | mg/day |
| TSH | Zinc | 11 | mg/day | 8 | mg/day |
| | Vitamin D | 600 | IU/day | 600 | IU/day |
| | Vitamin A | 3,000 | IU/day | 2,333 | IU/day |
| | Serine | 500 | mg/day | 500 | mg/day |
| | Vitamin B9 | | | | |
| T3 | Arginine | 1000 | mg/day | 1000 | mg/day |
| | Copper | 900 | mcg/day | 900 | mcg/day |
| | Iodine | 150 | mcg/day | 150 | mcg/day |
| | Vitamin D | 600 | IU/day | 600 | IU/day |
| | Selenium | 55 | mcg/day | 55 | mcg/day |
| fT3 | Calcium | 1,000 | mg/day | 1,000 | mg/day |
| | Selenium | 55 | mcg/day | 55 | mcg/day |
| | Vitamin E | 22 | IU/day | 22 | IU/day |
| | Vitamin D | 600 | IU/day | 600 | IU/day |
| | Iron | 8 | mg/day | 18 | mg/day |
| rT3 | Selenium | 55 | mcg/day | 55 | mcg/day |
| Anti-TPO | Vitamin D | 600 | IU/day | 600 | IU/day |
| | Carnitine | 500 | mg/day | 500 | mg/day |
| | Vitamin B9 | 400 | mcg/day | 400 | mcg/day |
| Anti-Tg | Carnitine | 500 | mg/day | 500 | mg/day |
| | Vitamin B9 | 400 | mcg/day | 400 | mcg/day |

TABLE 13
Kidney Health

| | | Dosage | | Supporting | Dosage | | | |
|---|---|---|---|---|---|---|---|---|
| Biomarker | Nutrient | Men | Women | Supplements | Men | | Women | |
| BUN/creatinine | | | | Quercetin | 250 | mg/day | 250 | mg/day |
| Calcium | Vitamin D | 600 IU/day | 600 IU/day | | | | | |
| Glucose | | | | Ginger | 500 | mg/day | 500 | mg/day |
| Phosphate, | Vitamin D | 600 IU/day | 600 IU/day | | | | | |
| Inorganic | | | | | | | | |
| Creatinine | | | | Chitosan | 1 | g/day | 1 | g/day |
| Albumin | | | | Quercetin | 250 | mg/day | 250 | mg/day |

TABLE 14
Hepatic Function Panel

| | | Dosage | | | | Supporting | Dosage | | | |
|---|---|---|---|---|---|---|---|---|---|---|
| Biomarker | Nutrient | Men | | Women | | Supplements | Men | | Women | |
| Bilirubin | Iron | 8 | mg/day | 18 | mg/day | | | | | |
| | Zinc | 11 | mg/day | 8 | mg/day | | | | | |
| | Copper | 900 | mcg/day | 900 | mcg/day | | | | | |
| ALT | Vitamin E | 22 | IU/day | 22 | IU/day | Curcumin | 1.5 | g/day | 1.5 | g/day |
| | NAC (N-acetyl | 19 | mg/kg/day | 19 | mg/kg/day | Quercetin | 250 | mg/day | 250 | mg/day |
| | cysteine) | | | | | | | | | |
| | | | | | | Glucoraphanin | 100 | mg/day | 100 | mg/day |
| ALP | Vitamin C | 90 | mg/day | 75 | mg/day | Curcumin | 1.5 | g/day | 1.5 | g/day |
| ASP | Vitamin E | 22 | IU/day | 22 | IU/day | Ginger | 500 | mg/day | 500 | mg/day |

TABLE 15

| | | RDA | | | | |
|---|---|---|---|---|---|---|
| # | Nutrient | Men | Women | Pregnancy | Lactating | UTL |
| | Vitamin A (Retinol) | 3,000 | 2,333 | | | 10,000 |
| | | IU/day | IU/day | | | IU/day |
| | Vitamin B1 (Thiamine | 1.2 | 1.1 | 1.4 | 1.4 | * |
| | diphosphate) | mg/day | mg/day | mg/day | mg/day | |
| | Vitamin B2 (Riboflavin | 1.3 | 1.1 | 1.4 | 1.6 | * |
| | 5-Phosphate) | mg/day | mg/day | mg/day | mg/day | |
| | Vitamin B3 (Nicotinic | 16 | 14 | 18 | 17 | 35 |
| | acid) | mg/day | mg/day | mg/day | mg/day | mg/day |
| | Vitamin B5 (Pantothenic | 5 | 5 | 6 | 7 | * |
| | acid) | mg/day | mg/day | mg/day | mg/day | |
| | Vitamin B6, Pyridoxal 5- | 1.3 | 1.3 | 1.9 | 2.0 | 100 |
| | Phosphate | mg/day | mg/day | mg/day | mg/day | mg/day |
| | Vitamin B12 | 2.4 | 2.4 | 2.6 | 2.8 | * |
| | (Cyanocobalamin) | mcg/day | mcg/day | mcg/day | mcg/day | |
| | Vitamin C (L-Ascorbic | 90 | 75 | 85 | 120 | 2000 |
| | acid) | mg/day | mg/day | mg/day | mg/day | mg/day |
| | Vitamin D, 25-OH | 600 | 600 | 600 | 600 | * |
| | | IU/day | IU/day | IU/day | IU/day | |
| 0 | Vitamin D3 | 600 | 600 | 600 | 600 | * |
| | (Cholecalciferol) | IU/day | IU/day | IU/day | IU/day | |
| 1 | Vitamin E (alpha- | 22 | 22 | 15 | 19 | 1,100 |
| | tocopherol) | IU/day | IU/day | mg/day | mg/day | IU/day |
| 2 | Vitamin K1 | 120 | 90 | 90 | 90 | * |
| | (Phylloquinone) | mcg/day | mcg/day | mcg/day | mcg/day | |
| 3 | Vitamin K2 | 120 | 90 | 90 | 90 | * |
| | (Menaquinone-MK-7) | mcg/day | mcg/day | mcg/day | mcg/day | |
| 4 | Folate (L-5- | 400 | 400 | 600 | 500 | 1,000 |
| | methyltetrahydrofolate) | mcg/day | mcg/day | mcg/day | mcg/day | mcg/day |
| 5 | Coenzyme Q10 | 100-200 | 100-200 | 200 | 200 | 1,200 |
| | (Ubiquinone + | mg/day | mg/day | mg/day | mg/day | mg/day |
| | Ubiquinol), Total | | | | | |
| 6 | Selenium, Se 76 | 55 | 55 | 60 | 70 | 400 |
| | | mcg/day | mcg/day | mcg/day | mcg/day | mcg/day |
| 7 | Sodium | 1,500 | 1,500 | 1,500 | 1,500 | 2,300 |
| | | g/day | g/day | g/day | g/day | mg/day |
| 8 | Potassium | 3,400 | 2,600 | 2,900 | 2,800 | * |
| | | mg/day | mg/day | mg/day | mg/day | |
| 9 | Calcium, Ca 44 | 1,000 | 1,000 | 1,000 | 1,000 | 2,500 |
| | | mg/day | mg/day | mg/day | mg/day | mg/day |
| 0 | Zinc, Zn 67 | 11 | 8 | 11 | 12 | 40 |
| | | mg/day | mg/day | mg/day | mg/day | mg/day |
| 1 | Manganese, Mn 55 | 2.3 | 1.8 | 2.0 | 2.6 | 11 |
| | | mg/day | mg/day | mg/day | mg/day | mg/day |
| 2 | Iron, Fe 56 | 8 | 18 | 27 | 9 | 45 |
| | | mg/day | mg/day | mg/day | mg/day | mg/day |
| 3 | Magnesium, Mg 24 | 400 | 310 | 350 | 310 | 350 |
| | | mg/day | mg/day | mg/day | mg/day | mg/day |
| 4 | Copper, Cu 63 | 900 | 900 | 1,000 | 1,300 | 10 |
| | | mcg/day | mcg/day | mcg/day | mcg/day | mg/day |
| 5 | Chromium, Cr 53 | 35 | 25 | 30 | 45 | * |
| | | mcg/day | mcg/day | mcg/day | mcg | |
| 6 | Choline | 550 | 425 | 450 | 550 | 3,500 |
| | | mg/day | mg/day | mg/day | mg/day | mg/day |
| 7 | L-Cysteine | 19 | 19 | | | * |
| | | mg/kg/day | mg/kg/day | | | |
| 8 | L-Glutamine | 500 | 500 | | | 14000 |
| | | mg/day | mg/day | | | mg/day |
| 9 | L-Serine | 500 | 500 | | | 12000 |
| | | mg/day | mg/day | | | mg/day |
| 0 | L-Arginine | 1000 | 1000 | | | 20000 |
| | | mg/day | mg/day | | | mg/day |
| 1 | L-Isoleucine | 19 | 19 | | | * |
| | | mg/kg/day | mg/kg/day | | | |
| 2 | L-Valine | 24 | 24 | | | * |
| | | mg/kg/day | mg/kg/day | | | |
| 3 | L-Leucine | 42 | 42 | | | ~35 |
| | | mg/kg/day | mg/kg/day | | | g/d |
| 4 | Free Carnitine | 500 | 500 | | | 2000 |
| | | mg/day | mg/day | | | mg/day |
| 5 | DHA (Docosahexaenoic | 1 | 1 | 300 | 300 | * |
| | acid) | g/day | g/day | mg/day | mg/day | |
| 6 | EPA (Eicosapentaenoic | 1 | 1 | 325 | 325 | * |
| | acid) | g/day | g/day | mg/day | mg/day | |

* There are no established UTL values for these nutrients. Thus, safe advisable limits will be considered for these nutrients post consultation with healthcare experts (physicians and dieticians) in the field.

TABLE 16

| | Nutrient | Nutrient Default Form |
|---|---|---|
| Vitamins | Vitamin A (Retinol) | Vitamin A Palmitate |
| and | Vitamin A (beta-carotene) | Beta Carotene |
| minerals | Vitamin B1 (Thiamine diphosphate) | Thiamine Mononitrate |
| | Vitamin B2 (Riboflavin 5-Phosphate) | Riboflavin 5 Phosphate |
| | Vitamin B3 (Nicotinic acid) | Nicotinic Acid |
| | Vitamin B5 (Pantothenic acid) | Calcium Pantothenate |
| | Vitamin B6, Pyridoxal 5-Phosphate | Pyridoxine HCI |
| | Vitamin B7 (Biotin) | Biotin |
| | Vitamin B12 (Cyanocobalamin) | Cyanocobalamin |
| | Vitamin C (L-Ascorbic acid) | Ascorbic Acid |
| | Vitamin D, 25-OH | Cholecalciferol |
| | Vitamin D3 (Cholecalciferol) | Cholecalciferol |
| | Vitamin D, 1-25 dihydroxy | Cholecalciferol |
| | Vitamin E (alpha-tocopherol) | d-Alpha Tocopheryl Succinate |
| | Vitamin K1 (Phylloquinone) | Vitamin K1 |
| | Vitamin K2 (Menaquinone-MK-7) | Vitamin K2 (as Menaquinone-7) |
| | Folate (L-5-methyltetrahydrofolate) | Folinic Acid (Folate) |
| | Coenzyme Q10 (Ubiquinone + | Ubiquinone |
| | Ubiquinol), Total | |
| | Selenium, Se 76 | L-Selenomethionine |
| | Sodium | Sea Salt |
| | Potassium | Potassium chloride |
| | Calcium, Ca 44 | Calcium carbonate |
| | Zinc, Zn 67 | Zinc Picolinate |
| | Manganese, Mn 55 | Manganese Glycinate |
| | Iron, Fe 56 | Ferrous sulfate |
| | Magnesium, Mg 24 | Magnesium Citrate |
| | Copper, Cu 63 | Copper Bisglycinate Chelate |
| | Chromium, Cr 53 | Chromium picolinate |
| | Myo-Inositol | Myo-inositol |
| | Iodine | Potassium Iodide |
| | Molybdenum | Molybdenum Glycinate Chelate |
| | Phosphorus | Dipotassium phosphate |
| | Tetrahydrobiopterin | |
| | Fluoride | |
| | Copper/Zinc | |
| Amino | Glutathione Oxidized | L-Glutathione (reduced) |
| Acids | MMA (Methylmalonic acid) | Cyanocobalamin |
| | Choline | Choline bitartrate |
| | L-Cysteine | N-Acetyl-L-Cysteine (NAC) |
| | L-Asparagine | L-Asparagine |
| | L-Glutamine | L-Glutamine |
| | L-Serine | L-Serine |
| | L-Arginine | L-Arginine |
| | L-Citrulline | L-Citrulline |
| | L-Isoleucine | L-Isoleucine |
| | L-Valine | L-Valine |
| | L-Leucine | L-Leucine |
| | Free Carnitine | L-carnitine |
| | Phenylalanine | L-Phenylalanine |
| Fatty | DHA (Docosahexaenoic acid) | DHA |
| Acids | EPA (Eicosapentaenoic acid) | EPA |
| | DPA (Docosapentaenoic acid) | |
| | AA (Arachidonic acid) | Arachidonic Acid |
| | LA (Linoleic acid) | Conjugated Linoleic Acid |
| | Omega-3 Total | Omega-3 DHA/EPA (High DHA) |
| | | 3:1 |
| | Omega-6 Total | |
| | Omega-3 Index | |
| | AA/EPA | |

TABLE 17

| Nutrient pairings | Reason |
|---|---|
| Synergistic nutrient | |
| pairings | |
| Calcium and vitamin D | Calcium and vitamin D, together help in protecting bones. Vitamin D helps |
| | the body to effectively absorb calcium. |
| Magnesium and vitamin D | Magnesium assists in the activation of vitamin D, and all of the enzymes that |
| | metabolize vitamin D require magnesium, as a cofactor in the enzymatic |
| | reactions in the liver and kidneys. |
| Omega 3 and vitamin E | Omega 3 and vitamin E are important for heart health as they can improve |
| | cholesterol levels. Also, the co-administration of omega 3 and vitamin E |
| | beneficially decreased serum insulin and insulin resistance in CAD patients. |
| Sodium and Potassium | Potassium helps manage the levels of sodium in the body, which is generally |
| | consumed at higher levels. Potassium promotes the excretion of sodium via |
| | the kidney, which lowers sodium levels, thus protecting against hypertension. |
| Folate and Vitamin B12 | Folate depends on vitamin B12 to be absorbed, stored, and metabolized. As a |
| | result, a vitamin B12 deficiency can lead to a folate deficiency. Lastly, they |
| | work together to support processes like cell division and replication. |
| Vitamin B3 (Niacin) and | The amino acid tryptophan helps in the production of niacin. Thus, a |
| tryptophan | tryptophan deficiency can lead to a niacin deficiency. |
| Vitamin D and Omega-3s | Vitamin D and Omega-3s are seen to have a synergistic effect as both |
| | nutrients were more readily absorbed when they were supplemented together. |
| Vitamin C and iron | Vitamin C enhances non-heme iron (iron from plant-based sources) |
| | absorption. It also regulates iron metabolism by stimulating ferritin synthesis, |
| | inhibiting lysosomal ferritin degradation, and decreasing cellular iron efflux. |
| Vitamin A and zinc | Zinc is essential for the enzyme that converts retinol (vitamin A) into retinal |
| | and is also necessary for the hepatic synthesis and secretion of transthyretin, |
| | the protein responsible for transporting vitamin A. Therefore, zinc intake can |
| | be supplemented alongside vitamin A to optimize vitamin A levels and |
| | function. |
| Phosphorus and vitamin D | Since low levels of vitamin D can impair intestinal phosphorus absorption, |
| | phosphorus supplementation may be considered to compensate for reduced |
| | phosphorus absorption due to vitamin D deficiency. |
| Vitamin A and iron | Vitamin A supplementation alongside iron may be beneficial as it can |
| | facilitate the mobilization of iron from storage sites to developing red blood |
| | cells (RBCs) for incorporation into hemoglobin in RBCs. This action can |
| | enable better transportation of iron, thereby optimizing its levels. |
| Zinc and iron | Zinc is crucial for erythropoiesis, and low levels of zinc can exacerbate iron- |
| | deficiency anemia. Since zinc is necessary for proper iron function and for |
| | alleviating conditions caused by low iron levels, it is recommended to |
| | supplement zinc along with iron. |
| Antagonistic nutrient | |
| parings | |
| Iron and zinc | Intestinal absorption of iron and zinc occurs via the same protein, with zinc |
| | being predominantly absorbed. Thus, high levels of zinc supplementation will |
| | not allow iron to be absorbed by the body. |
| Zinc and magnesium | High doses of zinc supplementation (142 mg/day) might interfere with |
| | magnesium absorption and disrupt magnesium balance. |
| Copper and zinc | Copper and zinc compete for absorption sites in the small intestine. As a |
| | result, if zinc levels are excessively high, copper tends to lose out and a |
| | copper deficiency may develop. |
| Calcium and iron | Studies have shown that calcium can inhibit iron absorption, regardless of |
| | whether it is given as Ca salts or in dairy products. This is possibly due to |
| | calcium affecting the uptake of iron through DMT1 (divalent metal |
| | transporter 1) in the intestinal lumen. |
| Vitamin E and vitamin K | A study showed that high doses of vitamin E (antioxidant) may have |
| | antagonistic effects on vitamin K (blood coagulation). |
| Vitamin E and vitamin K | Since vitamin A appears to interfere with vitamin K absorption, oral intake of |
| | vitamin A should not be combined with vitamin K. |
| Iron and manganese | As iron can decrease blood manganese concentrations due to competitive |
| | absorption and transport, oral intake of iron should not be combined with |
| | manganese. |
| Magnesium and manganese | As magnesium can decrease manganese bioavailability by reducing its |
| | absorption or increasing its excretion, oral intake of magnesium along with |
| | manganese is not recommended. |
| Calcium and manganese | As calcium can decrease manganese bioavailability owing to their nutrient |
| | interactions, oral intake of calcium along with manganese is not |
| | recommended. |

TABLE 18

| | | | | | | Target | Customized | | |
|---|---|---|---|---|---|---|---|---|---|
| Level | | Pre-test | | Post-test | | Nutrient | blend | | Follow-up |
| (18-year- | | biomarker | | biomarker | | Value | ((TNV-Post | | Biomarker |
| old Male) | Nutrient | level | ATB | level | VAF | (TNV) | test)/VAF) | UTL | level |
| Cells | Vitamin C | 0.05 | 15 | 1.1 | 0.07 | 8 | 98.57 | 2000 | 9.2 |
| | | pg/MM WBC | mg/day | pg/MM WBC | | pg/MM WBC | mg/day | mg/day | pg/MM WBC |
| | Vitamin B12 | 0.2 | 0.4 | 1.7 | 3.75 | 10 | 2.21 | ND | 10.3 |
| | | | mcg/day | | | | mcg/day | | |
| | Zinc | 2.8 | 1.5 | 4.8 | 1.33333333 | 13.5 | 6.52 | 40 | 14.1 |
| | | ng/MM WBC | mg/day | ng/MM WBC | | ng/MM WBC | mg/day | mg/day | ng/MM WBC |
| | Vitamin D3 | 12.2 | 100 | 18.9 | 0.067 | 200 | 2702.98 | ND | 212 |
| | | pg/MM WBC | IU/day | pg/MM WBC | | pg/MM WBC | IU/day | | pg/MM WBC |
| | EPA | 0.1 | 5 | 0.87 | 0.154 | 2 | 7.33 | ND | 2.1 |
| | | | mg/day | | | | mg/day | | |
| Serum | Vitamin B12 | 105 | 0.4 | 205 | 250 | 1100 | 3.58 | ND | 1170 |
| | | ng/L | mcg/day | ng/L | | ng/L | mcg/day | | ng/L |
| | Calcium | 6.5 | 1 | 7.3 | 0.8 | 10.32 | 3.775 | 2,500 | 10.4 |
| | | mg/dL | mg/day | mg/dL | | mg/dL | mg/day | mg/day | mg/dL |
| | L-Leucine | 82 | 3.5 | 101 | 5.42857143 | 215 | 21 | 35 | 222 |
| | | nmol/m | mg/kg/day | nmol/m | | nmol/m | mg/kg/day | g/d | nmol/m |
| | Vitamin D3 | 0.5 | 100 | 2.3 | 0.018 | 8.5 | 344.44 | ND | 8.7 |
| | | ng/mL | IU/day | ng/mL | | ng/mL | IU/day | | ng/mL |
| | Folate | 1.2 | 50 | 2.8 | 0.032 | 12.5 | 303.12 | 1,000 | 14.3 |
| | | ng/mL | mcg/day | ng/mL | | ng/mL | mcg/day | mcg/day | ng/mL |
| | Coenzyme 10 | 0.2 | 5 | 0.32 | 0.024 | 1.7 | 57.5 | 1,200 | 1.8 |
| | | mcg/mL | mg/day | mcg/mL | | mcg/mL | mg/day | mg/day | mcg/mL |
| | Selenium | 140 | 7.5 | 167 | 3.6 | 255 | 24.44 | 400 | 259 |
| | | ng/mL | mcg/day | ng/mL | | ng/mL | mcg/day | mcg/day | ng/mL |
| | L-Glutamine | 295.3 | 5 | 312 | 3.34 | 650 | 101.20 | 14000 | 697 |
| | | nmol/mL | mg/day | nmol/mL | | nmol/mL | mg/day | mg/day | nmol/mL |
| | Carnitine | 9.5 | 5 | 14.9 | 1.08 | 41 | 24.16 | 2000 | 42.5 |
| | | nmol/mL | mg/day | nmol/mL | | nmol/mL | mg/day | mg/day | nmol/mL |
| | Magnesium | 0.5 | 100 | 1.7 | 0.012 | 2.45 | 62.5 | 350 | 2.52 |
| | | mg/dL | mg/day | mg/dL | | mg/dL | mg/day | mg/day | mg/dL |
| | Copper | 0.2 | 0.08 | 0.9 | 8.75 | 1.65 | 0.09 | 10 | 1.69 |
| | | mcg/mL | mg/day | mcg/mL | | mcg/mL | mg/day | mg/day | mcg/mL |
| | Vitamin K2 | 0.01 | 10 | 0.017 | 0.0007 | 0.10164 | 120.91 | ND | 0.10167 |
| | | ng/ml | mcg/day | ng/ml | | ng/ml | mcg/day | | ng/ml |
| | Vitamin E | 5.5 | 3 | 18.2 | 4.23333333 | 34.5 | 3.85 | 1,100 | 36.5 |
| | | mg/L | IU/day | mg/L | | mg/L | IU/day | IU/day | mg/L |
| | Vitamin B3 | 1.1 | 4 | 3.9 | 0.7 | 65 | 87.28 | 35 | 70.1 |
| | | ng/mL | mg/day | ng/mL | | ng/mL | mg/day | mg/day | ng/mL |
| | L-Valine | 12 | 4 | 47 | 8.75 | 310 | 30.06 | ND | 322 |
| | | nmol/mL | mg/kg/day | mol/mL | | nmol/mL | mg/kg/day | | nmol/mL |
| Genetics | Vitamin C | Heterozygous | | | | | | | |
| | | mutant | | | | | | | |
| | Zinc | Homozygous | | | | | | | |
| | | mutant | | | | | | | |
| | Folate | Homozygous | | | | | | | |
| | | mutant | | | | | | | |
| | Coenzyme 10 | Homozygous | | | | | | | |
| | | mutant | | | | | | | |
| Question- | Arginine | 135 | 10 | 162 | 2.7 | 225 | 23.33 | 20000 | 234 |
| naire | | nmol/mL | mg/kg/day | nmol/mL | | nmol/mL | mg/kg/day | mg/day | nmol/mL |

TABLE 19

| | | | | | | Target | Customized | | |
|---|---|---|---|---|---|---|---|---|---|
| Level | | Pre-test | | Post-test | | Nutrient | blend | | Follow-up |
| (50-year-old | | biomarker | | biomarker | | Value | ((TNV-Post | | Biomarker |
| Female) | Nutrient | level | ATB | level | VAF | (TNV) | test)/VAF) | UTL | level |
| Cells | Vitamin A | 0.2 | 1,000 | 2.1 | 0.0019 | 13.5 | 6000 | 10,000 | 14.5 |
| | | pg/MM | IU/day | pg/MM | | pg/MM | IU/day | IU/day | pg/MM |
| | | WBC | | WBC | | WBC | | | WBC |
| | Manganese | 0.5 | 0.95 | 7.8 | 7.68421053 | 62.5 | 7.11 | 11 | 67.5 |
| | | pg/MM | mg/day | pg/MM | | pg/MM | mg/day | mg/day | pg/MM |
| | | WBC | | WBC | | WBC | | | WBC |
| | Vitamin C | 0.1 | 15 | 1.1 | 0.06666667 | 8 | 103.5 | 2000 | 8.3 |
| | | pg/MM | mg/day | pg/MM | | pg/MM | mg/day | mg/day | pg/MM |
| | | WBC | | WBC | | WBC | | | WBC |
| | Vitamin D3 | 12.2 | 100 | 18.9 | 0.067 | 200 | 2702.98 | ND | 221 |
| | | pg/MM | IU/day | pg/MM | | pg/MM | IU/day | | pg/MM |
| | | WBC | | WBC | | WBC | | | WBC |
| | DHA | 1.2 | 5 | 1.9 | 0.14 | 9 | 50.71 | ND | 9.4 |
| | | | mg/day | | | | mg/day | | |
| Serum | Folate | 0.9 | 50 | 3.3 | 0.048 | 12.5 | 191.66 | 1,000 | 14.5 |
| | | ng/mL | mcg/day | ng/mL | | ng/mL | mcg/day | mcg/day | ng/mL |
| | Manganese | 0.4 | 0.95 | 0.62 | 0.23157895 | 2.0 | 5.95 | 11 | 2.1 |
| | | ng/mL | mg/day | ng/mL | | ng/mL | mg/day | mg/day | ng/mL |
| | L-Arginine | 58 | 10 | 67 | 0.9 | 225 | 175.55 | 20000 | 232 |
| | | nmol/mL | mg/kg/day | nmol/mL | | nmol/mL | mg/kg/day | mg/day | nmol/mL |
| | Vitamin B1 | 1.1 | 0.2 | 12.3 | 56 | 85 | 1.29 | ND | 92.5 |
| | | nmol/L | mg/day | nmol/L | | nmol/L | mg/day | | nmol/L |
| | Vitamin B12 | 94 | 0.4 | 147 | 132.5 | 1100 | 7.19 | ND | 1122 |
| | | ng/L | mcg/day | ng/L | | ng/L | mcg/day | | ng/L |
| | Calcium | 5.4 | 1 | 5.9 | 0.5 | 10.32 | 8.84 | 2,500 | 10.42 |
| | | mg/dL | mg/day | mg/dL | | mg/dL | mg/day | mg/day | mg/dL |
| | Vitamin B5 | 14.4 | 0.5 | 23.5 | 18.2 | 290 | 14.64 | ND | 311 |
| | | mcg/L | mg/day | mcg/L | | mcg/L | mg/day | | mcg/L |
| | Coenzyme 10 | 0.1 | 5 | 1.2 | 0.22 | 1.7 | 2.27 | 1,200 | 1.88 |
| | | mcg/mL | mg/day | mcg/mL | | mcg/mL | mg/day | mg/day | mcg/mL |
| | Selenium | 105 | 7.5 | 122 | 2.26666667 | 255 | 58.67 | 400 | 267 |
| | | ng/mL | mcg/day | ng/mL | | ng/mL | mcg/day | mcg/day | ng/mL |
| | Citrulline | 9.1 | 10 | 16.7 | 0.76 | 41 | 31.97 | ND | 43.4 |
| | | nmol/mL | mg/kg/day | nmol/mL | | nmol/mL | mg/kg/day | | nmol/mL |
| | Vitamin A | 22 | 1,000 | 47 | 0.025 | 125 | 3120 | 10,000 | 129 |
| | | mcg/dL | IU/day | mcg/dL | | mcg/dL | IU/day | IU/day | mcg/dL |
| | Vitamin K1 | 0.08 | 20 | 1.7 | 0.081 | 11.5 | 120.98 | ND | 12.4 |
| | | ng/ml | mcg/day | ng/ml | | ng/mL | mcg/day | | ng/mL |
| | Vitamin E | 3.2 | 3 | 17.8 | 4.86666667 | 34.5 | 3.43 | 1,100 | 35.4 |
| | | mg/L | IU/day | mg/L | | mg/L | IU/day | IU/day | mg/L |
| | Isoleucine | 14.2 | 3.5 | 44 | 8.51428571 | 140 | 11.27 | ND | 149 |
| | | nmol/mL | mg/kg/day | nmol/mL | | nmol/mL | mg/kg/day | | nmol/mL |
| | Myo- | 7.4 | 10 | 12.9 | 0.55 | 45 | 58.36 | ND | 47.5 |
| | Inositol | nmol/mL | mg/day | nmol/mL | | nmol/mL | mg/day | | nmol/mL |
| Genetics | Vitamin A | Homozygous | | | | | | | |
| | | mutant | | | | | | | |
| | Folate | Heterozygous | | | | | | | |
| | | mutant | | | | | | | |
| | Selenium | Heterozygous | | | | | | | |
| | | mutant | | | | | | | |
| | Vitamin C | Heterozygous | | | | | | | |
| | | mutant | | | | | | | |
| | Vitamin D3 | Homozygous | | | | | | | |
| | | mutant | | | | | | | |
| Questionnaire | Zinc | 0.75 | 1.5 | 1.1 | 0.233333333 | 1.45 | 1.5 | 40 | 1.47 |
| | | mcg/mL | mg/day | mcg/mL | | mcg/mL | mg/day | mg/day | mcg/mL |
| | Iodine | RDA: 150 | | | | | | | |
| | | mcg/day | | | | | | | |

TABLE 20

| | | Actual | | | | Multipli- | Customized | | | |
|---|---|---|---|---|---|---|---|---|---|---|
| Level | | Nutrient | | Target | | cation | Blend | | Follow-up | |
| (35-year-old | | Value | Nutrient | Nutrient | | Factor | (Dosage × | | Nutrient | Nutrient |
| Male) | Nutrient | (ANV) | Zone | Zone | Dosage | (MF) | MF) | UTL | Level | Zone |
| Cells | Vitamin A | 0.4 | Zone 1 | Zone 4 | 1,000 | 3 | 3000 | 10,000 | 14.4 | Zone 4 |
| | | pg/MM | | | IU/day | | IU/day | IU/day | pg/MM | |
| | | WBC | | | | | | | WBC | |
| | Vitamin B1 | 0.08 | Zone 1 | Zone 4 | 0.2 | 3.5 | 0.7 | ND | 3.23 | Zone 3 |
| | | pg/MM | | | mg/day | | mg/day | | pg/MM | |
| | | WBC | | | | | | | WBC | |
| | DPA | 0.46 | Zone 2 | Zone 5 | 0.5 | 5.5 | 2.75 | ND | 1.2 | Zone 3 |
| | | | | | mg/day | | mg/day | | | |
| | Iron | 62 | Zone 1 | Zone 4 | 2 | 4.4 | 8.8 | 45 | 114.1 | Zone 4 |
| | | mg/dL | | | mg/day | | mg/day | mg/day | mg/dL | |
| | LA | 3.5 | Zone 2 | Zone 5 | 5 | 5.5 | 27.5 | ND | 6.1 | Zone 3 |
| | (Linoleic | | | | mg/day | | mg/day | | | |
| | Acid) | | | | | | | | | |
| Serum | Vitamin A | 35 | Zone 1 | Zone 4 | 1,000 | 3 | 3000 | 10,000 | 128 | Zone 4 |
| | | mcg/dL | | | IU/day | | IU/day | IU/day | mcg/dL | |
| | Vitamin B1 | 29.6 | Zone 1 | Zone 4 | 0.2 | 3.5 | 0.7 | ND | 62 | Zone 3 |
| | | nmol/L | | | mg/day | | mg/day | | nmol/L | |
| | Zinc | 0.5 | Zone 1 | Zone 4 | 1.5 | 3.5 | 5.25 | 40 | 1.55 | Zone 4 |
| | | mcg/mL | | | mg/day | | mg/day | mg/day | mcg/mL | |
| | Selenium | 190 | Zone 1 | Zone 4 | 7.5 | 8 | 60 | 400 | 267 | Zone 4 |
| | | ng/mL | | | mcg/day | | mcg/day | mcg/day | ng/mL | |
| | Coenzyme | 0.1 | Zone 1 | Zone 4 | 5 | 12.5 | 62.5 | 1,200 | 1.99 | Zone 4 |
| | Q10 | mcg/mL | | | mg/day | | mg/day | mg/day | mcg/mL | |
| | Valine | 26 | Zone 1 | Zone 4 | 4 | 4.5 | 18 | ND | 332 | Zone 4 |
| | | nmol/mL | | | mg/kg/day | | mg/kg/day | | nmol/mL | |
| | Isoleucine | 26.2 | Zone 2 | Zone 4 | 3.5 | 4.5 | 15.75 | ND | 144 | Zone 4 |
| | | nmol/mL | | | mg/kg/day | | mg/kg/day | | nmol/mL | |
| | Leucine | 85 | Zone 1 | Zone 4 | 3.5 | 4.5 | 15.75 | 35 | 225 | Zone 4 |
| | | nmol/m | | | mg/kg/day | | mg/kg/day | g/d | nmol/m | |
| | Vitamin K1 | 0.07 | Zone 1 | Zone 4 | 20 | 3.5 | 70 | ND | 12.3 | Zone 4 |
| | | ng/ml | | | mcg/day | | mcg/day | | ng/mL | |
| | Iron | 22 | Zone 1 | Zone 4 | 2 | 4.4 | 8.8 | 45 | 141 | Zone 4 |
| | | mcg/dL | | | mg/day | | mg/day | mg/day | mcg/dL | |
| | Chromium | 0.133 | Zone 2 | Zone 4 | 10 | 3.5 | 35 | ND | 0.277 | Zone 4 |
| | | ng/mL | | | mcg/day | | mcg/day | | ng/mL | |
| | Vitamin | 130 | Zone 2 | Zone 4 | 0.25 | 40 | 10 | 100 | 401 | Zone 4 |
| | B6 | ng/mL | | | mg/day | | mg/day | mg/day | ng/mL | |
| | Vitamin B2 | 5.5 | Zone 1 | Zone 4 | 0.25 | 3.5 | 0.875 | ND | 231 | Zone 4 |
| | | mcg/L | | | mg/day | | mg/day | | mcg/L | |
| | Serine | 62 | Zone 2 | Zone 4 | 5 | 4.5 | 22.5 | 12000 | 131 | Zone 4 |
| | | nmol/mL | | | mg/kg/day | | mg/kg/day | mg/day | nmol/mL | |
| | L-Glutamine | 290 | Zone 1 | Zone 4 | 5 | 4.5 | 22.5 | 14000 | 677 | Zone 4 |
| | | nmol/mL | | | mg/day | | mg/day | mg/day | nmol/mL | |
| Genetics | Vitamin A | Homozygous | | | | | | | | |
| | | mutant | | | | | | | | |
| | Vitamin B1 | Homozygous | | | | | | | | |
| | | mutant | | | | | | | | |
| | Iron | Homozygous | | | | | | | | |
| | | mutant | | | | | | | | |
| | Selenium | Heterozygous | | | | | | | | |
| | | mutant | | | | | | | | |
| | Vitamin A | Homozygous | | | | | | | | |
| | | mutant | | | | | | | | |
| Questionnaire | Carnitine | 24 | Zone 2 | Zone 4 | 5 | 4.5 | 22.5 | 2000 | 43.2 | Zone 4 |
| | | nmol/mL | | | mg/day | | mg/day | mg/day | nmol/mL | |

TABLE 21

| | | Actual | | | | Multipli- | Customized | | Follow- | |
|---|---|---|---|---|---|---|---|---|---|---|
| Level | | Nutrient | | Target | | cation | Blend | | up | |
| (70-year-old | | Value | Nutrient | Nutrient | | Factor | (Dosage × | | Nutrient | Nutrient |
| Female) | Nutrient | (ANV) | Zone | Zone | Dosage | (MF) | MF) | UTL | Level | Zone |
| Cells | Iron | 76.5 | Zone 1 | Zone 4 | 2 | 4.4 | 8.8 | 45 | 76.5 | Zone 1 |
| | | mg/dL | | | mg/day | | mg/day | mg/day | mg/dL | |
| | Calcium | 4.2 | Zone 1 | Zone 4 | 1 | 1.8 | 1.8 | 2,500 | 4.2 | Zone 1 |
| | | ng/MM | | | | | mg/day | mg/day | ng/MM | |
| | | WBC | | | mg/day | | | | WBC | |
| | Vitamin B1 | 0.05 | Zone 1 | Zone 4 | 0.2 | 3.5 | 0.7 | ND | 0.05 | Zone 1 |
| | | pg/MM | | | mg/day | | mg/day | | pg/MM | |
| | | WBC | | | | | | | WBC | |
| | EPA | 0.05 | Zone 1 | Zone 5 | 5 | 8 | 40 | ND | 0.05 | Zone 1 |
| | | | | | mg/day | | mg/day | | | |
| | DHA | 0.9 | Zone 1 | Zone 5 | 5 | 8 | 40 | ND | 0.9 | Zone 1 |
| | | | | | mg/day | | mg/day | | | |
| Serum | Vitamin B5 | 5.4 | Zone 1 | Zone 4 | 0.5 | 3.5 | 1.75 | ND | 14.4 | Zone 1 |
| | | mcg/L | | | mg/day | | mg/day | | mcg/L | |
| | Potassium | 2.8 | Zone 1 | Zone 4 | 10 | 1 | 10 | ND | 3.4 | Zone 1 |
| | | mmol/L | | | mg/day | | mg/day | | mmol/L | |
| | L-Serine | 44 | Zone 1 | Zone 4 | 5 | 4.5 | 22.5 | 12000 | 51 | Zone 1 |
| | | nmol/mL | | | mg/kg/day | | mg/kg/day | mg/day | nmol/mL | |
| | Calcium | 3.1 | Zone 1 | Zone 4 | 1 | 1.8 | 1.8 | 2,500 | 6.2 | Zone 1 |
| | | mg/dL | | | mg/day | | mg/day | mg/day | mg/dL | |
| | Iron | 14 | Zone 1 | Zone 4 | 2 | 4.4 | 8.8 | 45 | 19 | Zone 1 |
| | | mcg/dL | | | mg/day | | mg/day | mg/day | mcg/dL | |
| | Vitamin D3 | 0.1 | Zone 1 | Zone 5 | 100 | 25 | 2500 | ND | 0.72 | Zone 1 |
| | | ng/mL | | | IU/day | | IU/day | | ng/mL | |
| | Folate | 0.8 | Zone 1 | Zone 4 | 50 | 2.4 | 120 | 1,000 | 1.2 | Zone 1 |
| | | ng/mL | | | mcg/day | | mcg/day | mcg/day | ng/mL | |
| | Vitamin B1 | 0.9 | Zone 1 | Zone 4 | 0.2 | 3.5 | 0.7 | ND | 2.3 | Zone 2 |
| | | nmol/L | | | mg/day | | mg/day | | nmol/L | |
| | Copper | 0.1 | Zone 1 | Zone 4 | 0.08 | 40 | 3.2 | 10 | 0.5 | Zone 1 |
| | | mcg/mL | | | mg/day | | mg/day | mg/day | mcg/mL | |
| | L-Glutamine | 302 | Zone 2 | Zone 4 | 5 | 4.5 | 22.5 | 14000 | 371 | Zone 2 |
| | | nmol/mL | | | mg/day | | mg/day | mg/day | nmol/mL | |
| | Vitamin E | 4.2 | Zone 1 | Zone 4 | 3 | 40 | 120 | 1,100 | 5.4 | Zone 1 |
| | | mg/L | | | IU/day | | IU/day | IU/day | mg/L | |
| | Vitamin B6 | 0.5 | Zone 1 | Zone 4 | 0.25 | 40 | 10 | 100 | 0.9 | Zone 2 |
| | | ng/mL | | | mg/day | | mg/day | mg/day | ng/mL | |
| | Vitamin C | 0.1 | Zone 1 | Zone 4 | 15 | 25 | 375 | 2000 | 0.23 | Zone 1 |
| | | mg/dL | | | mg/day | | mg/day | mg/day | mg/dL | |
| Genetics | Iron | Heterozygous | | | | | | | | |
| | | mutant | | | | | | | | |
| | Potassium | Homozygous | | | | | | | | |
| | | mutant | | | | | | | | |
| | Calcium | Homozygous | | | | | | | | |
| | | mutant | | | | | | | | |
| Questionnaire | Arginine | 84 | Zone 2 | Zone 4 | 10 | 4.5 | 45 | 20000 | 89.5 | Zone 2 |
| | | nmol/mL | | | mg/kg/day | | mg/kg/day | mg/day | nmol/mL | |
| | Citrulline | 19.2 | Zone 2 | Zone 4 | 10 | 4.5 | 45 | ND | 24.9 | Zone 2 |
| | | nmol/mL | | | mg/kg/day | | mg/kg/day | | nmol/mL | |

1. A method for providing a nutritional supplement for a subject, the method comprising:
obtaining or having obtained levels of a plurality of extracellular nutrients measured from serum obtained from the subject;
obtaining or having obtained levels of a plurality of intracellular nutrients measured from intracellular white blood cells (WBCs) and/or red blood cells (RBCs) obtained from the subject; and
determining a personalized dosage of the nutritional supplement for the subject using both of the levels of the plurality of extracellular nutrients and the levels of the plurality of intracellular nutrients.

2. The method of claim 1, wherein:
the extracellular nutrients comprise one or more of vitamins, minerals, amino acids, and fatty acids,
the intracellular nutrients comprise one or more of vitamins, minerals, amino acids, and fatty acids, or
both the extracellular nutrients and the intracellular nutrients each comprise one or more of vitamins, minerals, amino acids, and fatty acids.

3. The method of claim 1, wherein determining the personalized dosage of the nutritional supplement comprises:
for one of an extracellular nutrient or an intracellular nutrient, determining a ratio of a target nutrient value and the obtained level of the extracellular nutrient or intracellular nutrient, or
for an extracellular nutrient, determining a ratio of a target extracellular nutrient value and the obtained level of the extracellular nutrient, and for an intracellular nutrient, determining a ratio of a target intracellular nutrient value and the obtained level of the intracellular nutrient.

4. The method of claim 1, further comprising:
obtaining or having obtained genetic information of the subject, the genetic information relating to inhibition or absorption of nutrients,
wherein the genetic information of the subject comprises genetic statuses of one or more genomic locations of a plurality of genes, and the genetic statuses comprise presence or absence of a polymorphism.

5. The method of claim 4, further comprising:
determining a polygenic risk factor according to the genetic statuses of the one or more locations of the plurality of genes, wherein the personalized dosage of the nutritional supplement is determined according to the polygenic risk factor, and
wherein determining the polygenic risk factor comprises:
for each of the one or more locations of a gene, assigning a genotype score according to presence or absence of a polymorphism at the location of the gene; and
combining the genotype scores across the locations of the plurality of genes.

6. The method of claim 1, further comprising obtaining or having obtained body metrics of the subject, wherein the body metrics of the subject comprise one or more of height and weight, and the personalized dosage of the nutritional supplement is determined according to the body metrics of the subject.

7. The method of claim 1, further comprising obtaining or having obtained an absorption factor determined for the subject, the absorption factor reflecting subject's utilization of a plurality of nutrients, wherein the personalized dosage of the nutritional supplement for the subject is determined according to the absorption factor, and the absorption factor for the subject is determined by comparing pre-supplementation blood nutrient values to post-supplementation blood nutrient values.

8. The method of claim 1, further comprising obtaining a recommended dietary allowance (RDA) value for the subject, wherein the RDA value is determined according to the subject's age, gender, or pregnancy or lactation status, and the personalized dosage of the nutritional supplement for the subject is determined according to the RDA value.

9. The method of claim 1, further comprising obtaining or having obtained a measure of the subject's gut microbiome, wherein determining the personalized dosage further comprises determining the amount of probiotics or prebiotics of the nutritional supplement for the subject according to the subject's gut microbiome.

10. The method of claim 1, further comprising:
obtaining or having obtained one or more of:
biomarkers of the subject indicative of cardiovascular health;
biomarkers of the subject indicative of neurological health;
biomarkers of the subject indicative of thyroid health;
biomarkers of the subject indicative of kidney health; and
biomarkers of the subject indicative of liver health.

11. The method of claim 10, wherein determining the personalized dosage further comprises determining the personalized dosage of the nutritional supplement for the subject according to the one or more of:
levels of nutrients and/or supporting supplements supportive of cardiovascular health;
levels of nutrients and/or supporting supplements supportive of neurological health;
levels of nutrients and/or supporting supplements supportive of thyroid health;
levels of nutrients and/or supporting supplements supportive of kidney health; and
levels of nutrients and/or supporting supplements supportive of liver health.

12. The method of claim 1, further comprising administering or having administered the personalized dosage of the nutritional supplement to the subject.

13. The method of claim 12, further comprising:
periodically obtaining or having obtained post-supplementation blood nutrient values after administration of the personalized dosage of the nutritional supplement to the subject; and
determining whether to modify the personalized dosage of the nutritional supplement based on the post-supplementation blood nutrient values.

14. The method of claim 12, wherein, before obtaining or having obtained levels of a plurality of extracellular nutrients measured from serum and a plurality of intracellular nutrients measured from RBC and/or WBC obtained from the subject, the method further comprises administering an Absorption Testing Blend (ATB) to the subject for a first predefined period, wherein the ATB comprises a multi-nutrient blend comprised of vitamins, minerals, amino acids and fatty acids.

15. The method of claim 14, further comprising obtaining or having obtained pre-supplementation blood nutrient values and post-supplementation blood nutrient values, wherein the pre-supplementation blood nutrient values are determined before administering the ATB to subject and the post-supplementation blood nutrient values are determined after administering the ATB for the first predefined period.

16. The method of claim 15, wherein determining a personalized dosage of the nutritional supplement comprises:
determining a vibrant absorption factor (VAF) based on a difference between pre-supplementation nutrient values and post-supplementation nutrient values, divided by an ATB dosage;
determining a target nutrient value (TNV) for each nutrient in both cellular and serum contexts; and
determining a personalized dosage for each nutrient based on a difference between the TNV and the post-supplementation nutrient values, divided by the VAF.

17. The method of claim 12, further comprising administering the personalized dosage of the nutritional supplement to the subject with one or more predefined supplements.

18. The method of claim 1, further comprising dividing a nutrient reference range for individuals into a number of zones with different nutrient wellness levels.

19. The method of claim 18, wherein determining the personalized dosage of the nutritional supplement for the subject comprises:
determining a current zone of a nutrient for the subject using both of the levels of the plurality of extracellular nutrients and the levels of the plurality of intracellular nutrients;
determining a target nutrient zone for the nutrient;
determining a multiplication factor for the nutrient based on the current zone and the target nutrient zone for the nutrient; and
determine the personalized dosage of the nutritional supplement for the subject based on the multiplication factor for the nutrient.

20. The method of claim 19, wherein determining the personalized dosage of the nutritional supplement for the subject based on the multiplication factor comprises multiplying a starting dosage for the nutrient with the multiplication factor.
